(19) United States
(12) Patent Application Publication
(10) Pub. No.: US 2022/0387571 A1
(43) Pub. Date: Dec. 8, 2022

(54) THERAPY FOR HEMATOPOIETIC CELL MALIGNANCIES USING GENETICALLY ENGINEERED T CELLS TARGETING CD70

(71) Applicant: CRISPR THERAPEUTICS AG, Zug (CH)

(72) Inventors: Jonathan Alexander TERRETT, Cambridge, MA (US); Mary-Lee DEQUÉANT, Cambridge, MA (US); Matthias WILL, Cambridge, MA (US)

(73) Assignee: CRISPR THERAPEUTICS AG, Zug (CN)

(21) Appl. No.: 17/776,846

(22) PCT Filed: Nov. 13, 2020
(86) PCT No.: PCT/IB2020/060718

§ 371 Date: May 13, 2022

Related U.S. Application Data
(60) Provisional application No. 63/034,510, filed on Jun. 4, 2020.
(60) Provisional application No. 62/934,945, filed on Nov. 13, 2019.

(51) Int. Cl. A61K 39/00; A61K 31/7076; A61K 31/675

(52) U.S. Cl. CPC A61K 39/001138; A61K 31/7076; A61K 31/675; A61K 2039/5156; A61K 2039/5158; A61K 2039/804; A61P 35/00

(57) ABSTRACT

Aspects of the present disclosure relate to compositions comprising a population of genetically engineered T cells that expresses a chimeric antigen receptor (CAR) that binds CD70, and methods of using such for the treatment of T cell and B cell malignancies.

### CROSS-REFERENCE TO RELATED APPLICATIONS

This application claims the benefit of priority to U.S. Provisional Patent Application No. 62/934,945, filed Nov. 13, 2019, and U.S. Provisional Patent Application No. 63/034,510, filed Jun. 4, 2020. Each of the prior applications is hereby incorporated by reference in its entirety.

### BACKGROUND

Chimeric antigen receptor (CAR) T-cell therapy uses genetically-modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

### SUMMARY

The present disclosure is based, at least in part, on the surprising discovery that anti-CD70 CAR+ T cells, such as CTX130 cells disclosed herein, provided long-term tumor elimination in a subcutaneous T cell lymphoma xenograft model. For example, anti-CD70 CAR+ T cells described herein (e.g., CTX130 cells) provided complete tumor elimination for at least 90 days following administration. Significant reductions in tumor burden were also observed in an additional subcutaneous T cell lymphoma xenograft model. Further, CTX130 cell distribution, expansion, and persistence were observed in human subjects receiving the CAR-T cells. Superior treatment efficacy was also observed in human lymphoma patients who received the CTX130 cell treatment.

Accordingly, the present disclosure provides, in some aspects, a method for treating a hematopoietic cell malignancy (e.g., T cell or B cell malignancy, or myeloid cell malignancy) the method comprising: (i) subjecting a human patient (e.g., a human adult patient) having a hematopoietic cell malignancy to a first lymphodepletion treatment; and (ii) administering to the human patient a first dose of a population of genetically engineered T cells after step (i). The population of genetically engineered T cells comprises T cells expressing a chimeric antigen receptor (CAR) that binds CD70, a disrupted TRAC gene, a disrupted β2M gene, and a disrupted CD70 gene, and wherein a nucleotide sequence encoding the CAR is inserted into the disrupted TRAC gene. In some instances, the population of genetically engineered T cells are CTX130 cells as disclosed herein. In some embodiments, step (i) can be performed about 2-7 days prior to step (ii).

In some embodiments, the first lymphodepletion treatment in step (i) comprises co-administering to the human patient fludarabine at 30 mg/m² and cyclophosphamide at 500 mg/m² intravenously per day for three days. Alternatively or in addition, step (ii) is performed by administering the population of genetically engineered T cells to the human patient intravenously at the first dose, which may be about 1×10⁷ CAR⁺ cells to about 1×10⁹ CAR⁺ cells. In some instances, the first dose may range from about 3×10⁷ to about 9×10⁸ CAR⁺ cells.

In some embodiments, prior to step (i), the human patient does not show one or more of the following features: (a) change in performance status to ECOG>1, (b) significant worsening of clinical status, (c) requirement for supplemental oxygen to maintain a saturation level of greater than 92%, (d) uncontrolled cardiac arrhythmia, (e) hypotension requiring vasopressor support, (f) active infection, and (g) any acute neurological toxicity (e.g., ≥2 acute neurological toxicity).

In some embodiments, prior to step (ii) and after step (i), the human patient does not show one or more of the following features: (a) change in performance status to Eastern Cooperative Oncology Group (ECOG)>1; (b) active uncontrolled infection, (c) significant worsening of clinical status, and (d) any acute neurological toxicity (e.g., ≥2 acute neurological toxicity).

Any of the methods disclosed herein may further comprise monitoring the human patient for development of acute toxicity after step (ii). Exemplary acute toxicities may comprise cytokine release syndrome (CRS), neurotoxicity, tumor lysis syndrome, GvHD, on target off-tumor toxicity, uncontrolled T cell proliferation, or a combination thereof.

In some instances, the method disclosed herein may further comprise subjecting the human patient to a second lymphodepletion treatment, and administering to the human patient a second dose of the population of genetically engineered T cells after step (ii). In some instances, the second dose is administered to the human patient about 8 weeks to about 2 years after the first dose. In some examples, the human patient eligible for the second dose of the genetically engineered T cells does not show one or more of the following after step (ii): (a) dose-limiting toxicity (DLT), (b) grade>1 GvHD, (c) grade 4 CRS that does not resolve to grade 2 within 72 hours, (d) grade≥3 neurotoxicity; (e) active infection, (f) hemodynamically unstable, and (g) organ dysfunction. In some examples, the second lymphodepletion treatment in step (iv) comprises co-administering to the human patient fludarabine at 30 mg/m² and cyclophosphamide at 500 mg/m² intravenously per day for 1-3 days. In some examples, the second dose of the genetically engineered T cells can be administered to the human patient 2-7 days after the second lymphodepletion treatment. In some examples, the second dose of the population of genetically engineered T cells can be administered to the human patient intravenously at about 1×10⁷ CAR⁺ cells to about CAR⁺ 1×10⁹ cells. For example, the second dose may range from about 3×10⁷ to about 9×10⁸ CAR+ cells.

In some instances, the method may further comprise subjecting the human patient to a third lymphodepletion treatment, and administering to the human patient a third dose of the population of genetically engineered T cells. In some examples, the third dose can be administered to the human patient about 8 weeks to about 2 years after the second dose. The human patient may receive the first, second, and third doses of the population of genetically engineered T cells in three months, and may not show one or more of the following after the second dose of the genetically engineered T cells: (a) dose-limiting toxicity (DLT), (b) grade 4 CRS that does not resolve to grade 2 within 72 hours, (c) grade≥1 GvHD, (d) grade≥3 neurotoxicity, (e) active infection, (f) hemodynamically unstable, and (g) organ dysfunction. In some examples, the third lymphodepletion treatment may comprise co-administering to the human patient fludarabine at 30 mg/m² and cyclophosphamide at 500 mg/m² intravenously per day for 1-3 days. In some instances, the third dose of the genetically engineered T cells may be administered to the human patient 2-7 days after the third lymphodepletion treatment. In some examples, the third dose of the population of genetically engineered T cells can be administered to the human patient intravenously at about 1×10⁷ CAR⁺ cells to about CAR⁺ 1×10⁹ cells. For example, the third dose may range from about 3×10⁷ to about 9×10⁸ CAR⁺ cells.

Any of the human patient receiving the second and/or third doses of the genetically engineered T cells may show stable disease or disease progress.

In some examples, the first dose, the second dose, and/or the third dose of the population of genetically engineered T cells is 1×10⁷ CAR⁺ cells. In some examples, the first dose, the second dose, and/or the third dose of the population of genetically engineered T cells is about 3×10⁷ CAR⁺ cells. In some examples, the first dose, the second dose, and/or the third dose of the population of genetically engineered T cells is about 1×10⁸ CAR⁺ cells. In some examples, the first dose, the second dose, and/or the third dose of the population of genetically engineered T cells is about 1.5×10⁸ CAR⁺ cells. In some examples, the first dose, the second dose, and/or the third dose of the population of genetically engineered T cells is about 3×10⁸ CAR⁺ cells. In some examples, the first dose, the second dose, and/or the third dose of the population of genetically engineered T cells is about 4.5×10⁸ CAR⁺ cells. In some examples, the first dose, the second dose, and/or the third dose of the population of genetically engineered T cells is about 6×10⁸ CAR⁺ cells. In some examples, the first dose, the second dose, and/or the third dose of the population of genetically engineered T cells is about 7.5×10⁸ CAR⁺ cells. In some examples, the first dose, the second dose, and/or the third dose of the population of genetically engineered T cells is about 9×10⁸ CAR⁺ cells. In some examples, the first dose, the second dose, and/or the third dose of the population of genetically engineered T cells is about 1×10⁹ CAR⁺ cells.

In some instances, the first dose of the population of genetically engineered T cells is the same as the second and/or third dose of the population of genetically engineered T cells. In other instances, the first dose of the population of genetically engineered T cells is lower than the second and/or third dose of the population of genetically engineered T cells.

In any of the methods disclosed herein, the human patient may have undergone a prior anti-cancer therapy. Alternatively or in addition, the human patient may have relapsed or refractory hematopoietic cell malignancies.

In some embodiments, the human patient has a T cell malignancy, e.g., a relapsed or refractory T cell malignancy. In some examples, the human patient has cutaneous T-cell lymphoma (CTCL). Such a human patient may have mycosis fungoides (MF), for example, stage IIb or higher, including transformed large cell lymphoma. Alternatively, the human patient may have Sezary Syndrome (SS). In other examples, the human patient has peripheral T-cell lymphoma (PTCL). Examples include, but are not limited to, angioimmunoblastic T cell lymphoma (AITL), anaplastic large cell lymphoma (ALCL), which may be Alk positive or Alk negative, adult T cell leukemia or lymphoma (ATLL), which may exclude the smoldering subtype (non-smoldering ATLL); and peripheral T-cell lymphoma not otherwise (PTCL-NOS).

In some examples, the human patient has PTCL, ATLL, or AITL and has failed a first line systemic therapy. In some examples, the human patient has ALCL and has failed a combined therapy comprising breutuximab vedotin. In some examples, the human patient has ALK⁺ ALCL and has failed two prior lines of therapy, one of which comprises brentuximab vedotin. In other examples, the human patient has ALK⁻ ALCL and has failed one prior line of therapy. In yet other examples, the human patient has MF or SS and has failed a prior systemic therapy or a prior mogamulizumab therapy.

In some embodiments, the human patient may have a B cell malignancy, for example, a relapsed or refractory B cell malignancy. In some examples, the human patient has diffused large B cell lymphoma (DLBCL). Such a human patient may have failed a prior anti-CD19 CAR-T cell therapy. In other examples, the human patient has mantle cell lymphoma (MCL).

In yet other embodiments, the human patient may have a myeloid cell malignancy, for example, a relapsed or refractory myeloid cell malignancy. In some examples, the human patient has acute myeloid leukemia (AML).

Any of the human patients to be treated by the method disclosed herein may be free of mogamulizumab treatment at least three months prior to the first dose of the population of genetically modified T cells.

In any of the methods disclosed herein, the human patient may have CD70+ tumor cells. For example, the human patient may have at least 10% CD70⁺ tumor cells in a biological sample obtained from the human patient. In some instances, the biological sample is a tumor tissue sample and the level of CD70+ tumor cells is measured by immunohistochemistry (IHC). In other instances, the biological sample is a blood sample or a bone marrow sample and the level of CD70+ tumor cells is determined by flow cytometry. Any of the methods disclosed herein may further comprise, prior to step (i), identifying a human patient having CD70+ tumor cells involved in a T cell or B cell malignancy.

Alternatively or in addition, the human patient to be treated by the method disclosed herein may be subject to an anti-cytokine therapy. In some examples, the human patient has one or more of the following features: (a) adequate organ function, (b) free of a prior stem cell transplantation (SCT), (c) free of a prior anti-CD70 agent or adoptive T cell or NK cell therapy, (d) free of known contraindication to a lymphodepletion therapy, (e) free of T cell or B cell lymphomas with a present or a past malignant effusion that is or was symptomatic, (f) free of hemophagocytic lymphohistiocytosis (HLH), (g) free of central nervous system malignancy or disorders, (h) free of unstable angina, arrhythmia, and/or myocardial infarction, (i) free of diabetes mellitus, j) free of uncontrolled infections, (k) free of immunodeficiency disorders or autoimmune disorders that require immunosuppressive therapy, and (l) free of solid organ transplantation.

In any of the methods disclosed herein, the human patient can be monitored for at least 28 days for development of toxicity after each administration of the population of genetically engineered T cells. If development of toxicity is observed, the human patient can be subject to toxicity management.

The genetically engineered T cells may express a CAR binding to CD70. The CAR may comprises an extracellular domain, a CD8 transmembrane domain, a 4-1BB co-stimulatory domain, and a CD3ζ cytoplasmic signaling domain. In some instances, the extracellular domain is a single-chain antibody fragment (scFv) that binds CD70. In some examples, the scFv comprises a heavy chain variable domain (V_{H}) comprising SEQ ID NO: 49, and a light chain variable domain (V_{L}) comprising SEQ ID NO: 50. In some examples, the scFv comprises SEQ ID NO: 48. In some specific examples, the CAR comprises SEQ ID NO: 46.

In some embodiments, the genetically engineered T cells have a disrupted TRAC gene, which may be produced by a CRISPR/Cas9 gene editing system. In some examples, the CRISPR/Cas9 gene editing system may comprise a guide RNA comprising a spacer sequence of SEQ ID NO: 8 or 9. In some examples, the disrupted TRAC gene has a deletion of the region targeted the spacer sequence of SEQ ID NO: 8, or a portion thereof.

In some embodiments, the genetically engineered T cells have a disrupted β2M gene, which may be produced by a CRISPR/Cas9 gene editing system. In some examples, the CRISPR/Cas9 gene editing system may comprise a guide RNA comprising a spacer sequence of SEQ ID NO: 12 or 13.

In some embodiments, the genetically engineered T cells have a disrupted CD70 gene, which may be produced by a CRISPR/Cas9 gene editing system. In some examples, the CRISPR/Cas9 gene editing system may comprise a guide RNA comprising a spacer sequence of SEQ ID NO: 4 or 5.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 includes graphs showing efficient multiple gene editing in TRAC⁻/β2M⁻/CD70⁻/anti-CD70 CAR⁺ (i.e., 3×KO (CD70), CD70 CAR⁺) T cells.

FIG. 2 includes a graph showing that normal proportions of CD4+ and CD8+ T cells are maintained among the TRAC⁻/β2M⁻/CD70⁻/anti-CD70 CAR⁺ T cell population.

FIG. 3 includes a graph showing robust cell expansion in TRAC⁻/β2M⁻/CD70⁻/anti-CD70 CAR⁺ T cells. The total number of viable cells was quantified in 3×KO (TRAC−/β2M−/CD70−) and 2×KO (TRAC−/β2M−) anti-CD70 CAR T cells. 3×KO cells were generated with either CD70 sgRNA T7 or T8.

FIGS. 4A-4K includes graphs showing relative CD70 expression in various cancer cell lines. FIG. 4A: graph showing relative CD70 expression in nine different cancer cell lines. FIG. 4B: a graph showing cell kill activity using the triple knockout TRAC⁻/β2M⁻/CD70⁻/anti-CD70 CAR⁺ T cells (3KO (CD70), CD70 CAR+) against CD70-deficient chronic myelogenous leukemia (K562) cells at various effector:target ratios. FIG. 4C: a graph showing cell kill activity of the same triple knockout TRAC⁻/β2M⁻/CD70⁻/anti-CD70 CAR⁺ T cells (3KO (CD70), CD70 CAR+) against CD70-expressing multiple myeloma (MM.1S) cells at various effector:target ratios. FIG. 4D: a graph showing cell kill activity of the same triple knockout TRAC⁻/β2M⁻/CD70⁻/anti-CD70 CAR⁺ T cells (3KO (CD70), CD70 CAR+) against CD70-expressing T cell lymphoma (HuT78) cells at various effector:target ratios. FIG. 4E: a graph showing cell kill activity of the same triple knockout TRAC⁻/β2M⁻/CD70⁻/anti-CD70 CAR⁺ T cells (3KO (CD70), CD70 CAR+) against high CD70-expressing T cell lymphoma cells (MJ), lower CD70-expressing T cell lymphoma cells (HuT78), and non-CD70 expressing negative control cells (K562) at various effector:target ratios. FIGS. 4F-4K: graphs showing cell kill activity of TRAC⁻/β2M⁻/CD70⁻/anti-CD70 CAR⁺ T cells (e.g.: CTX130) in various types of acute myeloid leukemia cell lines, including MV411 (FIG. 4F), EOL-1 (FIG. 4G), HL60 (FIG. 4H), Kasumi-1 (FIG. 4H), KG1 (FIG. 4J), and THP-1 cells (FIG. 4K).

FIGS. 5A-5B include graphs showing anti-tumor activity of anti-CD70 CAR+ T cells, e.g., CTX130 cells. FIG. 5A: graph showing tumor volume reduction in a human T-cell lymphoma xenograft model (e.g., HuT78 tumor cells) exposed to TRAC−/B2M−/CD70− anti-CD70 CAR+ T cells, e.g., CTX130 cells. FIG. 5B: graph showing tumor volume reduction in a human T-cell lymphoma xenograft model (e.g., Hh tumor cells) exposed to TRAC−/B2M−/CD70− anti-CD70 CAR+ T cells, e.g., CTX130 cells.

FIG. 6 is a schematic depicting an exemplary clinical study design to evaluate CTX130 cells administration to adult subjects with relapsed or refractory T cell or B cell malignancies. DLT: dose-limiting toxicity; M: month; max: maximum; min: minimum. The DLT evaluation period is the first 28 days after CTX130 infusion.

The details of one or more embodiments of the invention are set forth in the description below. Other features or advantages of the present invention will be apparent from the following drawings and detailed description of several embodiments, and also from the appended claims.

### DETAILED DESCRIPTION

CD70 is a type II membrane protein and ligand for the tumor necrosis factor receptor (TNFR) superfamily member CD27 with a healthy tissue expression distribution limited to activated lymphocytes and subsets of dendritic and thymic epithelial cells and in both humans and mice.

In contrast to its tightly controlled normal tissue expression, CD70 is commonly expressed at elevated levels in multiple T cell and B cell malignancies including peripheral T cell lymphoma not otherwise specified (PTCL-NOS), anaplastic large cell lymphoma (ALCL), Sézary syndrome (SS) including mycosis fungoides (MF), non-smoldering acute adult T cell leukemia/lymphoma (ATLL), angioimmunoblastic T cell lymphoma (AITL; also known as PTCL-AITL), and diffuse large B cell lymphoma (DLBCL). CD70 is also expressed in other hematopoietic malignancies such as myeloid malignancies.

Although hematopoietic cell malignancies such as T cell and B cell malignancies may be treated using conventional treatments, such as chemotherapy and/or checkpoint inhibitors (CPIs), patients may respond poorly or not at all, or relapse after treatment. Such patients have no treatment options with established life-prolonging benefit and are in need of new treatment alternatives.

Surprisingly, the anti-CD70 CAR+ T cells disclosed herein such as CTX130 cells successfully reduced tumor burden in a subcutaneous T cell lymphoma xenograft model and displayed long-term in vivo efficacy that eliminated tumor growth for an extended period (e.g., 90 days after treatment).

Accordingly, the present disclosure provides, in some aspects, therapeutic uses of anti-CD70 CAR+ T cells (e.g., CTX130 cells) for treating T cell, B cell, and myeloid cell malignancies. The anti-CD70 CAR T cells, methods of producing such (e.g., via the CRISPR approach), as well as components and processes (e.g., the CRISPR approach for gene editing and components used therein) for making the anti-CD70 CAR+ T cells disclosed herein are also within the scope of the present disclosure.

### I. Anti-CD70 Allogeneic CAR T Cells

Disclosed herein are anti-CD70 CAR T cells (e.g., CTX130 cells) for use in treating a hematopoietic cell malignancy, such as a T cell malignancy, a B cell malignancy, or a myeloid cell malignancy. In some embodiments, the anti-CD70 CAR T cells are allogeneic T cells having a disrupted TRAC gene, a disrupted B2M gene, a disrupted CD70 gene, or a combination thereof. In specific examples, the anti-CD70 CAR T cells express an anti-CD70 CAR and have endogenous TRAC, B2M, and CD70 genes disrupted. Any suitable gene editing methods known in the art can be used for making the anti-CD70 CAR T cells disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9).

Exemplary genetic modifications of the anti-CD70 CAR T cells include targeted disruption of T cell receptor alpha constant (TRAC), β2M, CD70, or a combination thereof. The disruption of the TRAC locus results in loss of expression of the T cell receptor (TCR) and is intended to reduce the probability of Graft versus Host Disease (GvHD), while the disruption of the β2M locus results in lack of expression of the major histocompatibility complex type I (MHC I) proteins and is intended to improve persistence by reducing the probability of host rejection. The disruption of CD70 results in loss of expression of CD70, which prevents possible cell-to-cell fratricide prior to insertion of the CD70 CAR. The addition of the anti-CD70 CAR directs the modified T cells towards CD70-expressing tumor cells.

The anti-CD70 CAR may comprise an anti-CD70 single-chain variable fragment (scFv) specific for CD70, followed by hinge domain and transmembrane domain (e.g., a CD8 hinge and transmembrane domain) that is fused to an intracellular co-signaling domain (e.g., a 4-1BB co-stimulatory domain) and a CD3ζ signaling domain.

### (i) Chimeric Antigen Receptor (CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include MLLLVTSLLLCELPHPAFLLIP (SEQ ID NO: 52) and MALPVTALLLPLALLLHAARP (SEQ ID NO: 53). Other signal peptides may be used.

(a) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain may be specific to a target antigen of interest, for example, a pathologic antigen such as a tumor antigen. In some embodiments, a tumor antigen is a “tumor associated antigen,” referring to an immunogenic molecule, such as a protein, that is generally expressed at a higher level in tumor cells than in non-tumor cells, in which it may not be expressed at all, or only at low levels. In some embodiments, tumor-associated structures, which are recognized by the immune system of the tumor-harboring host, are referred to as tumor-associated antigens. In some embodiments, a tumor-associated antigen is a universal tumor antigen, if it is broadly expressed by most types of tumors. In some embodiments, tumor-associated antigens are differentiation antigens, mutational antigens, overexpressed cellular antigens or viral antigens. In some embodiments, a tumor antigen is a “tumor specific antigen” or “TSA,” referring to an immunogenic molecule, such as a protein, that is unique to a tumor cell. Tumor specific antigens are exclusively expressed in tumor cells, for example, in a specific type of tumor cells.

In some examples, the CAR constructs disclosed herein comprise a scFv extracellular domain capable of binding to CD70. An example of an anti-CD70 CAR is provided in Examples below.

(b) Transmembrane Domain

The CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a transmembrane domain containing the sequence of FVPVFLPAKPTTTPAPRPPTPAPTIAS QPLSLRPEACRPAAGG AVHTRGLDFACDIYIWAPLAGTCGVLLLSLVITLYCNHRNR (SEQ ID NO: 54) or IYIWAPLAGTCGVLLLSLVITLY (SEQ ID NO: 55). Other transmembrane domains may be used.

(c) Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.

(d) Intracellular Signaling Domains

Any of the CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

It should be understood that methods described herein encompasses more than one suitable CAR that can be used to produce genetically engineered T cells expressing the CAR, for example, those known in the art or disclosed herein. Examples can be found in, e.g., WO 2019/097305A2, and WO2019/215500, the relevant disclosures of each of the prior applications are incorporated by reference herein for the purpose and subject matter referenced herein.

For example, the CAR binds CD70 (also known as a “CD70 CAR” or an “anti-CD70 CAR”). The amino acid sequence of an exemplary CAR that binds CD70 is provided in SEQ ID NO: 46. See also amino acid sequences and coding nucleotide sequences of components in an exemplary anti-CD70 CAR construct in Table 1 below.

TABLE 1
Sequences of Exemplary Anti-CD70 CAR Construct Components.

| | | SEQ ID |
|---|---|---|
| Description | Sequence | NO: |
| CD70 | CCTGCAGGCAGCTGCGCGCTCGCTCGCTCACTGAGGCCGCCCGGGCGTCGG | 43 |
| rAAV | GCGACCTTTGGTCGCCCGGCCTCAGTGAGCGAGCGAGCGCGCAGAGAGGGA | |
| (CD70B scFV | GTGGCCAACTCCATCACTAGGGGTTCCTGCGGCCGCACGCGTGAGATGTAA | |
| with 41BB) | GGAGCTGCTGTGACTTGCTCAAGGCCTTATATCGAGTAAACGGTAGTGCTG | |
| | GGGCTTAGACGCAGGTGTTCTGATTTATAGTTCAAAACCTCTATCAATGAG | |
| | AGAGCAATCTCCTGGTAATGTGATAGATTTCCCAACTTAATGCCAACATAC | |
| | CATAAACCTCCCATTCTGCTAATGCCCAGCCTAAGTTGGGGAGACCACTCC | |
| | AGATTCCAAGATGTACAGTTTGCTTTGCTGGGCCTTTTTCCCATGCCTGCC | |
| | TTTACTCTGCCAGAGTTATATTGCTGGGGTTTTGAAGAAGATCCTATTAAA | |
| | TAAAAGAATAAGCAGTATTATTAAGTAGCCCTGCATTTCAGGTTTCCTTGA | |
| | GTGGCAGGCCAGGCCTGGCCGTGAACGTTCACTGAAATCATGGCCTCTTGG | |
| | CCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCC | |
| | AGCCCCACAGAGCCCCGCCCTTGTCCATCACTGGCATCTGGACTCCAGCCT | |
| | GGGTTGGGGCAAAGAGGGAAATGAGATCATGTCCTAACCCTGATCCTCTTG | |
| | TCCCACAGATATCCAGAACCCTGACCCTGCCGTGTACCAGCTGAGAGACTC | |
| | TAAATCCAGTGACAAGTCTGTCTGCCTATTCACCGATTTTGATTCTCAAAC | |
| | AAATGTGTCACAAAGTAAGGATTCTGATGTGTATATCACAGACAAAACTGT | |
| | GCTAGACATGAGGTCTATGGACTTCAGGCTCCGGTGCCCGTCAGTGGGCAG | |
| | AGCGCACATCGCCCACAGTCCCCGAGAAGTTGGGGGGAGGGGTCGGCAATT | |
| | GAACCGGTGCCTAGAGAAGGTGGCGCGGGGTAAACTGGGAAAGTGATGTCG | |
| | TGTACTGGCTCCGCCTTTTTCCCGAGGGTGGGGGAGAACCGTATATAAGTG | |
| | CAGTAGTCGCCGTGAACGTTCTTTTTCGCAACGGGTTTGCCGCCAGAACAC | |
| | AGGTAAGTGCCGTGTGTGGTTCCCGCGGGCCTGGCCTCTTTACGGGTTATG | |
| | GCCCTTGCGTGCCTTGAATTACTTCCACTGGCTGCAGTACGTGATTCTTGA | |
| | TCCCGAGCTTCGGGTTGGAAGTGGGTGGGAGAGTTCGAGGCCTTGCGCTTA | |
| | AGGAGCCCCTTCGCCTCGTGCTTGAGTTGAGGCCTGGCCTGGGCGCTGGGG | |
| | CCGCCGCGTGCGAATCTGGTGGCACCTTCGCGCCTGTCTCGCTGCTTTCGA | |
| | TAAGTCTCTAGCCATTTAAAATTTTTGATGACCTGCTGCGACGCTTTTTTT | |
| | CTGGCAAGATAGTCTTGTAAATGCGGGCCAAGATCTGCACACTGGTATTTC | |
| | GGTTTTTGGGGCCGCGGGCGGCGACGGGGCCCGTGCGTCCCAGCGCACATG | |
| | TTCGGCGAGGCGGGGCCTGCGAGCGCGGCCACCGAGAATCGGACGGGGGTA | |
| | GTCTCAAGCTGGCCGGCCTGCTCTGGTGCCTGGCCTCGCGCCGCCGTGTAT | |
| | CGCCCCGCCCTGGGCGGCAAGGCTGGCCCGGTCGGCACCAGTTGCGTGAGC | |
| | GGAAAGATGGCCGCTTCCCGGCCCTGCTGCAGGGAGCTCAAAATGGAGGAC | |
| | GCGGCGCTCGGGAGAGCGGGCGGGTGAGTCACCCACACAAAGGAAAAGGGC | |
| | CTTTCCGTCCTCAGCCGTCGCTTCATGTGACTCCACGGAGTACCGGGCGCC | |
| | GTCCAGGCACCTCGATTAGTTCTCGAGCTTTTGGAGTACGTCGTCTTTAGG | |
| | TTGGGGGGAGGGGTTTTATGCGATGGAGTTTCCCCACACTGAGTGGGTGGA | |
| | GACTGAAGTTAGGCCAGCTTGGCACTTGATGTAATTCTCCTTGGAATTTGC | |
| | CCTTTTTGAGTTTGGATCTTGGTTCATTCTCAAGCCTCAGACAGTGGTTCA | |
| | AAGTTTTTTTCTTCCATTTCAGGTGTCGTGACCACCATGGCGCTTCCGGTG | |
| | ACAGCACTGCTCCTCCCCTTGGCGCTGTTGCTCCACGCAGCAAGGCCGCAG | |
| | GTCCAGTTGGTGCAAAGCGGGGCGGAGGTGAAAAAACCCGGCGCTTCCGTG | |
| | AAGGTGTCCTGTAAGGCGTCCGGTTATACGTTCACGAACTACGGGATGAAT | |
| | TGGGTTCGCCAAGCGCCGGGGCAGGGACTGAAATGGATGGGGTGGATAAAT | |
| | ACCTACACCGGCGAACCTACATACGCCGACGCTTTTAAAGGGCGAGTCACT | |
| | ATGACGCGCGATACCAGCATATCCACCGCATACATGGAGCTGTCCCGACTC | |
| | CGGTCAGACGACACGGCTGTCTACTATTGTGCTCGGGACTATGGCGATTAT | |
| | GGCATGGACTACTGGGGTCAGGGTACGACTGTAACAGTTAGTAGTGGTGGA | |
| | GGCGGCAGTGGCGGGGGGGGAAGCGGAGGAGGGGGTTCTGGTGACATAGTT | |
| | ATGACCCAATCCCCAGATAGTTTGGCGGTTTCTCTGGGCGAGAGGGCAACG | |
| | ATTAATTGTCGCGCATCAAAGAGCGTTTCAACGAGCGGATATTCTTTTATG | |
| | CATTGGTACCAGCAAAAACCCGGACAACCGCCGAAGCTGCTGATCTACTTG | |
| | GCTTCAAATCTTGAGTCTGGGGTGCCGGACCGATTTTCTGGTAGTGGAAGC | |
| | GGAACTGACTTTACGCTCACGATCAGTTCACTGCAGGCTGAGGATGTAGCG | |
| | GTCTATTATTGCCAGCACAGTAGAGAAGTCCCCTGGACCTTCGGTCAAGGC | |
| | ACGAAAGTAGAAATTAAAAGTGCTGCTGCCTTTGTCCCGGTATTTCTCCCA | |
| | GCCAAACCGACCACGACTCCCGCCCCGCGCCCTCCGACACCCGCTCCCACC | |
| | ATCGCCTCTCAACCTCTTAGTCTTCGCCCCGAGGCATGCCGACCCGCCGCC | |
| | GGGGGTGCTGTTCATACGAGGGGCTTGGACTTCGCTTGTGATATTTACATT | |
| | TGGGCTCCGTTGGCGGGTACGTGCGGCGTCCTTTTGTTGTCACTCGTTATT | |
| | ACTTTGTATTGTAATCACAGGAATCGCAAACGGGGCAGAAAGAAACTCCTG | |
| | TATATATTCAAACAACCATTTATGAGACCAGTACAAACTACTCAAGAGGAA | |
| | GATGGCTGTAGCTGCCGATTTCCAGAAGAAGAAGAAGGAGGATGTGAACTG | |
| | CGAGTGAAGTTTTCCCGAAGCGCAGACGCTCCGGCATATCAGCAAGGACAG | |
| | AATCAGCTGTATAACGAACTGAATTTGGGACGCCGCGAGGAGTATGACGTG | |
| | CTTGATAAACGCCGGGGGAGAGACCCGGAAATGGGGGGTAAACCCCGAAGA | |
| | AAGAATCCCCAAGAAGGACTCTACAATGAACTCCAGAAGGATAAGATGGCG | |
| | GAGGCCTACTCAGAAATAGGTATGAAGGGCGAACGACGACGGGGAAAAGGT | |
| | CACGATGGCCTCTACCAAGGGTTGAGTACGGCAACCAAAGATACGTACGAT | |
| | GCACTGCATATGCAGGCCCTGCCTCCCAGATAATAATAAAATCGCTATCCA | |
| | TCGAAGATGGATGTGTGTTGGTTTTTTGTGTGTGGAGCAACAAATCTGACT | |
| | TTGCATGTGCAAACGCCTTCAACAACAGCATTATTCCAGAAGACACCTTCT | |
| | TCCCCAGCCCAGGTAAGGGCAGCTTTGGTGCCTTCGCAGGCTGTTTCCTTG | |
| | CTTCAGGAATGGCCAGGTTCTGCCCAGAGCTCTGGTCAATGATGTCTAAAA | |
| | CTCCTCTGATTGGTGGTCTCGGCCTTATCCATTGCCACCAAAACCCTCTTT | |
| | TTACTAAGAAACAGTGAGCCTTGTTCTGGCAGTCCAGAGAATGACACGGGA | |
| | AAAAAGCAGATGAAGAGAAGGTGGCAGGAGAGGGCACGTGGCCCAGCCTCA | |
| | GTCTCTCCAACTGAGTTCCTGCCTGCCTGCCTTTGCTCAGACTGTTTGCCC | |
| | CTTACTGCTCTTCTAGGCCTCATTCTAAGCCCCTTCTCCAAGTTGCCTCTC | |
| | CTTATTTCTCCCTGTCTGCCAAAAAATCTTTCCCAGCTCACTAAGTCAGTC | |
| | TCACGCAGTCACTCATTAACCCACCAATCACTGATTGTGCCGGCACATGAA | |
| | TGCACCAGGTGTTGAAGTGGAGGAATTAAAAAGTCAGATGAGGGGTGTGCC | |
| | CAGAGGAAGCACCATTCTAGTTGGGGGAGCCCATCTGTCAGCTGGGAAAAG | |
| | TCCAAATAACTTCAGATTGGAATGTGTTTTAACTCAGGGTTGAGAAAACAG | |
| | CTACCTTCAGGACAAAAGTCAGGGAAGGGCTCTCTGAAGAAATGCTACTTG | |
| | AAGATACCAGCCCTACCAAGGGCAGGGAGAGGACCCTATAGAGGCCTGGGA | |
| | CAGGAGCTCAATGAGAAAGGTAACCACGTGCGGACCGAGGCTGCAGCGTCG | |
| | TCCTCCCTAGGAACCCCTAGTGATGGAGTTGGCCACTCCCTCTCTGCGCGC | |
| | TCGCTCGCTCACTGAGGCCGGGCGACCAAAGGTCGCCCGACGCCCGGGCTT | |
| | TGCCCGGGCGGCCTCAGTGAGCGAGCGAGCGCGCAGCTGCCTGCAGG | |
| CD70 | GAGATGTAAGGAGCTGCTGTGACTTGCTCAAGGCCTTATATCGAGTAAACG | 44 |
| LHA to RHA | GTAGTGCTGGGGCTTAGACGCAGGTGTTCTGATTTATAGTTCAAAACCTCT | |
| (CD70B ScFV | ATCAATGAGAGAGCAATCTCCTGGTAATGTGATAGATTTCCCAACTTAATG | |
| with 41BB) | CCAACATACCATAAACCTCCCATTCTGCTAATGCCCAGCCTAAGTTGGGGA | |
| | GACCACTCCAGATTCCAAGATGTACAGTTTGCTTTGCTGGGCCTTTTTCCC | |
| | ATGCCTGCCTTTACTCTGCCAGAGTTATATTGCTGGGGTTTTGAAGAAGAT | |
| | CCTATTAAATAAAAGAATAAGCAGTATTATTAAGTAGCCCTGCATTTCAGG | |
| | TTTCCTTGAGTGGCAGGCCAGGCCTGGCCGTGAACGTTCACTGAAATCATG | |
| | GCCTCTTGGCCAAGATTGATAGCTTGTGCCTGTCCCTGAGTCCCAGTCCAT | |
| | CACGAGCAGCTGGTTTCTAAGATGCTATTTCCCGTATAAAGCATGAGACCG | |
| | TGACTTGCCAGCCCCACAGAGCCCCGCCCTTGTCCATCACTGGCATCTGGA | |
| | CTCCAGCCTGGGTTGGGGCAAAGAGGGAAATGAGATCATGTCCTAACCCTG | |
| | ATCCTCTTGTCCCACAGATATCCAGAACCCTGACCCTGCCGTGTACCAGCT | |
| | GAGAGACTCTAAATCCAGTGACAAGTCTGTCTGCCTATTCACCGATTTTGA | |
| | TTCTCAAACAAATGTGTCACAAAGTAAGGATTCTGATGTGTATATCACAGA | |
| | CAAAACTGTGCTAGACATGAGGTCTATGGACTTCAGGCTCCGGTGCCCGTC | |
| | AGTGGGCAGAGCGCACATCGCCCACAGTCCCCGAGAAGTTGGGGGGAGGGG | |
| | TCGGCAATTGAACCGGTGCCTAGAGAAGGTGGCGCGGGGTAAACTGGGAAA | |
| | GTGATGTCGTGTACTGGCTCCGCCTTTTTCCCGAGGGTGGGGGAGAACCGT | |
| | ATATAAGTGCAGTAGTCGCCGTGAACGTTCTTTTTCGCAACGGGTTTGCCG | |
| | CCAGAACACAGGTAAGTGCCGTGTGTGGTTCCCGCGGGCCTGGCCTCTTTA | |
| | CGGGTTATGGCCCTTGCGTGCCTTGAATTACTTCCACTGGCTGCAGTACGT | |
| | GATTCTTGATCCCGAGCTTCGGGTTGGAAGTGGGTGGGAGAGTTCGAGGCC | |
| | TTGCGCTTAAGGAGCCCCTTCGCCTCGTGCTTGAGTTGAGGCCTGGCCTGG | |
| | GCGCTGGGGCCGCCGCGTGCGAATCTGGTGGCACCTTCGCGCCTGTCTCGC | |
| | TGCTTTCGATAAGTCTCTAGCCATTTAAAATTTTTGATGACCTGCTGCGAC | |
| | GCTTTTTTTCTGGCAAGATAGTCTTGTAAATGCGGGCCAAGATCTGCACAC | |
| | TGGTATTTCGGTTTTTGGGGCCGCGGGCGGCGACGGGGCCCGTGCGTCCCA | |
| | GCGCACATGTTCGGCGAGGCGGGGCCTGCGAGCGCGGCCACCGAGAATCGG | |
| | ACGGGGGTAGTCTCAAGCTGGCCGGCCTGCTCTGGTGCCTGGCCTCGCGCC | |
| | GCCGTGTATCGCCCCGCCCTGGGCGGCAAGGCTGGCCCGGTCGGCACCAGT | |
| | TGCGTGAGCGGAAAGATGGCCGCTTCCCGGCCCTGCTGCAGGGAGCTCAAA | |
| | ATGGAGGACGCGGCGCTCGGGAGAGCGGGCGGGTGAGTCACCCACACAAAG | |
| | GAAAAGGGCCTTTCCGTCCTCAGCCGTCGCTTCATGTGACTCCACGGAGTA | |
| | CCGGGCGCCGTCCAGGCACCTCGATTAGTTCTCGAGCTTTTGGAGTACGTC | |
| | GTCTTTAGGTTGGGGGGAGGGGTTTTATGCGATGGAGTTTCCCCACACTGA | |
| | GTGGGTGGAGACTGAAGTTAGGCCAGCTTGGCACTTGATGTAATTCTCCTT | |
| | GGAATTTGCCCTTTTTGAGTTTGGATCTTGGTTCATTCTCAAGCCTCAGAC | |
| | AGTGGTTCAAAGTTTTTTTCTTCCATTTCAGGTGTCGTGACCACCATGGCG | |
| | CTTCCGGTGACAGCACTGCTCCTCCCCTTGGCGCTGTTGCTCCACGCAGCA | |
| | AGGCCGCAGGTCCAGTTGGTGCAAAGCGGGGCGGAGGTGAAAAAACCCGGC | |
| | GCTTCCGTGAAGGTGTCCTGTAAGGCGTCCGGTTATACGTTCACGAACTAC | |
| | GGGATGAATTGGGTTCGCCAAGCGCCGGGGCAGGGACTGAAATGGATGGGG | |
| | TGGATAAATACCTACACCGGCGAACCTACATACGCCGACGCTTTTAAAGGG | |
| | CGAGTCACTATGACGCGCGATACCAGCATATCCACCGCATACATGGAGCTG | |
| | TCCCGACTCCGGTCAGACGACACGGCTGTCTACTATTGTGCTCGGGACTAT | |
| | GGCGATTATGGCATGGACTACTGGGGTCAGGGTACGACTGTAACAGTTAGT | |
| | AGTGGTGGAGGCGGCAGTGGCGGGGGGGGAAGCGGAGGAGGGGGTTCTGGT | |
| | GACATAGTTATGACCCAATCCCCAGATAGTTTGGCGGTTTCTCTGGGCGAG | |
| | AGGGCAACGATTAATTGTCGCGCATCAAAGAGCGTTTCAACGAGCGGATAT | |
| | TCTTTTATGCATTGGTACCAGCAAAAACCCGGACAACCGCCGAAGCTGCTG | |
| | ATCTACTTGGCTTCAAATCTTGAGTCTGGGGTGCCGGACCGATTTTCTGGT | |
| | AGTGGAAGCGGAACTGACTTTACGCTCACGATCAGTTCACTGCAGGCTGAG | |
| | GATGTAGCGGTCTATTATTGCCAGCACAGTAGAGAAGTCCCCTGGACCTTC | |
| | GGTCAAGGCACGAAAGTAGAAATTAAAAGTGCTGCTGCCTTTGTCCCGGTA | |
| | TTTCTCCCAGCCAAACCGACCACGACTCCCGCCCCGCGCCCTCCGACACCC | |
| | GCTCCCACCATCGCCTCTCAACCTCTTAGTCTTCGCCCCGAGGCATGCCGA | |
| | CCCGCCGCCGGGGGTGCTGTTCATACGAGGGGCTTGGACTTCGCTTGTGAT | |
| | ATTTACATTTGGGCTCCGTTGGCGGGTACGTGCGGCGTCCTTTTGTTGTCA | |
| | CTCGTTATTACTTTGTATTGTAATCACAGGAATCGCAAACGGGGCAGAAAG | |
| | AAACTCCTGTATATATTCAAACAACCATTTATGAGACCAGTACAAACTACT | |
| | CAAGAGGAAGATGGCTGTAGCTGCCGATTTCCAGAAGAAGAAGAAGGAGGA | |
| | TGTGAACTGCGAGTGAAGTTTTCCCGAAGCGCAGACGCTCCGGCATATCAG | |
| | CAAGGACAGAATCAGCTGTATAACGAACTGAATTTGGGACGCCGCGAGGAG | |
| | TATGACGTGCTTGATAAACGCCGGGGGAGAGACCCGGAAATGGGGGGTAAA | |
| | CCCCGAAGAAAGAATCCCCAAGAAGGACTCTACAATGAACTCCAGAAGGAT | |
| | AAGATGGCGGAGGCCTACTCAGAAATAGGTATGAAGGGCGAACGACGACGG | |
| | GGAAAAGGTCACGATGGCCTCTACCAAGGGTTGAGTACGGCAACCAAAGAT | |
| | ACGTACGATGCACTGCATATGCAGGCCCTGCCTCCCAGATAATAATAAAAT | |
| | CGCTATCCATCGAAGATGGATGTGTGTTGGTTTTTTGTGTGTGGAGCAACA | |
| | AATCTGACTTTGCATGTGCAAACGCCTTCAACAACAGCATTATTCCAGAAG | |
| | ACACCTTCTTCCCCAGCCCAGGTAAGGGCAGCTTTGGTGCCTTCGCAGGCT | |
| | GTTTCCTTGCTTCAGGAATGGCCAGGTTCTGCCCAGAGCTCTGGTCAATGA | |
| | TGTCTAAAACTCCTCTGATTGGTGGTCTCGGCCTTATCCATTGCCACCAAA | |
| | ACCCTCTTTTTACTAAGAAACAGTGAGCCTTGTTCTGGCAGTCCAGAGAAT | |
| | GACACGGGAAAAAAGCAGATGAAGAGAAGGTGGCAGGAGAGGGCACGTGGC | |
| | CCAGCCTCAGTCTCTCCAACTGAGTTCCTGCCTGCCTGCCTTTGCTCAGAC | |
| | TGTTTGCCCCTTACTGCTCTTCTAGGCCTCATTCTAAGCCCCTTCTCCAAG | |
| | TTGCCTCTCCTTATTTCTCCCTGTCTGCCAAAAAATCTTTCCCAGCTCACT | |
| | AAGTCAGTCTCACGCAGTCACTCATTAACCCACCAATCACTGATTGTGCCG | |
| | GCACATGAATGCACCAGGTGTTGAAGTGGAGGAATTAAAAAGTCAGATGAG | |
| | GGGTGTGCCCAGAGGAAGCACCATTCTAGTTGGGGGAGCCCATCTGTCAGC | |
| | TGGGAAAAGTCCAAATAACTTCAGATTGGAATGTGTTTTAACTCAGGGTTG | |
| | AGAAAACAGCTACCTTCAGGACAAAAGTCAGGGAAGGGCTCTCTGAAGAAA | |
| | TGCTACTTGAAGATACCAGCCCTACCAAGGGCAGGGAGAGGACCCTATAGA | |
| | GGCCTGGGACAGGAGCTCAATGAGAAAGG | |
| CD70 CAR | ATGGCGCTTCCGGTGACAGCACTGCTCCTCCCCTTGGCGCTGTTGCTCCACG | 45 |
| nucleotide | CAGCAAGGCCGCAGGTCCAGTTGGTGCAAAGCGGGGCGGAGGTGAAAAAACC | |
| sequence | CGGCGCTTCCGTGAAGGTGTCCTGTAAGGCGTCCGGTTATACGTTCACGAAC | |
| (CD70B scFv | TACGGGATGAATTGGGTTCGCCAAGCGCCGGGGCAGGGACTGAAATGGATGG | |
| with 41BB) | GGTGGATAAATACCTACACCGGCGAACCTACATACGCCGACGCTTTTAAAGG | |
| | GCGAGTCACTATGACGCGCGATACCAGCATATCCACCGCATACATGGAGCTG | |
| | TCCCGACTCCGGTCAGACGACACGGCTGTCTACTATTGTGCTCGGGACTATG | |
| | GCGATTATGGCATGGACTACTGGGGTCAGGGTACGACTGTAACAGTTAGTAG | |
| | TGGTGGAGGCGGCAGTGGCGGGGGGGGAAGCGGAGGAGGGGGTTCTGGTGAC | |
| | ATAGTTATGACCCAATCCCCAGATAGTTTGGCGGTTTCTCTGGGCGAGAGGG | |
| | CAACGATTAATTGTCGCGCATCAAAGAGCGTTTCAACGAGCGGATATTCTTT | |
| | TATGCATTGGTACCAGCAAAAACCCGGACAACCGCCGAAGCTGCTGATCTAC | |
| | TTGGCTTCAAATCTTGAGTCTGGGGTGCCGGACCGATTTTCTGGTAGTGGAA | |
| | GCGGAACTGACTTTACGCTCACGATCAGTTCACTGCAGGCTGAGGATGTAGC | |
| | GGTCTATTATTGCCAGCACAGTAGAGAAGTCCCCTGGACCTTCGGTCAAGGC | |
| | ACGAAAGTAGAAATTAAAAGTGCTGCTGCCTTTGTCCCGGTATTTCTCCCAG | |
| | CCAAACCGACCACGACTCCCGCCCCGCGCCCTCCGACACCCGCTCCCACCAT | |
| | CGCCTCTCAACCTCTTAGTCTTCGCCCCGAGGCATGCCGACCCGCCGCCGGG | |
| | GGTGCTGTTCATACGAGGGGCTTGGACTTCGCTTGTGATATTTACATTTGGG | |
| | CTCCGTTGGCGGGTACGTGCGGCGTCCTTTTGTTGTCACTCGTTATTACTTT | |
| | GTATTGTAATCACAGGAATCGCAAACGGGGCAGAAAGAAACTCCTGTATATA | |
| | TTCAAACAACCATTTATGAGACCAGTACAAACTACTCAAGAGGAAGATGGCT | |
| | GTAGCTGCCGATTTCCAGAAGAAGAAGAAGGAGGATGTGAACTGCGAGTGAA | |
| | GTTTTCCCGAAGCGCAGACGCTCCGGCATATCAGCAAGGACAGAATCAGCTG | |
| | TATAACGAACTGAATTTGGGACGCCGCGAGGAGTATGACGTGCTTGATAAAC | |
| | GCCGGGGGAGAGACCCGGAAATGGGGGGTAAACCCCGAAGAAAGAATCCCCA | |
| | AGAAGGACTCTACAATGAACTCCAGAAGGATAAGATGGCGGAGGCCTACTCA | |
| | GAAATAGGTATGAAGGGCGAACGACGACGGGGAAAAGGTCACGATGGCCTCT | |
| | ACCAAGGGTTGAGTACGGCAACCAAAGATACGTACGATGCACTGCATATGCA | |
| | GGCCCTGCCTCCCAGATAA | |
| CD70 CAR amino | MALPVTALLLPLALLLHAARPQVQLVQSGAEVKKPGASVKVSCKASGYTFTN | 46 |
| acid sequence | YGMNWVRQAPGQGLKWMGWINTYTGEPTYADAFKGRVTMTRDTSISTAYMEL | |
| (CD70B scFv | SRLRSDDTAVYYCARDYGDYGMDYWGQGTTVTVSSGGGGSGGGGSGGGGSGD | |
| with 41BB) | IVMTQSPDSLAVSLGERATINCRASKSVSTSGYSFMHWYQQKPGQPPKLLIY | |
| | LASNLESGVPDRFSGSGSGTDFTLTISSLQAEDVAVYYCQHSREVPWTFGQG | |
| | TKVEIKSAAAFVPVFLPAKPTTTPAPRPPTPAPTIASQPLSLRPEACRPAAG | |
| | GAVHTRGLDFACDIYIWAPLAGTCGVLLLSLVITLYCNHRNRKRGRKKLLYI | |
| | FKQPFMRPVQTTQEEDGCSCRFPEEEEGGCELRVKFSRSADAPAYQQGQNQL | |
| | YNELNLGRREEYDVLDKRRGRDPEMGGKPRRKNPQEGLYNELQKDKMAEAYS | |
| | EIGMKGERRRGKGHDGLYQGLSTATKDTYDALHMQALPPR | |
| CD70B | CAGGTCCAGTTGGTGCAAAGCGGGGCGGAGGTGAAAAAACCCGGCGCTTCCG | 47 |
| scFv nucleotide | TGAAGGTGTCCTGTAAGGCGTCCGGTTATACGTTCACGAACTACGGGATGAA | |
| sequence | TTGGGTTCGCCAAGCGCCGGGGCAGGGACTGAAATGGATGGGGTGGATAAAT | |
| | ACCTACACCGGCGAACCTACATACGCCGACGCTTTTAAAGGGCGAGTCACTA | |
| | TGACGCGCGATACCAGCATATCCACCGCATACATGGAGCTGTCCCGACTCCG | |
| | GTCAGACGACACGGCTGTCTACTATTGTGCTCGGGACTATGGCGATTATGGC | |
| | ATGGACTACTGGGGTCAGGGTACGACTGTAACAGTTAGTAGTGGTGGAGGCG | |
| | GCAGTGGCGGGGGGGGAAGCGGAGGAGGGGGTTCTGGTGACATAGTTATGAC | |
| | CCAATCCCCAGATAGTTTGGCGGTTTCTCTGGGCGAGAGGGCAACGATTAAT | |
| | TGTCGCGCATCAAAGAGCGTTTCAACGAGCGGATATTCTTTTATGCATTGGT | |
| | ACCAGCAAAAACCCGGACAACCGCCGAAGCTGCTGATCTACTTGGCTTCAAA | |
| | TCTTGAGTCTGGGGTGCCGGACCGATTTTCTGGTAGTGGAAGCGGAACTGAC | |
| | TTTACGCTCACGATCAGTTCACTGCAGGCTGAGGATGTAGCGGTCTATTATT | |
| | GCCAGCACAGTAGAGAAGTCCCCTGGACCTTCGGTCAAGGCACGAAAGTAGA | |
| | AATTAAA | |
| CD70B | QVQLVQSGAEVKKPGASVKVSCKASGYTFTNYGMNWVRQAPGQGLKWMGWIN | 48 |
| scFv amino | TYTGEPTYADAFKGRVTMTRDTSISTAYMELSRLRSDDTAVYYCARDYGDYG | |
| acid sequence | MDYWGQGTTVTVSSGGGGSGGGGSGGGGSGDIVMTQSPDSLAVSLGERATIN | |
| (linker | CRASKSVSTSGYSFMHWYQQKPGQPPKLLIYLASNLESGVPDRFSGSGSGTD | |
| underlined) | FTLTISSLQAEDVAVYYCQHSREVPWTFGQGTKVEIK | |
| CD70 V_{H} | QVQLVQSGAEVKKPGASVKVSCKASGYTFTNYGMNWVRQAPGQGLKWMGWIN | 49 |
| | TYTGEPTYADAFKGRVTMTRDTSISTAYMELSRLRSDDTAVYYCARDYGDYG | |
| | MDYWGQGTTVTVSS | |
| CD70 V_{L} | DIVMTQSPDSLAVSLGERATINCRASKSVSTSGYSFMHWYQQKPGQPPKLLI | 50 |
| | YLASNLESGVPDRFSGSGSGTDFTLTISSLQAEDVAVYYCQHSREVPWTFGQ | |
| | GTKVEIK | |
| Linker | GGGGSGGGGSGGGGSG | 51 |
| signal peptide | MLLLVTSLLLCELPHPAFLLIP | 52 |
| signal peptide | MALPVTALLLPLALLLHAARP | 53 |
| CD8a | FVPVFLPAKPTTTPAPRPPTPAPTIASQPLSLRPEACRPAAGGAVHTRGLDF | 54 |
| transmembrane | ACDIYIWAPLAGTCGVLLLSLVITLYCNHRNR | |
| domain | | |
| CD8a | IYIWAPLAGTCGVLLLSLVITLY | 55 |
| transmembrane | | |
| 4-1BB nucleotide | AAACGGGGCAGAAAGAAACTCCTGTATATATTCAAACAACCATTTATGAGAC | 56 |
| sequence | CAGTACAAACTACTCAAGAGGAAGATGGCTGTAGCTGCCGATTTCCAGAAGA | |
| | AGAAGAAGGAGGATGTGAACTG | |
| 4-1BB amino acid | KRGRKKLLYTFKQPFMRPVQTTQEEDGCSCRFPEEEEGGCEL | 57 |
| sequence | | |
| CD28 nucleotide | TCAAAGCGGAGTAGGTTGTTGCATTCCGATTACATGAATATGACTCCTCGCC | 58 |
| sequence | GGCCTGGGCCGACAAGAAAACATTACCAACCCTATGCCCCCCCACGAGACTT | |
| | CGCTGCGTACAGGTCC | |
| CD28 amino acid | SKRSRLLHSDYMNMTPRRPGPTRKHYQPYAPPRDFAAYRS | 59 |
| sequence | | |
| CD3ζ nucleotide | CGAGTGAAGTTTTCCCGAAGCGCAGACGCTCCGGCATATCAGCAAGGACAGA | 60 |
| sequence | ATCAGCTGTATAACGAACTGAATTTGGGACGCCGCGAGGAGTATGACGTGCT | |
| | TGATAAACGCCGGGGGAGAGACCCGGAAATGGGGGGTAAACCCCGAAGAAAG | |
| | AATCCCCAAGAAGGACTCTACAATGAACTCCAGAAGGATAAGATGGCGGAGG | |
| | CCTACTCAGAAATAGGTATGAAGGGCGAACGACGACGGGGAAAAGGTCACGA | |
| | TGGCCTCTACCAAGGGTTGAGTACGGCAACCAAAGATACGTACGATGCACTG | |
| | CATATGCAGGCCCTGCCTCCCAGA | |
| CD3ζ amino acid | RVKFSRSADAPAYQQGQNQLYNELNLGRREEYDVLDKRRGRDPEMGGKPRRK | 61 |
| sequence | NPQEGLYNELQKDKMAEAYSEIGMKGERRRGKGHDGLYQGLSTATKDTYDAL | |
| | HMQALPPR | |
| TRAC-LHA | GAGATGTAAGGAGCTGCTGTGACTTGCTCAAGGCCTTATATCGAGTAAACGG | 62 |
| | TAGTGCTGGGGCTTAGACGCAGGTGTTCTGATTTATAGTTCAAAACCTCTAT | |
| | CAATGAGAGAGCAATCTCCTGGTAATGTGATAGATTTCCCAACTTAATGCCA | |
| | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCA | |
| EF1α promoter | GGCTCCGGTGCCCGTCAGTGGGCAGAGCGCACATCGCCCACAGTCCCCGAGA | 63 |
| | AGTTGGGGGGAGGGGTCGGCAATTGAACCGGTGCCTAGAGAAGGTGGCGCGG | |
| | GGTAAACTGGGAAAGTGATGTCGTGTACTGGCTCCGCCTTTTTCCCGAGGGT | |
| | GGGGGAGAACCGTATATAAGTGCAGTAGTCGCCGTGAACGTTCTTTTTCGCA | |
| | ACGGGTTTGCCGCCAGAACACAGGTAAGTGCCGTGTGTGGTTCCCGCGGGCC | |
| | TGGCCTCTTTACGGGTTATGGCCCTTGCGTGCCTTGAATTACTTCCACTGGC | |
| | TGCAGTACGTGATTCTTGATCCCGAGCTTCGGGTTGGAAGTGGGTGGGAGAG | |
| | TTCGAGGCCTTGCGCTTAAGGAGCCCCTTCGCCTCGTGCTTGAGTTGAGGCC | |
| | TGGCCTGGGCGCTGGGGCCGCCGCGTGCGAATCTGGTGGCACCTTCGCGCCT | |
| | GTCTCGCTGCTTTCGATAAGTCTCTAGCCATTTAAAATTTTTGATGACCTGC | |
| | TGCGACGCTTTTTTTCTGGCAAGATAGTCTTGTAAATGCGGGCCAAGATCTG | |
| | CACACTGGTATTTCGGTTTTTGGGGCCGCGGGCGGCGACGGGGCCCGTGCGT | |
| | CCCAGCGCACATGTTCGGCGAGGCGGGGCCTGCGAGCGCGGCCACCGAGAAT | |
| | CGGACGGGGGTAGTCTCAAGCTGGCCGGCCTGCTCTGGTGCCTGGCCTCGCG | |
| | CCGCCGTGTATCGCCCCGCCCTGGGCGGCAAGGCTGGCCCGGTCGGCACCAG | |
| | TTGCGTGAGCGGAAAGATGGCCGCTTCCCGGCCCTGCTGCAGGGAGCTCAAA | |
| | ATGGAGGACGCGGCGCTCGGGAGAGCGGGCGGGTGAGTCACCCACACAAAGG | |
| | AAAAGGGCCTTTCCGTCCTCAGCCGTCGCTTCATGTGACTCCACGGAGTACC | |
| | GGGCGCCGTCCAGGCACCTCGATTAGTTCTCGAGCTTTTGGAGTACGTCGTC | |
| | TTTAGGTTGGGGGGAGGGGTTTTATGCGATGGAGTTTCCCCACACTGAGTGG | |
| | GTGGAGACTGAAGTTAGGCCAGCTTGGCACTTGATGTAATTCTCCTTGGAAT | |
| | TTGCCCTTTTTGAGTTTGGATCTTGGTTCATTCTCAAGCCTCAGACAGTGGT | |
| | TCAAAGTTTTTTTCTTCCATTTCAGGTGTCGTGA | |
| Synthetic poly(A) | AATAAAATCGCTATCCATCGAAGATGGATGTGTGTTGGTTTTTTGTGTG | 64 |
| signal | | |
| TRAC-RHA | TGGAGCAACAAATCTGACTTTGCATGTGCAAACGCCTTCAACAACAGCATTA | 65 |
| | TTCCAGAAGACACCTTCTTCCCCAGCCCAGGTAAGGGCAGCTTTGGTGCCTT | |
| | CGCAGGCTGTTTCCTTGCTTCAGGAATGGCCAGGTTCTGCCCAGAGCTCTGG | |
| | TCAATGATGTCTAAAACTCCTCTGATTGGTGGTCTCGGCCTTATCCATTGCC | |
| | ACCAAAACCCTCTTTTTACTAAGAAACAGTGAGCCTTGTTCTGGCAGTCCAG | |
| | AGAATGACACGGGAAAAAAGCAGATGAAGAGAAGGTGGCAGGAGAGGGCACG | |
| | TGGCCCAGCCTCAGTCTCTCCAACTGAGTTCCTGCCTGCCTGCCTTTGCTCA | |
| | GACTGTTTGCCCCTTACTGCTCTTCTAGGCCTCATTCTAAGCCCCTTCTCCA | |
| | AGTTGCCTCTCCTTATTTCTCCCTGTCTGCCAAAAAATCTTTCCCAGCTCAC | |
| | TAAGTCAGTCTCACGCAGTCACTCATTAACCCACCAATCACTGATTGTGCCG | |
| | GCACATGAATGCACCAGGTGTTGAAGTGGAGGAATTAAAAAGTCAGATGAGG | |
| | GGTGTGCCCAGAGGAAGCACCATTCTAGTTGGGGGAGCCCATCTGTCAGCTG | |
| | GGAAAAGTCCAAATAACTTCAGATTGGAATGTGTTTTAACTCAGGGTTGAGA | |
| | AAACAGCTACCTTCAGGACAAAAGTCAGGGAAGGGCTCTCTGAAGAAATGCT | |
| | ACTTGAAGATACCAGCCCTACCAAGGGCAGGGAGAGGACCCTATAGAGGCCT | |
| | GGGACAGGAGCTCAATGAGAAAGG | |

(ii) Knock-Out of TRAC, B2M, and/or CD70 Genes

The anti-CD70 CAR-T cells disclosed herein may further have a disrupted TRAC gene, a disrupted B2M gene, a disrupted CD70 gene, or a combination thereof. The disruption of the TRAC locus results in loss of expression of the T cell receptor (TCR) and is intended to reduce the probability of Graft versus Host Disease (GvHD), while the disruption of the β2M locus results in lack of expression of the major histocompatibility complex type I (MHC I) proteins and is intended to improve persistence by reducing the probability of host rejection. The disruption of the CD70 gene would minimize the fratricide effect in producing the anti-CD70 CAR-T cells. Further, disruption of the CD70 gene unexpectedly increased healthy and activity of the resultant engineered T cells. The addition of the anti-CD70 CAR directs the modified T cells towards CD70-expressing tumor cells.

As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a (β2M knockout cell if β2M protein cannot be detected at the cell surface using an antibody that specifically binds β2M protein.

In some embodiments, a disrupted gene may be described as comprising a mutated fragment relative to the wild-type counterpart. The mutated fragment may comprise a deletion, a nucleotide substitution, an addition, or a combination thereof. In other embodiments, a disrupted gene may be described as having a deletion of a fragment that is present in the wild-type counterpart. In some instances, the 5′ end of the deleted fragment may be located within the gene region targeted by a designed guide RNA such as those disclosed herein (known as on-target sequence) and the 3′ end of the deleted fragment may go beyond the targeted region. Alternatively, the 3′ end of the deleted fragment may be located within the targeted region and the 5′ end of the deleted fragment may go beyond the targeted region.

In some instances, the disrupted TRAC gene in the anti-CD70 CAR-T cells disclosed herein may comprise a deletion, for example, a deletion of a fragment in Exon 1 of the TRAC gene locus. In some examples, the disrupted TRAC gene comprises a deletion of a fragment comprising the nucleotide sequence of SEQ ID NO: 17, which is the target site of TRAC guide RNA TA-1. See sequence tables below. In some examples, the fragment of SEQ ID NO: 17 may be replaced by a nucleic acid encoding the anti-CD70 CAR. Such a disrupted TRAC gene may comprise the nucleotide sequence of SEQ ID NO: 44.

The disrupted B2M gene in the anti-CD70 CAR-T cells disclosed herein may be generated using the CRISPR/Cas technology. In some examples, a B2M gRNA provided in the sequence table below can be used. The disrupted B2M gene may comprise a nucleotide sequence of any one of SEQ ID Nos: 31-36. See Table 4 below.

Alternatively or in addition, the disrupted CD70 gene in the anti-CD70 CAR-T cells disclosed herein may be generated using the CRISPR/Cas technology. In some examples, a CD70 gRNA provided in the sequence table below can be used. The disrupted CD70 gene may comprise a nucleotide sequence of any one of SEQ ID NOs:37-42. See Table 5 below.

(iii) Exemplary Anti-CD70 CAR T Cells

In some examples, the anti-CD70 CAR T cells are CTX130 cells, which are CD70-directed T cells having disrupted TRAC gene, B2M gene, and CD70 gene. CTX130 cells can be produced via ex vivo genetic modification using CRISPR/Cas9 (Clustered Regularly Interspaced Short Palindromic Repeats/CRISPR associated protein 9) gene editing components (sgRNAs and Cas9 nuclease).

Also within the scope of the present disclosure are populations of anti-CD70 CAR T cells (e.g., a population of CTX130 cells), which comprises genetically engineered cells (e.g., CRISPR-Cas9-mediated gene edited) expressing the anti-CD70 CAR disclosed herein and disrupted TRAC, B2M, and CD70 genes; and the nucleotide sequence encoding the anti-CD70 CAR is inserted into the TRAC locus.

It should be understood that gene disruption encompasses gene modification through gene editing (e.g., using CRISPR/Cas gene editing to insert or delete one or more nucleotides). As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a (β2M knockout cell if β2M protein cannot be detected at the cell surface using an antibody that specifically binds β2M protein.

In specific instances, the anti-CD70 CAR+ T cells are CTX130 cells, which are produced using CRISPR technology to disrupt targeted genes, and adeno-associated virus (AAV) transduction to deliver the CAR construct. CRISPR-Cas9-mediated gene editing involves three guide RNAs (sgRNAs): CD70-7 sgRNA (SEQ ID NO: 2) which targets the CD70 locus, TA-1 sgRNA (SEQ ID NO: 6) which targets the TRAC locus, and B2M-1 sgRNA (SEQ ID NO: 10) which targets the β2M locus. The anti-CD70 CAR of CTX130 cells is composed of an anti-CD70 single-chain antibody fragment (scFv) specific for CD70, followed by a CD8 hinge and transmembrane domain that is fused to an intracellular co-signaling domain of 4-1BB and a CD3ζ signaling domain. As such, CTX130 is a CD70-directed T cell immunotherapy comprised of allogeneic T cells that are genetically modified ex vivo using CRISPR/Cas9 gene editing components (sgRNA and Cas9 nuclease).

In some embodiments, at least 50% of a population of CTX130 cells may not express a detectable level of β2M surface protein. For example, at least 55%, at least 60%, at least 70%, at least 75%, at least 80%, at least 85%, at least 90%, or at least 95% of the engineered T cells of a population may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

Alternatively or in addition, at least 50% of a population of CTX130 cells may not express a detectable level of TRAC surface protein. For example, at least 55%, at least 60%, at least 70%, at least 75%, at least 80%, at least 85%, at least 90%, or at least 95% of the engineered T cells of a population may not express a detectable level of TRAC surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of TRAC surface protein.

In some embodiments, at least 50% of a population of CTX130 cells may not express a detectable level of CD70 surface protein. For example, at least 55%, at least 60%, at least 70%, at least 75%, at least 80%, at least 85%, at least 90%, at least 95%, or at least 98% of the engineered T cells of a population may not express a detectable level of CD70 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, or 95%-100% of the engineered T cells of a population does not express a detectable level of CD70 surface protein.

In some embodiments, a substantial percentage of the population of CTX130 cells may comprise more than one gene edit, which results in a certain percentage of cells not expressing more than one gene and/or protein.

For example, at least 50% of a population of CTX130 cells may not express a detectable level of two surface proteins, e.g., does not express a detectable level of β2M and TRAC proteins, β2M and CD70 proteins, or TRAC and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of two surface proteins. In another example, at least 50% of a population of the CTX130 cells may not express a detectable level of all of the three target surface proteins β2M, TRAC, and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M, TRAC, and CD70 surface proteins.

In some embodiments, the population of CTX130 cells may comprise more than one gene edit (e.g., in more than one gene), which may be an edit described herein. For example, the population of CTX130 cells may comprise a disrupted TRAC gene via the CRISPR/Cas technology using guide RNA TA-1 (see also Table 2, SEQ ID NOS: 6-7). Alternatively or in addition, the population of CTX130 cells may comprise a disrupted β2M gene via CRISPR/Cas9 technology using the guide RNA of B2M-1 (see also Table 2, SEQ ID NOS: 10-11). Such CTX130 cells may comprise Indels in the β2M gene, which comprise one or more of the nucleotide sequences listed in Table 4. For example, the population of CTX130 cells may comprise a disrupted CD70 gene via the CRISPR/Cas technology using guide RNA CD70-7 (see also Table 2, SEQ ID NOS: 2-3). Further, the population of the CTX130 cells may comprise Indels in the CD70 gene, which may comprise one or more nucleotide sequences listed in Table 5.

In some embodiments, the CTX130 cells may comprise a deletion in the TRAC gene relative to unmodified T cells. For example, the CTX130 cells may comprise a deletion of the fragment AGAGCAACAGTGCTGTGGCC (SEQ ID NO: 17) in the TRAC gene, or a portion of thereof, e.g., a fragment of SEQ ID NO: 17 comprising 1, 2, 3, 4, 5, 6, 7, 8, 9, 10, 11, 12, 13, 14, 15, 16, 17, 18, or 19 consecutive base pairs. In some embodiments, the CTX130 cells include a deletion comprising the fragment of SEQ ID NO: 17 in the TRAC gene. In some embodiments, an engineered T cell comprises a deletion of SEQ ID NO: 17 in the TRAC gene relative to unmodified T cells. In some embodiments, an engineered T cell comprises a deletion comprising SEQ ID NO: 17 in the TRAC gene relative to unmodified T cells.

Further, the population of CTX130 cells may comprise cells expressing an anti-CD70 CAR such as those disclosed herein (e.g., SEQ ID NO: 46). The coding sequence of the anti-CD70 CAR may be inserted into the TRAC locus, e.g., at the region targeted by guide RNA TA-1 (see also Table 2, SEQ ID NOS: 6-7). In such instances, the amino acid sequence of the exemplary anti-CD70 CAR comprises the amino acid sequence of SEQ ID NO:46.

In some embodiments, at least 30% at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% of the CTX130 cells are CAR+ cells, which express the anti-CD70 CAR. See also WO 2019/097305A2, and WO2019/215500, the relevant disclosures of each of which are incorporated by reference for the subject matter and purpose referenced herein.

In specific examples, the anti-CD70 CAR-T cells disclosed herein (e.g., CTX130 cells) is a population of T cells having ≥30% CAR+ T cells, ≤0.4% TCR+ T cells, ≤30% B2M+ T cells, and ≤2% CD70+ T cells.

### (v) Pharmaceutical Compositions

In some aspects, the present disclosure provides pharmaceutical compositions comprising any of the populations of genetically engineered anti-CD70 CAR T cells as disclosed herein, for example, CTX130 cells, and a pharmaceutically acceptable carrier. Such pharmaceutical compositions can be used in cancer treatment in human patients, which is also disclosed herein.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions can include a pharmaceutically acceptable salt, e.g., an acid addition salt or a base addition salt. See, e.g., Berge et al., (1977) J Pharm Sci 66:1-19.

In some embodiments, the pharmaceutical composition further comprises a pharmaceutically acceptable salt. Non-limiting examples of pharmaceutically acceptable salts include acid addition salts (formed from a free amino group of a polypeptide with an inorganic acid (e.g., hydrochloric or phosphoric acids), or an organic acid such as acetic, tartaric, mandelic, or the like). In some embodiments, the salt formed with the free carboxyl groups is derived from an inorganic base (e.g., sodium, potassium, ammonium, calcium or ferric hydroxides), or an organic base such as isopropylamine, trimethylamine, 2-ethylamino ethanol, histidine, procaine, or the like).

In some embodiments, the pharmaceutical composition disclosed herein comprises a population of the genetically engineered anti-CD70 CAR-T cells (e.g., CTX130 cells) suspended in a cryopreservation solution (e.g., CryoStor® C55). The cryopreservation solution for use in the present disclosure may also comprise adenosine, dextrose, dextran-40, lactobionic acid, sucrose, mannitol, a buffer agent such as N-)2-hydroxethyl) piperazine-N′-(2-ethanesulfonic acid) (HEPES), one or more salts (e.g., calcium chloride, magnesium chloride, potassium chloride, potassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

In some instances, a pharmaceutical composition comprising a population of genetically engineered anti-CD70 CAR-T cells such as the CTX130 cells suspended in a cryopreservation solution (e.g., substantially free of serum) may be placed in storage vials.

Any of the pharmaceutical compositions disclosed herein, comprising a population of genetically engineered anti-CD70 CAR T cells as also disclosed herein (e.g., CTX130 cells), which optionally may be suspended in a cryopreservation solution as disclosed herein may be stored in an environment that does not substantially affect viability and bioactivity of the T cells for future use, e.g., under conditions commonly applied for storage of cells and tissues. In some examples, the pharmaceutical composition may be stored in the vapor phase of liquid nitrogen at ≤−135° C. No significant changes were observed with respect to appearance, cell count, viability, % CAR⁺ T cells, % TCR⁺ T cells, % B2M⁺ T cells, and % CD70⁺ T cells after the cells have been stored under such conditions for a period of time.

In some embodiments, the pharmaceutical composition disclosed herein can be a suspension for infusion, comprising the anti-CD70 CAR T cells disclosed herein such as the CTX130 cells. In some examples, the suspension may comprise about 25-85×10⁶ cells/ml (e.g., 50×10⁶ cells/ml) with ≥30% CAR+ T cells, ≤0.4% TCR+ T cells, ≤30% B2M+ T cells, and ≤2% CD70+ T cells. In some examples, the suspension may comprise about 25×10⁶ CAR+ cells/mL. In specific examples, the pharmaceutical composition may be placed in a vial, each comprising about 1.5×10⁸ CAR+ T cells such as CTX130 cells (e.g., viable cells). In other examples, the pharmaceutical composition may be placed in a vial, each comprising about 3×10⁸ CAR+ T cells such as CTX130 cells (e.g., viable cells).

### II. Preparation of Anti-CD70 CAR T Cells

Any suitable gene editing methods known in the art can be used for making the genetically engineered immune cells (e.g., T cells such as CTX130 cells) disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9). In specific examples, the genetically engineered immune cells such as CTX130 cells are produced by the CRISPR technology in combination with homologous recombination using an adeno-associated viral vector (AAV) as a donor template.

(i) CRISPR-Cas9-Mediated Gene Editing System for Genetic Engineering of

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

(a) Cas9

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 comprises a Streptococcus pyogenes-derived Cas9 nuclease protein that has been engineered to include C- and N-terminal SV40 large T antigen nuclear localization sequences (NLS). The resulting Cas9 nuclease (sNLS-spCas9-sNLS) is a 162 kDa protein that is produced by recombinant E. coli fermentation and purified by chromatography. The spCas9 amino acid sequence can be found as UniProt Accession No. Q99ZW2, which is provided herein as SEQ ID NO: 1.

Amino acid sequence of Cas9 nuclease (SEQ ID
NO: 1):
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

(b) Guide RNAs (gRNAs)

CRISPR-Cas9-mediated gene editing as described herein includes the use of a guide RNA or a gRNA. As used herein, a “gRNA” refers to a genome-targeting nucleic acid that can direct the Cas9 to a specific target sequence within a CD70 gene or a TRAC gene or a β2M gene for gene editing at the specific target sequence. A guide RNA comprises at least a spacer sequence that hybridizes to a target nucleic acid sequence within a target gene for editing, and a CRISPR repeat sequence.

An exemplary gRNA targeting a CD70 gene is provided in SEQ ID NO: 2. See also WO2019/215500, the relevant disclosures of which are incorporated by reference herein for the subject matter and purpose referenced herein. Other gRNA sequences may be designed using the CD70 gene sequence located on chromosome 19 (GRCh38: chromosome 19: 6,583,183-6,604,103; Ensembl; ENSG00000125726). In some embodiments, gRNAs targeting the CD70 genomic region and Cas9 create breaks in the CD70 genomic region resulting Indels in the CD70 gene disrupting expression of the mRNA or protein.

An exemplary gRNA targeting a TRAC gene is provided in SEQ ID NO: 6. See WO2019/097305A2, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region and Cas9 create breaks in the TRAC genomic region resulting Indels in the TRAC gene disrupting expression of the mRNA or protein.

An exemplary gRNA targeting a β2M gene is provided in SEQ ID NO: 10. See also WO 2019/097305A2, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the (β2M genomic region and RNA-guided nuclease create breaks in the β2M genomic region resulting in Indels in the β2M gene disrupting expression of the mRNA or protein.

TABLE 2
sgRNA Sequences and Target Gene Sequences.

| | | | SEQ ID |
|---|---|---|---|
| | | | NO: |
| sgRNA Sequences | | | |
| CD70 | Modified | G*C*U*UUGGUCCCAUUGGUCGCguuuuagagcuagaaau | 2 |
| sgRNA | | agcaaguuaaaauaaggcuaguccguuaucaacuugaaaaaguggcaccg | |
| (CD70-7) | | agucggugcU*U*U*U | |
| | Unmodified | GCUUUGGUCCCAUUGGUCGCguuuuagagcuagaaauagc | 3 |
| | | aaguuaaaauaaggcuaguccguuaucaacuugaaaaaguggcaccgagu | |
| | | cggugcUUUU | |
| CD70 | Modified | G*C*U*UUGGUCCCAUUGGUCGC | 4 |
| sgRNA | Unmodified | GCUUUGGUCCCAUUGGUCGC | 5 |
| spacer | | | |
| TRAC | Modified | A*G*A*GCAACAGUGCUGUGGCCguuuuagagcuagaaau | 6 |
| sgRNA | | agcaaguuaaaauaaggcuaguccguuaucaacuugaaaaaguggcaccg | |
| (TA-1) | | agucggugcU*U*U*U | |
| | Unmodified | AGAGCAACAGUGCUGUGGCCguuuuagagcuagaaauagc | 7 |
| | | aaguuaaaauaaggcuaguccguuaucaacuugaaaaaguggcaccgagu | |
| | | cggugcUUUU | |
| TRAC | Modified | A*G*A*GCAACAGUGCUGUGGCC | 8 |
| sgRNA | Unmodified | AGAGCAACAGUGCUGUGGCC | 9 |
| spacer | | | |
| β2M | Modified | G*C*U*ACUCUCUCUUUCUGGCCguuuuagagcuagaaau | 10 |
| sgRNA | | agcaaguuaaaauaaggcuaguccguuaucaacuugaaaaaguggcaccg | |
| (B2M-1) | | agucggugcU*U*U*U | |
| | Unmodified | GCUACUCUCUCUUUCUGGCCguuuuagagcuagaaauagc | 11 |
| | | aaguuaaaauaaggcuaguccguuaucaacuugaaaaaguggcaccgagu | |
| | | cggugcUUUU | |
| β2M | Modified | g*c*u*acucucucuuucuggcc | 12 |
| sgRNA | Unmodified | GCUACUCUCUCUUUCUGGCC | 13 |
| spacer | | | |

| Target Sequences (PAM) | | |
|---|---|---|
| CD70 | GCTTTGGTCCCATTGGTCGC (GGG) | 14 |
| target | | |
| sequence | | |
| with (PAM) | | |
| CD70 | GCTTTGGTCCCATTGGTCGC | 15 |
| target | | |
| sequence | | |
| TRAC | AGAGCAACAGTGCTGTGGCC (TGG) | 16 |
| target | | |
| sequence | | |
| with (PAM) | | |
| TRAC | AGAGCAACAGTGCTGTGGCC | 17 |
| target | | |
| sequence | | |
| β2M target | GCTACTCTCTCTTTCTGGCC (TGG) | 18 |
| sequence | | |
| with (PAM) | | |
| β2M target | GCTACTCTCTCTTTCTGGCC | 19 |
| sequence | | |

Exemplary sgRNA Formulas

| | | |
|---|---|---|
| sgRNA | nnnnnnnnnnnnnnnnnnnnguuuuagagcuagaaauagcaaguuaaaauaaggcuaguccg | 20 |
| sequence | uuaucaacuugaaaaaguggcaccgagucggugcuuuu | |
| sgRNA | nnnnnnnnnnnnnnnnnnnnguuuuagagcuagaaauagcaaguuaaaauaaggcuaguccg | 21 |
| sequence | uuaucaacuugaaaaaguggcaccgagucggugc | |
| sgRNA | n(17-30)guuuuagagcuagaaauagcaaguuaaaauaaggcuaguccguuaucaacuugaaa | 22 |
| sequence | aaguggcaccgagucggugcu(1-8) | |

*indicates a nucleotide with a 2′-O-methyl phosphorothioate modification,
“n” refers to the spacer sequence at the 5′ end.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence.

For example, if the CD70 target sequence is 5′-GCTTTGGTCCCATTGGTCGC-3′ (SEQ ID NO: 15), then the gRNA spacer sequence is 5′-GCUUUGGUCCCAUUGGUCGC-3′ (SEQ ID NO: 5). In another example, if the TRAC target sequence is 5′-AGAGCAACAGTGCTGTGGCC-3′ (SEQ ID NO: 17), then the gRNA spacer sequence is 5′-AGAGCAACAGUGCUGUGGCC-3′ (SEQ ID NO: 9). In yet another example, if the β2M target sequence is 5′-GCTACTCTCTCTTTCTGGCC-3′ (SEQ ID NO: 19), then the gRNA spacer sequence is 5′-GCUACUCUCUCUUUCUGGCC-3′ (SEQ ID NO: 13). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

A spacer sequence in a gRNA is a sequence (e.g., a 20 nucleotide sequence) that defines the target sequence (e.g., a DNA target sequences, such as a genomic target sequence) of a target gene of interest. An exemplary spacer sequence of a gRNA targeting a CD70 gene is provided in SEQ ID NO: 4. An exemplary spacer sequence of a gRNA targeting a TRAC gene is provided in SEQ ID NO: 8. An exemplary spacer sequence of a gRNA targeting a β2M gene is provided in SEQ ID NO: 12.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

Non-limiting examples of gRNAs that may be used as provided herein are provided in WO 2019/097305A2, and WO2019/215500, the relevant disclosures of each of the prior applications are herein incorporated by reference for the purposes and subject matter referenced herein. For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be a sgRNA, which may comprise a 20 nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence.

In some embodiments, the sgRNA comprises no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

It should be understood that more than one suitable Cas9 and more than one suitable gRNA can be used in methods described herein, for example, those known in the art or disclosed herein. In some embodiments, methods comprise a Cas9 enzyme and/or a gRNA known in the art. Examples can be found in, e.g., WO 2019/097305A2, and WO2019/215500, the relevant disclosures of each of the prior applications are herein incorporated by reference for the purposes and subject matter referenced herein.

In some embodiments, gRNAs targeting the TRAC genomic region create Indels in the TRAC gene comprising at least one nucleotide sequence selected from the sequences in Table 3. In some embodiments, the gRNA (e.g., SEQ ID NO: 6) targeting the TRAC genomic region creates Indels in the TRAC gene comprising at least one nucleotide sequence selected from the sequences in Table 3.

TABLE 3
Edited TRAC Gene Sequence.

| | Sequence (Deletions indicated | |
|---|---|---|
| Description | by dashes (-); insertions indicated by bold) | SEQ ID NO: |
| TRAC gene edit | AA---------------------GAGCAACAAATCTGACT | 23 |
| TRAC gene edit | AAGAGCAACAGTGCTGT-GCCTGGAGCAACAAATCTGACT | 24 |
| TRAC gene edit | AAGAGCAACAGTGCTG-------GAGCAACAAATCTGACT | 25 |
| TRAC gene edit | AAGAGCAACAGT------GCCTGGAGCAACAAATCTGACT | 26 |
| TRAC gene edit | AAGAGCAACAGTG---------------------CTGACT | 27 |
| TRAC gene edit | AAGAGCAACAGTGCTGTGGGCCTGGAGCAACAAATCTGACT | 28 |
| TRAC gene edit | AAGAGCAACAGTGC--TGGCCTGGAGCAACAAATCTGACT | 29 |
| TRAC gene edit | AAGAGCAACAGTGCTGTGTGCCTGGAGCAACAAATCTGACT | 30 |

In some embodiments, gRNAs targeting the β2M genomic region create Indels in the β2M gene comprising at least one nucleotide sequence selected from the sequences in Table 4. In some embodiments, the gRNA (e.g., SEQ ID NO: 10) targeting the β2M genomic region creates Indels in the β2M gene comprising at least one nucleotide sequence selected from the sequences in Table 4.

TABLE 4
Edited β2M Gene Sequence.

| | Sequence (Deletions indicated | |
|---|---|---|
| Description | by dashes (-); insertions indicated by bold) | SEQ ID NO: |
| β2M gene-edit | CGTGGCCTTAGCTGTGCTCGCGCTACTCTCTCTTTCT- | 31 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTTC-- | 32 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTT----- | 33 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTCTGGATAGCCTGGAGGC | 34 |
| | TATCCAGCGTGAGTCTCTCCTACCCTCCCGCT | |
| β2M gene-edit | CGTGGCCTTAGCTGTGCTCGC------------------------- | 35 |
| | GCTATCCAGCGTGAGTCTCTCCTACCCTCCCGCT | |
| β2M gene-edit | CGTGGCCTTAGCTGTGCTCGCGCTACTCTCTCTTTCTGTGGCCTGGAGGCTA | 36 |
| | TCCAGCGTGAGTCTCTCCTACCCTCCCGCT | |

In some embodiments, gRNAs targeting the CD70 genomic region create Indels in the CD70 gene comprising at least one nucleotide sequence selected from the sequences in Table 5. In some embodiments, the gRNA (e.g., SEQ ID NO: 2) targeting the CD70 genomic region creates Indels in the CD70 gene comprising at least one nucleotide sequence selected from the sequences in Table 5.

TABLE 5
Edited CD70 Gene Sequence.

| | Sequence (Deletions indicated | |
|---|---|---|
| Description | by dashes (-); insertions indicated by bold) | SEQ ID NO: |
| CD70 gene-edit | CACACCACGAGGCAGATCACCAAGCCCGCG -- | 37 |
| | CAATGGGACCAAAGCAGCCCGCAGGACG | |
| CD70 gene-edit | CACACCACGAGGCAGATCACCAAGCCCGCGAACCAATGGGACCAAAGCAGCC | 38 |
| | CGCAGGACG | |
| CD70 gene-edit | CACACCACGAGGCAGATC------------ | 39 |
| | ACCAATGGGACCAAAGCAGCCCGCAGGACG | |
| CD70 gene-edit | CACACCACGAGGCAGATCACCAAGCCCGCG- | 40 |
| | CCAATGGGACCAAAGCAGCCCGCAGGACG | |
| CD70 gene-edit | CACACCACGAGGCAGATCACCAAGCCCGC- | 41 |
| | ACCAATGGGACCAAAGCAGCCCGCAGGACG | |
| CD70 gene-edit | CACACCACGAGGCAGATCACCA------------------------- | 42 |
| | AGCCCGCAGGACG | |

(iii) AAV Vectors for Delivery of CAR Constructs to T Cells

A nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids, which confer AAV serotype, which determines which target organs the capsids primarily binds and thus what cells the AAV most efficiently infects. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding a CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions can be used for this purpose, e.g., those disclosed herein.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions, and inserting a CAR coding segment into the TRAC gene.

A donor template as disclosed herein can contain a coding sequence for a CAR. In some examples, the CAR-coding sequence may be flanked by two regions of homology to allow for efficient HDR at a genomic location of interest, for example, at a TRAC gene using CRISPR-Cas9 gene editing technology. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous promoter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

### III. Treatment of Hematopoietic Cell Malignancies

In some aspects, provided herein are methods for treating a human patient having a hematopoietic cell malignancy (e.g., a T cell or B cell malignancy, or a myeloid cell malignancy) using a population of any of the anti-CD70 CAR T cells such as the CTX130 cells as disclosed herein. The allogeneic anti-CD70 CAR T cell therapy may comprise two stages of treatment (i) a conditioning regimen (lymphodepleting treatment), which comprises giving one or more doses of one or more lymphodepleting agents to a suitable human patient, and (ii) a treatment regimen (anti-CD70 CAR T cell therapy), which comprises administration of the population of anti-CD70 CAR T cells such as the CTX130 cells as disclosed herein to the human patient. When applicable, multiple doses of the anti-CD70 CAR T cells may be given to the human patient and a lymphodepletion treatment can be applied to the human patient prior to each dose of the anti-CD70 CAR T cells.

### (i) Patient Population

A human patient may be any human subject for whom diagnosis, treatment, or therapy is desired. A human patient may be of any age. In some embodiments, the human patient is an adult (e.g., a person who is at least 18 years old). In some embodiments, the human patient is a child. In some embodiments, the human patient has a body weight≥60 kg.

A human patient to be treated by the methods described herein can be a human patient having, suspected of having, or a risk for having a hematopoietic cell malignancy (e.g., comprising CD70+ disease cells). In some examples, the human patient has, is suspected of having, or is at risk for a T cell malignancy. In some examples, the human patient has, is suspected of having, or is at risk for a B cell malignancy. In some examples, the human patient has, is suspected of having, or is at risk for a myeloid cell malignancy. A subject suspected of having a hematopoietic cell malignancy might show one or more symptoms of the hematopoietic cell malignancy, e.g., unexplained weight loss, fatigue, night sweats, shortness of breath, or swollen glands. A subject at risk for a hematopoietic cell malignancy can be a subject having one or more of the risk factors for a hematopoietic cell malignancy, e.g., a weakened immune system, age, male, or infection (e.g., Epstein-Barr virus infection). A human patient who needs the anti-CD70 CAR T cell (e.g., CTX130 cell) treatment may be identified by routine medical examination, e.g., physical examination, laboratory tests, biopsy (e.g., bone marrow biopsy and/or lymph node biopsy), magnetic resonance imaging (MRI) scans, or ultrasound exams.

In some embodiments, the human patient has a T cell malignancy, e.g., a relapsed or refractory T cell malignancy. Such a human patient may carry CD70+ disease T cells. Examples include, but are not limited to, cutaneous T-cell lymphoma (CTCL), peripheral T-cell lymphoma (PTCL), and T cell leukemia. In some instances, the T cell malignancy can be CTCL, which may include mycosis fungoides (MF), for example, stage IIb or higher, including transformed large cell lymphoma, or Sezary Syndrome (SS).

In some instances, the T cell malignancy is PTCL. Examples include, but are not limited to, angioimmunoblastic T cell lymphoma (AITL), anaplastic large cell lymphoma (ALCL), which may be Alk positive or Alk negative, adult T cell leukemia or lymphoma (ATLL), which may exclude the smoldering subtype (non-smoldering ATLL); and peripheral T-cell lymphoma not otherwise (PTCL-NOS).

In some embodiments, the human patient may have a B cell malignancy, for example, a relapsed or refractory B cell malignancy. Such a human patient may carry CD70+ disease B cells. In some examples, the human patient has diffused large B cell lymphoma (DLBCL). Such a human patient may have failed a prior anti-CD19 CAR-T cell therapy. In other examples, the human patient has mantle cell lymphoma (MCL), which is an aggressive type of B-cell non-Hodgkin lymphoma (NHL) associated with poor prognosis.

In yet other embodiments, the human patient may have a myeloid cell malignancy, for example, a relapsed or refractory myeloid cell malignancy. In some examples, the human patient has acute myeloid leukemia (AML, also referred to as acute myelogenous leukemia).

In some embodiments, the human patient has a CD70+ leukemia. In some embodiments, the human patient has a CD70+ T cell leukemia. In some embodiments, the human patient has a CD70+ lymphoma. In some embodiments, the human patient has a CD70+ T cell lymphoma.

In some embodiments, the human patient to be treated by the methods described herein can be a human patient having a tumor comprising CD70-expressing tumor cells (CD70-expressing tumor), which may be identified by any method known in the art. For example, a CD70-expressing tumor may be identified by immunohistochemistry (IHC) in tissue collected by excisional or core biopsy of a representative tumor. In another example, a CD70-expressing tumor may be identified by flow cytometry in tumor cells defined by immunophenotyping collected in the peripheral blood or bone marrow. In specific examples, the human patient to be treated by the method disclosed herein may have a tumor comprising at least 10% CD70⁺ tumor cells in the total cancer cells in a biological sample (e.g., a tissue sample such as a lymph node sample, a blood sample or a bone marrow sample).

Any of the methods disclosed herein may further comprise a step of identifying a human patient suitable for the allogeneic anti-CD70 CAR T therapy based on presence and/or level of CD70+ tumor cells in the patient. The identifying step can be performed by determining presence and/or level of CD70+ tumor cells in a biopsy sample obtained from a candidate patient via, e.g., IHC. Alternatively, the identifying step can be performed by determining presence and/or level of CD70+ tumor cells in a blood sample or a bone marrow sample obtained from the candidate patient via, e.g., flow cytometry.

A human patient to be treated by methods described herein may be a human patient that has relapsed following a treatment and/or that has been become resistant to a treatment and/or that has been non-responsive to a treatment. Non-limiting examples include a patient that has: (a) relapsed or refractory hematopoietic cell malignancy (e.g., T cell or B cell malignancies, or myeloid cell malignancy), (b) SS or mycosis fungoides (MF)≥Stage IIB, who may be in need of transplant, (c) diffuse large B cell lymphoma (DLBCL), who may be non-responsive to anti-CD19 CAR T cell therapy, (d) PTCL, ATLL (e.g., leukemic ATLL, lymphomatous ATLL), or AITL and has failed a first line systemic therapy, (e) ALCL and has failed a combined therapy comprising breutuximab vedotin, (f) ALK+ ALCL and has failed two prior lines of therapy (for example, one of such may comprise brentuximab vedotin), (g) ALK− ALCL and has failed one prior line of therapy, or (h) MF or SS and has failed one or more (e.g., at least two) prior systemic therapies, which, in some instances, may comprise a prior mogamulizumab therapy.

A human patient to be treated by methods described herein may be a human patient that has had recent prior treatment or a patient that is free of prior treatment. For example, a human patient to be treated as described herein may be free of mogamulizumab treatment at least three months prior to the first dose of the population of genetically modified T cells.

Any of the human patients treated using a method disclosed herein may receive subsequent treatment. For example, the human patient is subject to an anti-cytokine therapy. In another example, the human patient is subject to autologous or allogeneic hematopoietic stem cell transplantation after treatment with the population of genetically engineered T cells.

A human patient may be screened to determine whether the patient is eligible to undergo a conditioning regimen (lymphodepleting treatment) and/or a treatment regimen (anti-CD70 CAR T cell therapy). For example, a human patient who is eligible for lymphodepletion treatment does not show one or more of the following features: (a) change in performance status to Eastern Cooperative Oncology Group (ECOG)>1, (b) significant worsening of clinical status (e.g., significant worsening of clinical status that may increase the potential risk of AEs associated with the conditioning regimen and/or the treatment regimen), (c) requirement for supplemental oxygen to maintain a saturation level of greater than 92%, (d) uncontrolled cardiac arrhythmia, (e) hypotension requiring vasopressor support, (f) active infection, and (g) any acute neurological toxicity (e.g., ≥2 acute neurological toxicity).

In another example, a human patient who is eligible for a treatment regimen does not show one or more of the following features: (a) change in performance status to Eastern Cooperative Oncology Group (ECOG)>1, (b) active uncontrolled infection, (c) significant worsening of clinical status (e.g., significant worsening of clinical status that may increase the potential risk of AEs associated with allogenic CAR T cell infusion), and (d) any acute neurological toxicity (e.g., ≥2 acute neurological toxicity).

Significant worsening of clinical status that may increase the potential risk of AEs associated with the conditioning regimen and/or the treatment regimen may include, but is not limited to, clinically significant worsening of cytopenia, clinically significant increase of transaminase levels (e.g., >3×ULN), clinically significant increase of total bilirubin (e.g., >2×ULN), and clinically significant increase in serum creatinine.

A human patient may be screened and excluded from the conditioning regimen and/or treatment regimen based on such screening results. For example, a human patient may be excluded from a conditioning regimen and/or a treatment regimen if the patient meets any of the following exclusion criteria: (a) prior allogeneic stem cell transplant (SCT), (b) less than 60 days from autologous SCT at time of screening and with unresolved serious complications, (c) prior treatment with any anti-CD70 targeting agents, (d) prior treatment with any CAR T cells or any other modified T or natural killer (NK) cells except autologous CD19 CAR T cells, and the patient has DLBCL, (e) known contraindication to any lymphodepletion treatment or any of the excipients of any treatment regimen, (f) T cell or B cell lymphomas with a present or past malignant effusion that is or was symptomatic, (g) clinical signs of hemophagocytic lymphohistiocytosis (HLH), (h) detectable malignant cells from cerebrospinal fluid (CSF) or magnetic resonance imaging (MRI) indicating brain metastases, (i) history or presence of clinically relevant CNS pathology, (j) unstable angina, arrhythmia, or myocardial infarction within 6 months prior to screening, (k) previous or concurrent malignancy, except those treated with a curative approach who have been in remission for >12 months without requiring systemic therapy (in some instances, basal cell or squamous cell skin carcinoma, adequately resected and in situ carcinoma of cervix, or a previous malignancy that was completely resected and has been in remission for greater than 3 years may be allowed), and (l) uncontrolled, acute life-threatening bacterial, viral, or fungal infection.

A human patient subjected to lymphodepletion treatment may be screened for eligibility to receive one or more doses of the anti-CD70 CAR T cells disclosed herein such as the CTX130 cells. For example, a human patient subjected to lymphodepletion treatment that is eligible for an anti-CD70 CAR T cell treatment does not show one or more of the following features: (a) change in performance status to Eastern Cooperative Oncology Group (ECOG)>1, (b) active uncontrolled infection, (c) significant worsening of clinical status (e.g., significant worsening of clinical status that may increase the potential risk of AEs associated with allogenic CAR T cell infusion), and (d) any acute neurological toxicity (e.g., ≥2 acute neurological toxicity).

Following each dosing of anti-CD70 CAR T cells, a human patient may be monitored for acute toxicities such as cytokine release syndrome (CRS), tumor lysis syndrome (TLS), neurotoxicity (e.g., immune effector cell-associated neurotoxicity syndrome or ICANS), and graft versus host disease (GvHD). In addition, one or more of the following adverse effects may be monitored: hypotension, renal insufficiency (which may be caused, e.g., by suppression of renal tubular-like epithelium cells), hemophagocytic lymphohistiocytosis (HLH), prolonged cytopenias, and/or suppression of osteoblasts. After each dose of anti-CD70 CAR T cells, a human patient may be monitored for at least 28 days for development of toxicity.

When a human patient exhibits one or more symptoms of acute toxicity, the human patient may be subjected to toxicity management. Treatments for patients exhibiting one or more symptoms of acute toxicity are known in the art. For example, a human patient exhibiting a symptom of CRS (e.g., cardiac, respiratory, and/or neurological abnormalities) may be administered an anti-cytokine therapy. In addition, a human patient that does not exhibit a symptom of CRS may be administered an anti-cytokine therapy to promote proliferation of anti-CD70 CAR T cells.

Alternatively, or in addition to, when a human patient exhibits one or more symptoms of acute toxicity, treatment of the human patient may be terminated. Patient treatment may also be terminated if the patient exhibits one or more signs of an adverse event (AE), e.g., the patient has an abnormal laboratory finding and/or the patient shows signs of disease progression.

### (ii) Conditioning Regimen (Lymphodepleting Therapy)

Any human patients suitable for the treatment methods disclosed herein may receive a lymphodepleting therapy to reduce or deplete the endogenous lymphocyte of the subject.

Lymphodepletion refers to the destruction of endogenous lymphocytes and/or T cells, which is commonly used prior to immunotransplantation and immunotherapy. Lymphodepletion can be achieved by irradiation and/or chemotherapy. A “lymphodepleting agent” can be any molecule capable of reducing, depleting, or eliminating endogenous lymphocytes and/or T cells when administered to a subject. In some embodiments, the lymphodepleting agents are administered in an amount effective in reducing the number of lymphocytes by at least 10%, 20%, 30%, 40%, 50%, 60%, 70%, 80%, 90%, 95%, 96%, 96%, 97%, 98%, or at least 99% as compared to the number of lymphocytes prior to administration of the agents. In some embodiments, the lymphodepleting agents are administered in an amount effective in reducing the number of lymphocytes such that the number of lymphocytes in the subject is below the limits of detection. In some embodiments, the subject is administered at least one (e.g., 2, 3, 4, 5 or more) lymphodepleting agents.

In some embodiments, the lymphodepleting agents are cytotoxic agents that specifically kill lymphocytes. Examples of lymphodepleting agents include, without limitation, fludarabine, cyclophosphamide, bendamustin, 5-fluorouracil, gemcitabine, methotrexate, dacarbazine, melphalan, doxorubicin, vinblastine, cisplatin, oxaliplatin, paclitaxel, docetaxel, irinotecan, etopside phosphate, mitoxantrone, cladribine, denileukin diftitox, or DAB-IL2. In some instances, the lymphodepleting agent may be accompanied with low-dose irradiation. The lymphodepletion effect of the conditioning regimen can be monitored via routine practice.

In some embodiments, the method described herein involves a conditioning regimen that comprises one or more lymphodepleting agents, for example, fludarabine and cyclophosphamide. A human patient to be treated by the method described herein may receive multiple doses of the one or more lymphodepleting agents for a suitable period (e.g., 1-5 days) in the conditioning stage. The patient may receive one or more of the lymphodepleting agents once per day during the lymphodepleting period. In one example, the human patient receives fludarabine at about 20-50 mg/m² (e.g., 20 mg/m² or 30 mg/m²) per day for 2-4 days (e.g., 3 days) and cyclophosphamide at about 300-600 mg/m² (e.g., 500 mg/m²) per day for 2-4 days (e.g., 3 days). In another example, the human patient receives fludarabine at about 20-30 mg/m² (e.g., 25 mg/m²) per day for 2-4 days (e.g., 3 days) and cyclophosphamide at about 300-500 mg/m² (e.g., 300 mg/m² or 400 mg/m²) per day for 2-4 days (e.g., 3 days). If needed, the dose of cyclophosphamide may be increased, for example, to up to 1,000 mg/m².

The human patient may then be administered any of the anti-CD70 CAR T cells such as CTX130 cells within a suitable period after the lymphodepleting therapy as disclosed herein. For example, a human patient may be subject to one or more lymphodepleting agent about 2-7 days (e.g., for example, 2, 3, 4, 5, 6, 7 days) before administration of the anti-CD70 CAR+ T cells (e.g., CTX130 cells).

Since the allogeneic anti-CD70 CAR-T cells such as CTX130 cells can be prepared in advance, the lymphodepleting therapy as disclosed herein may be applied to a human patient having a T cell or B cell malignancy within a short time window (e.g., within 2 weeks) after the human patient is identified as suitable for the allogeneic anti-CD70 CAR-T cell therapy disclosed herein.

Methods described herein encompass redosing a human patient with anti-CD70 CAR+ T cells. In such instances, the human patient is subjected to lymphodepletion treatment prior to redosing. For example, a human patient may be subject to a first lymphodepletion treatment and a first dose of CTX130 followed by a second lymphodepletion treatment and a second dose of CTX130. In another example, a human patient may be subject to a first lymphodepletion treatment and a first dose of CTX130, a second lymphodepletion treatment and a second dose of CTX130, and a third lymphodepletion treatment and a third dose of CTX130.

Prior to any of the lymphodepletion steps (e.g., prior to the initial lymphodepletion step or prior to any follow-on lymphodepletion step in association with a re-dosing of the anti-CD70 CAR T cells such as CTX130 cells), a human patient may be screened for one or more features to determine whether the patient is eligible for lymphodepletion treatment. For example, prior to lymphodepletion, a human patient eligible for lymphodepletion treatment does not show one or more of the following features: (a) change in performance status to Eastern Cooperative Oncology Group (ECOG)>1, (b) significant worsening of clinical status (e.g., significant worsening of clinical status that may increase the potential risk of AEs associated with lymphodepletion treatment), (c) requirement for supplemental oxygen to maintain a saturation level of greater than 92%, (d) uncontrolled cardiac arrhythmia, (e) hypotension requiring vasopressor support, (f) active infection, and (g) any acute neurological toxicity (e.g., ≥2 acute neurological toxicity). In some examples, significant worsening of clinical status that may increase potential risk of adverse events associated with lymphodepletion treatment includes, but is not limited to, clinically significant worsening of any cytopenia, clinically significant increase of transaminase levels (e.g., >3×ULN), clinically significant increase of total bilirubin (e.g., >2×ULN), and/or clinically significant increase in serum creatinine.

Following lymphodepletion, a human patient may be screened for one or more features to determine whether the patient is eligible for treatment with anti-CD70 CAR T cells. For example, prior to anti-CD70 CAR T cell treatment and after lymphodepletion treatment, a human patient eligible for anti-CD70 CAR T cells treatment does not show one or more of the following features: (a) change in performance status to Eastern Cooperative Oncology Group (ECOG)>1, (b) active uncontrolled infection, (c) significant worsening of clinical status (e.g., significant worsening of clinical status that may increase the potential risk of AEs associated with allogenic CAR T cell infusion), and (d) any acute neurological toxicity (e.g., ≥2 acute neurological toxicity).

(iii) Administration of Anti-CD70 CAR T Cells

Aspects of the present disclosure provide methods of treating a T cell or B cell malignancy comprising subjecting a human patient to lymphodepletion treatment and administering to the human patient a dose of a population of genetically engineered T cells described herein (e.g., CTX130 cells).

Administering anti-CD70 CAR T cells may include placement (e.g., transplantation) of a genetically engineered T cell population into a human patient by a method or route that results in at least partial localization of the genetically engineered T cell population at a desired site, such as a tumor site, such that a desired effect(s) can be produced. The genetically engineered T cell population can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects described herein, an effective amount of the genetically engineered T cell population can be administered via a systemic route of administration, such as an intraperitoneal or intravenous route.

In some embodiments, the genetically engineered T cell population is administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

An effective amount refers to the amount of a genetically engineered T cell population needed to prevent or alleviate at least one or more signs or symptoms of a medical condition (e.g., a T cell or B cell malignancy), and relates to a sufficient amount of a genetically engineered T cell population to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

An effective amount of a genetically engineered T cell population may comprise about 1×10⁷ CAR+ cells to about 1×10⁹ CAR+ cells, e.g., about 3×10⁷ cells to about 1×10⁹ cells that express a CAR that binds CD70.

An effective amount of a genetically engineered T cell population may comprise about 3.0×10⁷ cells to about 9×10⁸ cells that express an anti-CD70 CAR (CAR⁺ cells), for example, CAR⁺ CTX130 cells. In some embodiments, an effective amount of a genetically engineered T cell population may comprise at least 3.0×10⁸ CAR⁺ CTX130 cells, at least 4×10⁸ CAR⁺ CTX130 cells, at least 4.5×10⁸ CAR⁺ CTX130 cells, at least 5×10⁸ CAR⁺ CTX130 cells, at least 5.5×10⁸ CAR⁺ CTX130 cells, at least 6×10⁸ CAR⁺ CTX130 cells, at least 6.5×10⁸ CAR⁺ CTX130 cells, at least 7×10⁸ CAR⁺ CTX130 cells, at least 7.5×10⁸ CAR⁺ CTX130 cells, at least 8×10⁸ CAR⁺ CTX130 cells, at least 8.5×10⁸ CAR⁺ CTX130 cells, or at least 9×10⁸ CAR⁺ CTX130 cells. In some examples, the amount of the CAR⁺ CTX130 cells may not exceed 1×10⁹ cells.

In some embodiments, an effective amount of the genetically engineered T cell population as disclosed herein (e.g., the CTX130 cells) may range from about 3.0×10⁷ to about 3×10⁸ CAR⁺ T cells, for example, about 1×10⁷ to about 1×10⁸ CAR⁺ T cells or about 1×10⁸ to about 3×10⁸ CAR⁺ T cells. In some embodiments, an effective amount of the genetically engineered T cell population as disclosed herein (e.g., the CTX130 cells) may range from about 1.5×10⁸ to about 3×10⁸ CAR⁺ T cells.

In some embodiments, an effective amount of the genetically engineered T cell population as disclosed herein (e.g., the CTX130 cells) may range from about 3.0×10⁸ to about 9×10⁸ CAR⁺ T cells, for example, about 3.5×10⁸ to about 6×10⁸ CAR⁺ T cells or about 3.5×10⁸ to about 4.5×10⁸ CAR⁺ T cells. In some embodiments, an effective amount of the genetically engineered T cell population as disclosed herein (e.g., the CTX130 cells) may range from about 4.5×10⁸ to about 9×10⁸ CAR⁺ T cells. In some embodiments, an effective amount of the genetically engineered T cell population as disclosed herein (e.g., the CTX130 cells) may range from about 4.5×10⁸ to about 6×10⁸ CAR⁺ T cells. In some embodiments, an effective amount of the genetically engineered T cell population as disclosed herein (e.g., the CTX130 cells) may range from about 6×10⁸ to about 9×10⁸ CAR⁺ T cells. In some embodiments, an effective amount of the genetically engineered T cell population as disclosed herein (e.g., the CTX130 cells) may range from about 7.5×10⁸ to about 9×10⁸ CAR⁺ T cells.

In specific examples, an effective amount of the genetically engineered T cell population as disclosed herein (e.g., the CTX130 cells) may comprise about 3.0×10⁸ CAR⁺ T cells. For example, an effective amount of the genetically engineered T cell population as disclosed herein (e.g., the CTX130 cells) may comprise about 4.5×10⁸ CAR⁺ T cells. In other examples, an effective amount of the genetically engineered T cell population as disclosed herein (e.g., the CTX130 cells) may comprise about 6×10⁸ CAR⁺ T cells. In some examples, an effective amount of the genetically engineered T cell population as disclosed herein (e.g., the CTX130 cells) may comprise about 7.5×10⁸ CAR⁺ T cells. In yet other examples, an effective amount of the genetically engineered T cell population as disclosed herein (e.g., the CTX130 cells) may comprise about 9×10⁸ CAR⁺ T cells.

In some embodiments, an effective amount of the genetically engineered T cell population as disclosed herein (e.g., the CTX130 cells) may range from about 3×10⁸ to about 9×10⁸ CAR⁺ T cells. In some embodiments, an effective amount of the genetically engineered T cell population as disclosed herein (e.g., the CTX130 cells) may range from about 3×10⁸ to about 7.5×10⁸ CAR⁺ T cells. In some embodiments, an effective amount of the genetically engineered T cell population as disclosed herein (e.g., the CTX130 cells) may range from about 3×10⁸ to about 6×10⁸ CAR⁺ T cells. In some embodiments, an effective amount of the genetically engineered T cell population as disclosed herein (e.g., the CTX130 cells) may range from about 3×10⁸ to about 4.5×10⁸ CAR⁺ T cells.

In some embodiments, an effective amount of a genetically engineered T cell population may comprise a dose of the genetically engineered T cell population, e.g., a dose comprising about 3.0×10⁸ CAR⁺ CTX130 cells to about 9×10⁸ CAR⁺ CTX130 cells, e.g., any dose or range of doses disclosed herein. In some examples, the effective amount is 4.5×10⁶ CAR⁺ CTX130 cells. In some examples, the effective amount is 6×10⁸ CAR⁺ CTX130 cells. In some examples, the effective amount is 7.5×10⁸ CAR⁺ CTX130 cells. In some examples, the effective amount is 9×10⁸ CAR⁺ CTX130 cells.

In some examples, a patient having CTCL, for example mycosis fungoides (MF) with large cell transformation, may be given a suitable dose of CTX130 cells, for example, about 3×10⁷ to about 6×10⁸ CAR⁺ CTX130 cells. Such an MF patient may be administered about 3×10⁷ CAR⁺ CTX130 cells. Alternatively, the MF patient may be administered about 1×10⁸ CAR⁺ CTX130 cells. In another example, the MF patient may be administered about 3×10⁸ CAR⁺ CTX130 cells. In another example, the MF patient may be administered about 4.5×10⁸ CAR⁺ CTX130 cells. In another example, the MF patient may be administered about 6×10⁸ CAR⁺ CTX130 cells. In another example, the MF patient may be administered about 7.5×10⁸ CAR⁺ CTX130 cells. In another example, the MF patient may be administered about 9×10⁸ CAR⁺ CTX130 cells.

In some examples, a patient having CTCL, for example mycosis fungoides (MF) with large cell transformation, may be given a suitable dose of CTX130 cells, for example, about 9×10⁹ to about 1×10⁹ CAR⁺ CTX130 cells. Such an MF patient may be administered about 9×10⁹ CAR⁺ CTX130 cells. Alternatively, the MF patient may be administered about 1×10⁹ CAR⁺ CTX130 cells.

In some embodiments, a suitable dose of CTX130 cells administered from one or more vials of the pharmaceutical composition, each vial comprising about 1.5×10⁸ CAR+ CTX130 cells. In some embodiments, a suitable dose of CTX130 cells is administered from one or more vials of the pharmaceutical composition, each vial comprising about 3×10⁸ CAR+ CTX130 cells. In some embodiments, a suitable dose of CTX130 cells is administered to a subject in one or more folds of 1.5×10⁸ CAR+ CTX130 cells, e.g., 1-fold, 2-fold, 3-fold, 4-fold, 5-fold, or 6-fold of 1.5×10⁸ CAR+ CTX130 cells. In some embodiments, a suitable dose of CTX130 cells is administered from one or more full or partial vials of the pharmaceutical composition.

The efficacy of anti-CD70 CAR T cell therapy described herein can be determined by the skilled clinician. An anti-CD70 CAR T cell therapy is considered “effective”, if any one or all of the signs or symptoms of, as but one example, levels of CD70 are altered in a beneficial manner (e.g., decreased by at least 10%), or other clinically accepted symptoms or markers of a T cell or B cell malignancy are improved or ameliorated. Efficacy can also be measured by failure of a subject to worsen as assessed by hospitalization or need for medical interventions (e.g., progression of the T cell or B cell malignancy is halted or at least slowed). Methods of measuring these indicators are known to those of skill in the art and/or described herein.

Treatment includes any treatment of a T cell or B cell malignancy in a human patient and includes: (1) inhibiting the disease, e.g., arresting, or slowing the progression of symptoms; or (2) relieving the disease, e.g., causing regression of symptoms; and (3) preventing or reducing the likelihood of the development of symptoms.

Treatment methods described herein encompass repeating lymphodepletion and redosing of anti-CD70 CAR T cells. Prior to each redosing of anti-CD70 CAR T cells, the patient is subjected to another lymphodepletion treatment. The doses of anti-CD70 CAR T cells may be the same for the first, second, and third doses. For example, each of the first, second, and third doses can be 1×10⁷ CAR⁺ cells, 3×10⁷ CAR⁺ cells, 1×10⁸ CAR⁺ cells, 1.5×10⁸ CAR⁺ cells, 3×10⁸ CAR⁺ cells, 4.5×10⁸ CAR⁺ cells, 6×10⁸ CAR⁺ cells, 7.5×10⁸ CAR⁺ cells, or 9×10⁸ CAR⁺ cells. In other instances, the doses of anti-CD70 CAR T cells may increase in number of CAR+ cells as the number of doses increases. For example, the first dose is 1×10⁷ CAR+ cells, the second dose is 1×10⁸ CAR+ cells, and the third dose is 1×10⁹ CAR+ cells. Alternatively, the first dose of CAR+ cells is lower than the second and/or third dose of CAR+ cells, e.g., the first dose is 1×10⁷ CAR+ cells and the second and the third doses are 1×10⁹ CAR+ cells. In some examples, the dose of anti-CD70 CAR T cells may increase by 1.5×10⁸ CAR+ cells for each subsequent dose.

Patients may be assessed for re-dosing following each administration of anti-CD70 CAR T cells. For example, following a first dose of anti-CD70 CAR T cells, a human patient may be eligible for receiving a second dose of anti-CD70 CAR T cells if the patient does not show one or more of the following: (a) dose-limiting toxicity (DLT), (b) grade 4 CRS that does not resolve to grade 2 within 72 hours, (c) grade>1 GvHD, (d) grade≥3 neurotoxicity, (e) active infection, (f) hemodynamically unstable, and (g) organ dysfunction. In another example, following a second dose of anti-CD70 CAR T cells, a human patient may be eligible for receiving a third dose of CTX130 if that patient does not show one or more of the following: (a) dose-limiting toxicity (DLT), (b) grade 4 CRS that does not resolve to grade 2 within 72 hours, (c) grade>1 GvHD, (d) grade≥3 neurotoxicity, (e) active infection, (f) hemodynamically unstable, and (g) organ dysfunction.

In some embodiments, a human patient as disclosed herein may be given multiple doses of the anti-CD70 CAR T cells (e.g., the CTX130 cells as disclosed herein), i.e., re-dosing. The human patient may be given up to three doses in total (i.e., re-dosing for no more than 2 times). The interval between two consecutive doses may be about 8 weeks to about 2 years. In some examples, a human patient may be re-dosed if the patient achieved a partial response (PR) or complete response (CR) after a first dose (or a second dose) and subsequently progressed within 2 years of last dose. In other examples, a human patient may be re-dosed when the patient achieved PR (but not CR) or stable disease (SD) after the most recent dose.

In some instances, re-dosing of anti-CD70 CAR T cells may take place up to 12 weeks after the first dose of anti-CD70 CAR T cells. A human patient may be re-dosed for up to two times at 12 weeks. When a patient is administered two doses, the second dose may be administered 3-6 weeks or 9-12 weeks after the first dose. When a patient is administered three doses, the third dose may be administered 9-12 weeks after the first dose, and the second dose may be administered 3-6 weeks after the first dose.

Following each dosing of anti-CD70 CAR T cells, a human patient may be monitored for acute toxicities such as cytokine release syndrome (CRS), tumor lysis syndrome (TLS), neurotoxicity, graft versus host disease (GvHD), and/or on target off-tumor toxicities (e.g., due to the activity of the anti-CD70 CAR T cells against activated T lymphocytes, B lymphocytes, dendritic cells, osteoblasts, and/or renal tubular-like epithelium) and/or uncontrolled T cell proliferation. In addition, one or more of the following adverse effects may be monitored: hypotension, renal insufficiency (which may be caused, e.g., by suppression of renal tubular-like epithelium cells), hemophagocytic lymphohistiocytosis (HLH), prolonged cytopenias, and/or suppression of osteoblasts. After each dose of anti-CD70 CAR T cells, a human patient may be monitored for at least 28 days for development of toxicity. If development of toxicity is observed, the human patient may be subjected to toxicity management. Treatments for patients exhibiting one or more symptoms of acute toxicity are known in the art. For example, a human patient exhibiting a symptom of CRS (e.g., cardiac, respiratory, and/or neurological abnormalities) may be administered an anti-cytokine therapy. In addition, a human patient that does not exhibit a symptom of CRS may be administered an anti-cytokine therapy to promote proliferation of anti-CD70 CAR T cells.

Anti-CD70 CAR T cell treatment methods described herein may be used on a human patient that has undergone a prior anti-cancer therapy such as a prior anti-CD19 CAR T cell therapy, a prior first line systemic therapy, a prior combined therapy, or a prior mogamulizumab therapy.

Anti-CD70 CAR T cells treatment methods described herein may also be used in combination therapies. For example, anti-CD70 CAR T cells treatment methods described herein may be co-used with other therapeutic agents, for treating a T cell or a B cell malignancy, or for enhancing efficacy of the genetically engineered T cell population and/or reducing side effects of the genetically engineered T cell population.

### IV. Kit for Treating Hematopoietic Cell Malignancies

The present disclosure also provides kits for use of a population of anti-CD70 CAR T cells such as CTX130 cells as described herein in methods for treating a hematopoietic cell malignancy, e.g., a T cell malignancy, a B cell malignancy, or a myeloid cell malignancy. Such kits may include one or more containers comprising a first pharmaceutical composition that comprises one or more lymphodepleting agents, and a second pharmaceutical composition that comprises any nucleic acid or population of genetically engineered T cells (e.g., those described herein), and a pharmaceutically acceptable carrier.

In some embodiments, the kit can comprise instructions for use in any of the methods described herein. The included instructions can comprise a description of administration of the first and/or second pharmaceutical compositions to a subject to achieve the intended activity in a human patient. The kit may further comprise a description of selecting a human patient suitable for treatment based on identifying whether the human patient is in need of the treatment. In some embodiments, the instructions comprise a description of administering the first and second pharmaceutical compositions to a human patient who is in need of the treatment.

The instructions relating to the use of a population of anti-CD70 CAR T cells such as CTX130 cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the population of genetically engineered T cells is used for treating, delaying the onset, and/or alleviating a hematopoietic cell (e.g., T cell, B cell, or myeloid cell) malignancy in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of the anti-CD70 CAR-T cells such as the CTX130 cells as disclosed herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any way as limiting their scope.

### Example 1: Generation of T Cells with Multiple Gene Knockouts

This example describes the use of CRISPR/Cas9 gene editing technology to produce human T cells that lack expression of two or three genes simultaneously. Specifically, the T cell receptor (TCR) gene (gene edited in the TCR Alpha Constant (TRAC) region), the β2-microglobulin (β2M) gene, and the Cluster of Differentiation 70 (CD70) gene were edited by CRISPR/Cas9 gene editing to produce T cells deficient in two or more of the listed genes. The following abbreviations are used in for brevity and clarity:

2×KO: TRAC⁻/β2M⁻

3×KO (CD70): TRAC⁻/β2M⁻/CD70⁻

Activated primary human T cells were electroporated with Cas9:gRNA RNP complexes. The nucleofection mix contained the Nucleofector™ Solution, 5×10⁶ cells, 1 μM Cas9, and 5 μM gRNA (as described in Hendel et al., Nat Biotechnol. 2015; 33(9):985-989, PMID: 26121415). For the generation of double knockout T cells (2×KO), the cells were electroporated with two different RNP complexes, each containing Cas9 protein and one of the following sgRNAs: TRAC (SEQ ID NO: 6) and β2M (SEQ ID NO: 10) at the concentrations indicated above. For the generation of triple knockout T cells (3×KO), the cells were electroporated with three different RNP complexes, each RNA complex containing Cas protein and one of the following sgRNAs: (a) TRAC (SEQ ID NO: 6), β2M (SEQ ID NO: 10), and CD70 (SEQ ID NO: 2 or 66). The unmodified versions (or other modified versions) of the gRNAs may also be used (e.g., SEQ ID NOS: 3, 7, 11, and/or 67). See also sequences in Table 6.

TABLE 6
gRNA Sequences/Target Sequences.

| Name | Unmodified Sequence | Modified Sequence |
|---|---|---|
| TRAC sgRNA | AGAGCAACAGUGCUGUGG | A*G*A*GCAACAGUGCUGU |
| | CCguuuuagagcuagaaauagcaagu | GGCCguuuuagagcuagaaauagcaa |
| | uaaaauaaggcuaguccguuaucaacuu | guuaaaauaaggcuaguccguuaucaac |
| | gaaaaaguggcaccgagucggugcUU | uugaaaaaguggcaccgagucggugcU |
| | UU | *U*U*U (SEQ ID NO: 6) |
| | (SEQ ID NO: 7) | |
| TRAC sgRNA spacer | AGAGCAACAGUGCUGUGG | A*G*A*GCAACAGUGCUGU |
| | CC (SEQ ID NO: 9) | GGCC (SEQ ID NO: 8) |
| β2M sgRNA | GCUACUCUCUCUUUCUGG | G*C*U*ACUCUCUCUUUCU |
| | CCguuuuagagcuagaaauagcaagu | GGCCguuuuagagcuagaaauagcaa |
| | uaaaauaaggcuaguccguuaucaacuu | guuaaaauaaggcuaguccguuaucaac |
| | gaaaaaguggcaccgagucggugcUU | uugaaaaaguggcaccgagucggugcU |
| | UU | *U*U*U |
| | (SEQ ID NO: 11) | (SEQ ID NO: 10) |
| β2M sgRNA spacer | GCUACUCUCUCUUUCUGG | G*C*U*Acucucucuuucu |
| | CC (SEQ ID NO: 13) | GGCC (SEQ ID NO: 12) |
| CD70 sgRNA; also | GCUUUGGUCCCAUUGGUC | G*C*U*UUGGUCCCAUUGG |
| referred to as: T7 | CCguuuuagagcuagaaauagcaagu | UCGCguuuuagagcuagaaauagcaa |
| | uaaaauaaggcuaguccguuaucaacuu | guuaaaauaaggcuaguccguuaucaac |
| | gaaaaaguggcaccgagucggugcUU | uugaaaaaguggcaccgagucggugcU |
| | UU | *U*U*U (SEQ ID NO: 2) |
| | (SEQ ID NO: 3) | |
| CD70 sgRNA spacer; | GCUUUGGUCCCAUUGGUC | G*C*U*UUGGUCCCAUUGG |
| also referred to as: | GC (SEQ ID NO: 5) | UCGC (SEQ ID NO: 4) |
| T7 | | |
| CD70 sgRNA; also | GCCCGCAGGACGCACCCA | G*C*C*CGCAGGACGCACC |
| referred to as: T8 | UAguuuuagagcuagaaauagcaagu | CAUAguuuuagagcuagaaauagca |
| | uaaaauaaggcuaguccguuaucaacuu | aguuaaaauaaggcuaguccguuaucaa |
| | gaaaaaguggcaccgagucggugcUU | cuugaaaaaguggcaccgagucggugc |
| | UU | U*U*U*U (SEQ ID NO: 66) |
| | (SEQ ID NO: 67) | |
| CD70 sgRNA spacer; | GCCCGCAGGACGCACCCA | G*C*C*CGCAGGACGCACC |
| also referred to as: | UA (SEQ ID NO: 69) | CAUA (SEQ ID NO: 68) |
| T8 | | |

About one (1) week post electroporation, cells were either left untreated or treated with phorbol myristate acetate (PMA)/ionomycin overnight. The next day cells were processed for flow cytometry (see, e.g., Kalaitzidis D et al., J Clin Invest 2017; 127(4): 1405-1413) to assess TRAC, β2M, and CD70 expression levels at the cell surface of the edited cell population. The following primary antibodies were used (Table 7):

TABLE 7
Antibodies.

| Antibody | Clone | Fluor | Catalogue # | Dilution | For 1 |
|---|---|---|---|---|---|
| TCR | BW242/ 412 | PE | 130-091-236 (Miltenyi) | 1:100 | 1 μL |
| β2M | 2M2 | PE-Cy7 | 316318 (Biolegend) | 1:100 | 1 μL |
| CD70 | 113-16 | FITC | 355105 (Biolegend) | 1:100 | l μL |

Table 8 shows highly efficient multiple gene editing. For the triple knockout cells, 80% of viable cells lacked expression of TCR, β2M, and CD70 (Table 8).

TABLE 8
Percent of viable cells lacking expression in 3KO cell populations.

| | TRAC KO | β2M KO | CD70 KO | 3KO |
|---|---|---|---|---|
| 3KO (CD70) | 99% | 79% | 99% | 80% |

To assess whether triple gene editing in T cells affects cell expansion, cell numbers were enumerated among double and triple gene edited T cells (unedited T cells were used as a control) over a two-week period of post editing. 5×10⁶ cells were generated and plated for each genotype of T cells.

Cell proliferation (expansion) continued over the post-electroporation window test. Similar cell proliferation was observed among the double (β2M−/TRAC−) and triple β2M−/TRAC−/CD70−), knockout T cells, as indicated by the number of viable cells (data not shown). These data suggest that multiple gene editing does not impact T cell health as measured by T cell proliferation.

### Example 2: Generation of Anti-CD70 CAR T Cells with Multiple Knockouts

This example describes the production of allogeneic human T cells that lack expression of the TCR gene, β2M gene, and/or CD70 gene, and express a chimeric antigen receptor (CAR) targeting CD70. These cells are designated TCR⁻/β2M⁻/CD70⁻/anti-CD70 CAR⁺ or 3×KO (CD70) CD70 CAR⁺.

A recombinant adeno-associated adenoviral vector, serotype 6 (AAV6) (MOI 50, 000) comprising the nucleotide sequence of SEQ ID NO: 43 (comprising the donor template in SEQ ID NO: 44, encoding anti-CD70 CAR comprising the amino acid sequence of SEQ ID NO: 46) was delivered with Cas9:sgRNA RNPs (1 μM Cas9, 5 μM gRNA) to activated allogeneic human T cells. The following sgRNAs were used: TRAC (SEQ ID NO: 6), β2M (SEQ ID NO: 10), and CD70 (SEQ ID NO: 2 or 66). The unmodified versions (or other modified versions) of the gRNAs may also be used (e.g., SEQ ID NOS: 3, 7, 11, and/or 67). About one (1) week post electroporation, cells were processed for flow cytometry to assess TRAC, β2M, and CD70, expression levels at the cell surface of the edited cell population. The following primary antibodies were used (Table 9):

TABLE 9
Antibodies.

| Antibody | Clone | Fluor | Catalogue # | Dilution |
|---|---|---|---|---|
| TCR | BW242/412 | PE | 130-091-236 (Miltenyi) | 1:100 |
| β2M | 2M2 | PE-Cy7 | 316318 (Biolegend) | 1:100 |
| CD70 | 113-16 | FITC | 355105 (Biolegend) | 1:100 |

T cell Proportion Assay. The proportions of CD4+ and CD8+ cells were then assessed in the edited T cell populations by flow cytometry using the following antibodies (Table 10):

TABLE 10
Antibodies.

| Antibody | Clone | Fluor | Catalogue # | Dilution |
|---|---|---|---|---|
| CD4 | RPA-T4 | BV510 | 300545 (Biolegend) | 1:100 |
| CD8 | SK1 | BV605 | 344741 (Biolegend) | 1:100 |

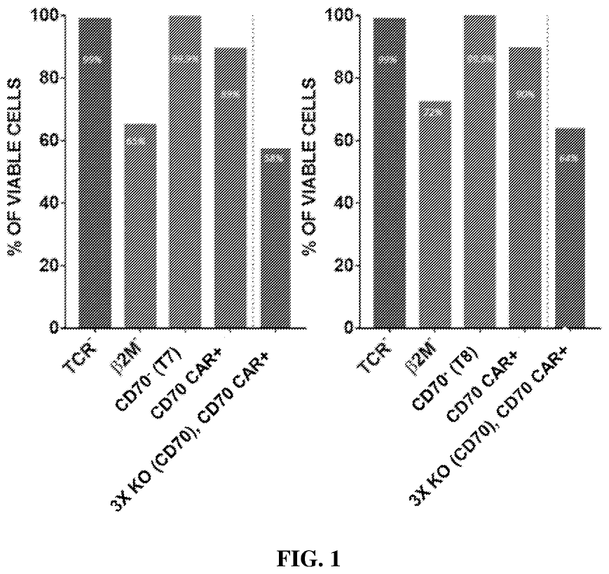

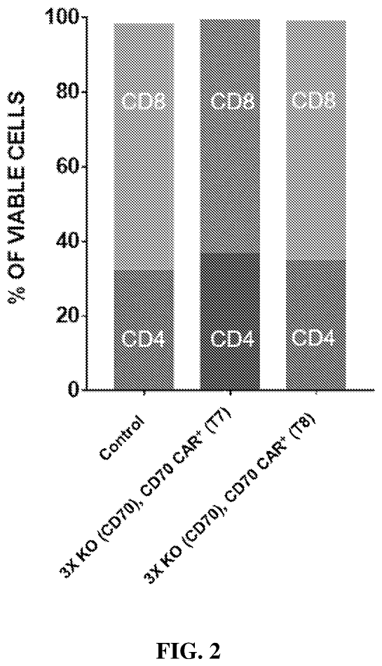

High efficiency gene editing and CAR expression was achieved in the edited anti-CD70 CAR T cell populations. In addition, editing did not adversely alter CD4/CD8 T cell populations. FIG. 1 shows highly efficient gene editing and anti-CD70 CAR expression in the triple knockout CAR T cell. More than 55% of viable cells lacked expression of TCR, β2M, and CD70, and also expressed the anti-CD70 CAR. FIG. 2 shows that normal proportions of CD4/CD8 T cell subsets were maintained in the TRAC−/β2M−/CD70−/anti-CD70 CAR+ cells, suggesting that these multiple gene edits do not affect T cell biology as measured by the proportion of CD4/CD8 T cell subsets.

### Example 3: Effect of CD70 KO on Cell Proliferation of Anti-CD70 CAR T Cells In Vitro

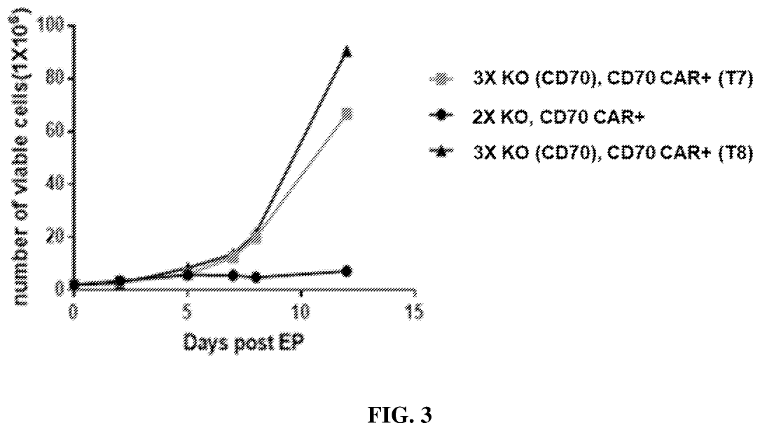

To further assess the impact of disrupting the CD70 gene in CAR T cells, anti-CD70 CAR T cells were generated as described in Example 2. Specifically, TRAC−/β2M−/CD70− anti-CD70 CAR+ T cells were generated using two different gRNAs (T7 (SEQ ID NO: 2 and T8 (SEQ ID NO: 66)). After electroporation, cell expansion was assessed by enumerating double or triple gene edited T cells over a two week period of post editing. 5×10⁶ cells were generated and plated for each genotype of T cells. Proliferation was determined by counting the number of viable cells. FIG. 3 shows that triple knockout TRAC⁻/β2M⁻/CD70⁻/anti-CD70 CAR⁺ T cells generated with either T7 or T8 gRNAs exhibited greater cell expansion relative to double knockout TRAC⁻/β2M⁻/anti-CD70 CAR⁺ T cells. These data suggest that knocking-out the CD70 gene gives a cell proliferation advantage to anti-CD70 CAR+ T cells.

### Example 4: CD70 KO Improves Cell Kill in Multiple Cell Types

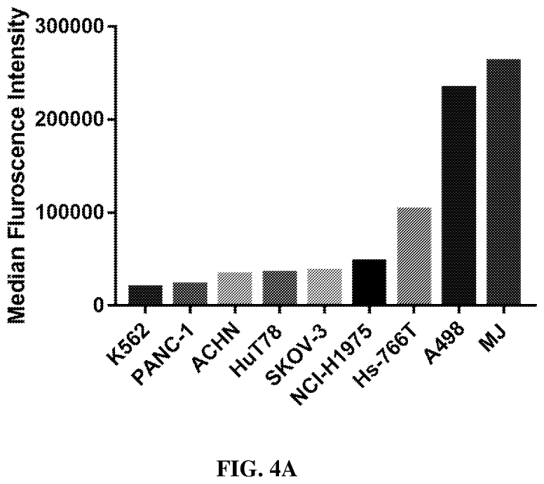

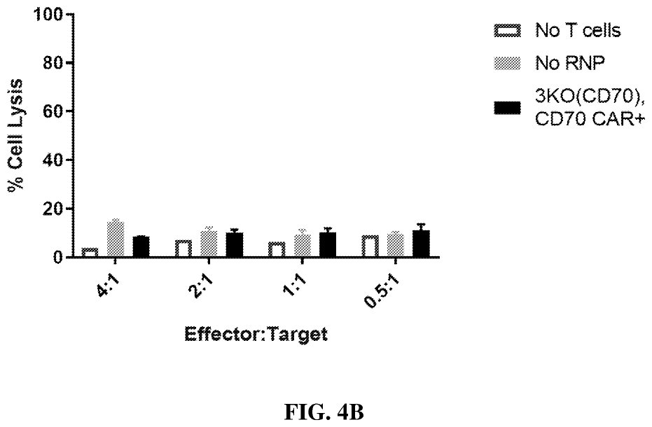

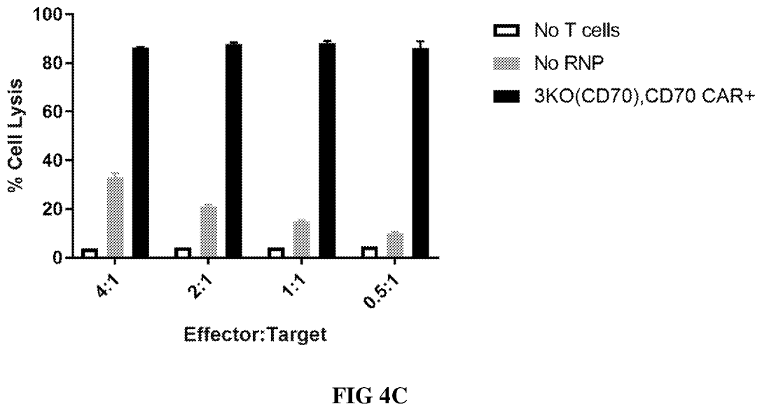

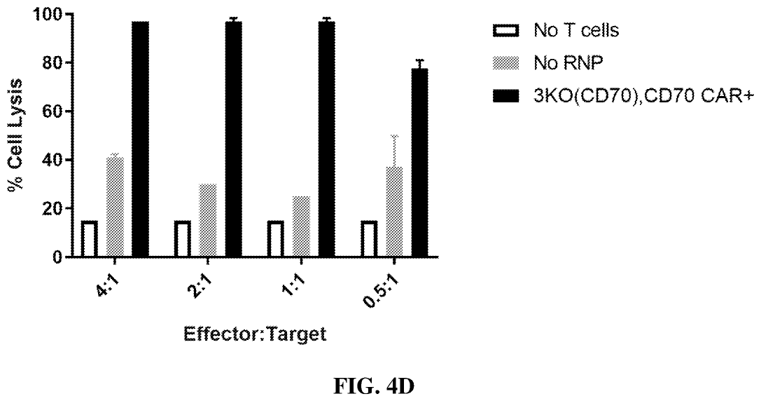

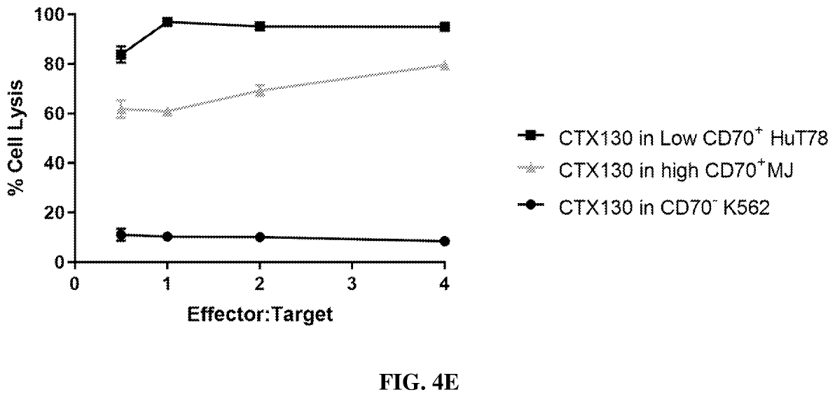

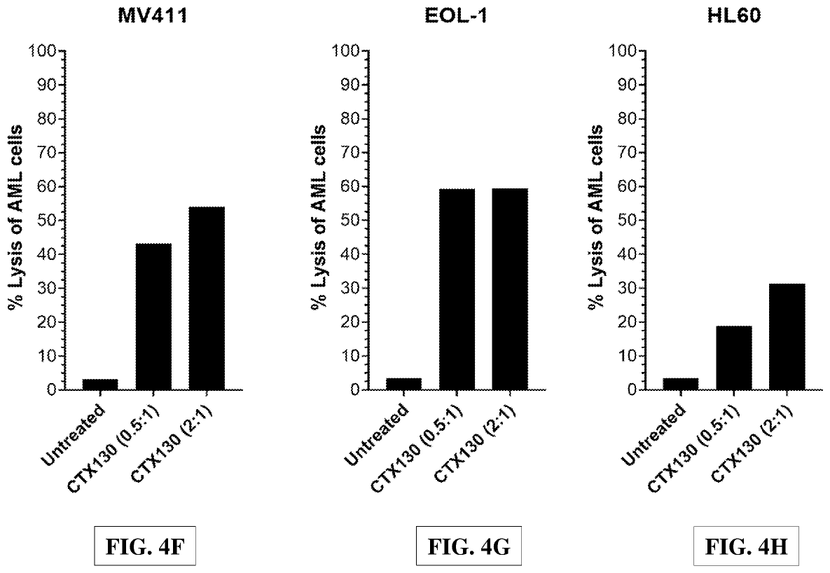

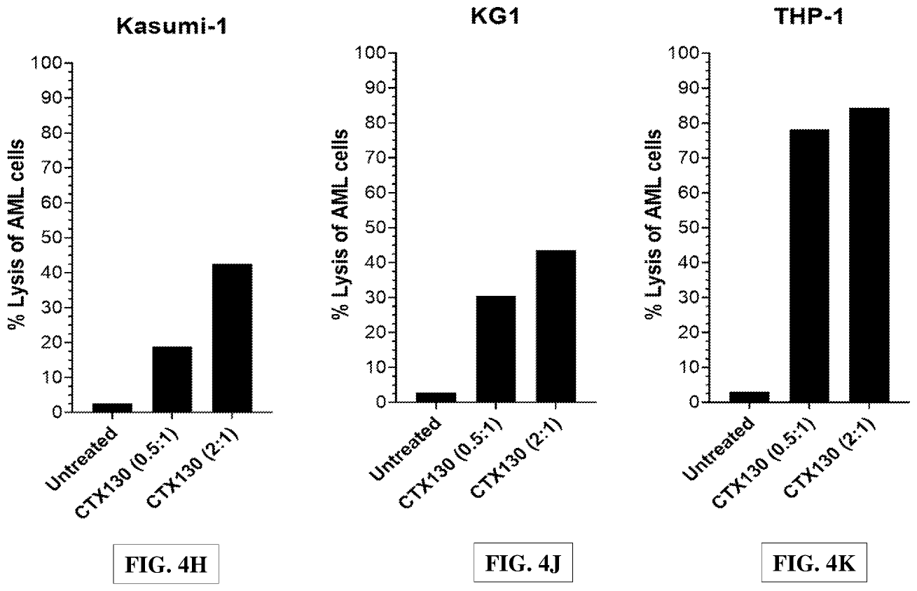

CD70 Expression in Various Cancer Cell Lines. Relative CD70 expression was measured in various cancer cell lines to further evaluate the ability of anti-CD70 CAR⁺ T cells to kill various cancer types. CD70 expression was measured by flow cytometric analysis using Alexa Fluor 647 anti-human CD70 antibody (BioLegend Cat. No. 355115). Cancer cell lines were evaluated for CD70 expression by flow cytometric analysis (Table 11A, FIG. 4A) using a FITC anti-human CD70 antibody (BioLegend Cat. No. 355105) in FIG. 4A. SKOV-3 (ovarian), HuT78 (lymphoma), NCI-H1975 (lung) and Hs-766T (pancreatic) cell lines exhibited levels of CD70 expression that were similar or higher than ACHN but lower than A498 (Table 22, FIG. 4A).

Acute myeloid Leukemia (AML) can express high levels of CD70. CD70 expression was measured in several acute myeloid leukemia cell lines by flow cytometric analysis: THP-1, MV-4-11, EOL-1, HL-60, Kasumi-1, and KG1. Table 11B shows that these cells express CD70 and can all be targeted by anti-CD70 CAR T cells, as demonstrated by the cell killing data described herein.

TABLE 11A
CD70 Expression in Cancer Cell Lines.

| Cell Line | Cancer type | Relative CD70 expression |
|---|---|---|
| A498 | Kidney Carcinoma | High |
| ACHN | Kidney (derived from metastasis) | Medium-Low |
| SK-OV-3 | Ovarian Adenocarcinoma | Medium |
| NCI-H1975 | Lung Adenocarcinoma (NSCLC) | Medium |
| Calu-1 | Lung Carcinoma | Low |
| DU 145 | Prostate Carcinoma | Low |
| SNU-1 | Gastric Carcinoma | High |
| Hs 766T | Pancreatic Carcinoma | Medium |
| MJ | Cutaneous T cell Lymphoma (CTCL) | High |
| HuT78 | Cutaneous T cell Lymphoma (CTCL; | Medium-Low |
| | Sézary syndrome) | |
| HuT102 | Cutaneous T cell Lymphoma (CTCL) | Medium |
| HH | Cutaneous T cell Lymphoma (CTCL) | Medium-Low |
| PANC-1 | Pancreatic Carcinoma | Low |
| U937 | AML: acute myeloid leukemia | No expression |
| K562 | chronic myelogenous leukemia | No expression (Negative Control) |

TABLE 11B
CD70 Expression in Leukemic Cell Lines.

| Exemplary AML Cell Lines | CD70 expression by Flow Cytometry |
|---|---|
| THP-1 | 88.9% |
| MV-4-11 | 99.9% |
| EOL-1 | 70.1% |
| HL-60 | 12.5% |
| Kasumi-1 | 19.4% |
| KG1 | 14.8% |

Cell Killing. The ability of anti-CD70 CAR+ T cells to selectively kill CD70-expressing cells was determined. A flow cytometry assay was designed to test killing of cancer cell suspension lines (e.g., K562, MM.1S, HuT78 and MJ cancer cells that are referred to as “target cells”) by 3×KO (CD70) (TRAC⁻/B2M⁻/CD70⁻) anti-CD70 CAR+ T cells. Three of the target cell lines that were used were CD70-expressing cancer cells (e.g., MM.1S, HuT78, and MJ), while a third that was used as negative control cancer cells lack CD70 expression (e.g., K562). The TRAC⁻/B2M⁻/CD70⁻/anti-CD70 CAR+ T cells were co-cultured with either the CD70-expressing MM.1S, HuT78 or MJ cell lines or the CD70-negative K562 cell line. The target cells were labeled with 5 μM efluor670 (eBiosciences), washed and seeded at a density of 50,000 target cells per well in a 96-well U-bottom plate. The target cells were co-cultured with TRAC⁻/B2M⁻/CD70⁻ anti-CD70 CAR+ T cells at varying ratios (0.5:1, 1:1, 2:1 and 4:1 CAR+ T cells to target cells) and incubated overnight. Target cell killing was determined following a 24 hour co-culture. The cells were washed and 200 μL of media containing a 1:500 dilution of 5 mg/mL DAPI (Molecular Probes) (to enumerate dead/dying cells) was added to each well. Cells were then analyzed by flow cytometry and the amount of remaining live target cells was quantified.

FIG. 4B, FIG. 4C, FIG. 4D, and FIG. 4E demonstrate selective target cell killing by TRAC−/B2M−/CD70− anti-CD70 CAR+ T cells (e.g., CTX130). A 24 hour co-culture with 3×KO (CD70) CAR+ T cells resulted in nearly complete killing of T cell lymphoma cells (HuT78), even at a low CAR+ T cell to CD70-expressing target cell ratio of 0.5:1 (FIG. 4D). Likewise, a 24 hour co-culture resulted in nearly complete killing of multiple myeloma cells (MM.1S) at all CAR+ T cell to target cell ratios tested (FIG. 4C). Similarly, a 24 hour co-culture resulted in effective cell lysis of high CD70 expressing T cell lymphoma cells (MJ) at all CAR+ T cell to target cell ratios tested. FIG. 4E shows cell lysis relative to a lower expressing CD70 T cell lymphoma cells (HuT78). Killing of target cells was found to be selective in that TRAC−/B2M−CD70−/anti-CD70 CAR+ T cells induced no killing of CD70-deficient K562 cells that was above the level of control samples (e.g., either cancer cells alone or co-culture with no RNP T cells) at any effector:target cell ratio tested (FIG. 4B). FIGS. 4F-4K demonstrate that TRAC−/B2M−/CD70− anti-CD70 CAR+ T cells (e.g., CTX130) are capable of effectively killing various CD70 expressing AML cell lines. Specifically, a 24 hour co-culture resulted in effective killing of the various acute myeloid leukemia cell lines, including MV411 (FIG. 4F), EOL-1 (FIG. 4G), HL60 (FIG. 4H), Kasumi-1 (FIG. 4H), KG1 (FIG. 4J), and THP-1 cells (FIG. 4K). In addition, the data demonstrate that the killing effect of anti-CD70 CAR T cells on acute myeloid leukemia cells increases with an increase dose of the anti-CD70 CAR T cells.

### Example 5: Efficacy of CTX130 Cells: Treatment in the Cutaneous T-Cell Lymphoma Tumor Xenograft Model

The ability of T cells expressing an anti-CD70 CAR to eliminate T cell lymphoma was evaluated in in vivo using a subcutaneous T-cell lymphoma (Hu T78 or Hh) tumor xenograft model in mice.

CRISPR/Cas9 and AAV6 were used as above (see for example, Example 2) to create human anti-CD70 CAR+ T cells that lack expression of the TCR, β2M, CD70 with concomitant expression from the TRAC locus using a CAR construct targeting CD70 (SEQ ID NO: 46). In this example activated T cells were first electroporated with 3 distinct Cas9:sgRNA RNP complexes containing sgRNAs targeting TRAC (SEQ ID NO: 6), β2M (SEQ ID NO: 10), and CD70 (SEQ ID NO: 2). The DNA double stranded break at the TRAC locus was repaired by homology directed repair with an AAV6-delivered DNA template (SEQ ID NO: 43) (encoding anti-CD70 CAR comprising the amino acid sequence of SEQ ID NO: 46) containing right and left homology arms to the TRAC locus flanking a chimeric antigen receptor cassette (−/+ regulatory elements for gene expression).

The resulting modified T cells are TRAC−/β2M−/CD70− anti-CD70 CAR+ T cells (CTX130). The ability of these anti-CD70 CAR+ T cells to ameliorate disease caused by a CD70+ T-cell lymphoma cell line was evaluated in NOG mice using methods employed by Translational Drug Development, LLC (Scottsdale, Ariz.). In brief, 12, 5-8 week old female, CIEA NOG (NOD.Cg-Prkdc^{scid}I12rg^{tm1Sug}/JicTac) mice were individually housed in ventilated microisolator cages, maintained under pathogen-free conditions, 5-7 days prior to the start of the study. Mice received a subcutaneous inoculation of 3×10⁶ T-cell lymphoma cells (HuT78 or Hh) in the right hind flank. When mean tumor size reached 25-75 mm³ (target of −50 mm³), the mice were further divided into 2 treatment groups as shown in Table 12. On Day 1, treatment group 2 received a single 200 μl intravenous dose of anti-CD70 CAR+ T cells according to Table 12.

TABLE 12
Treatment groups.

| | | | CAR+ T cell | |
|---|---|---|---|---|
| Group | CAR-T | Tumor cells | treatment (i.v.) | N |
| 1 | None | 3 × 10⁶ cells/mouse | None | 5 |
| 2 | CTX130 CART cells | 3 × 10⁶ cells/mouse | 1 × 10⁷ cells/mouse | 5 |

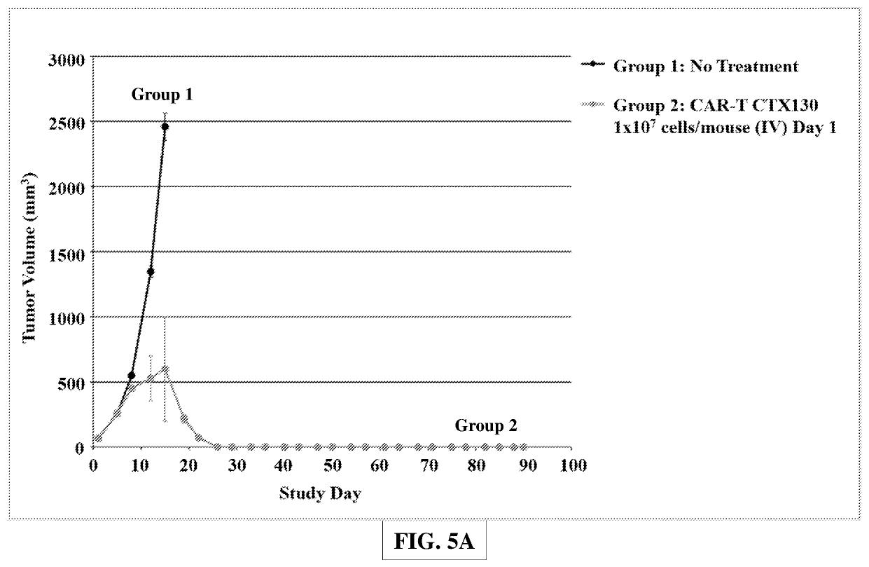

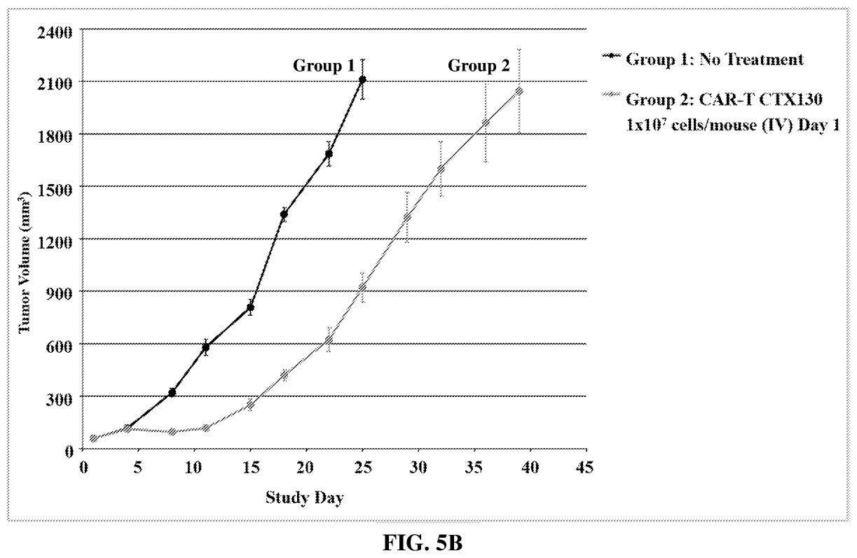

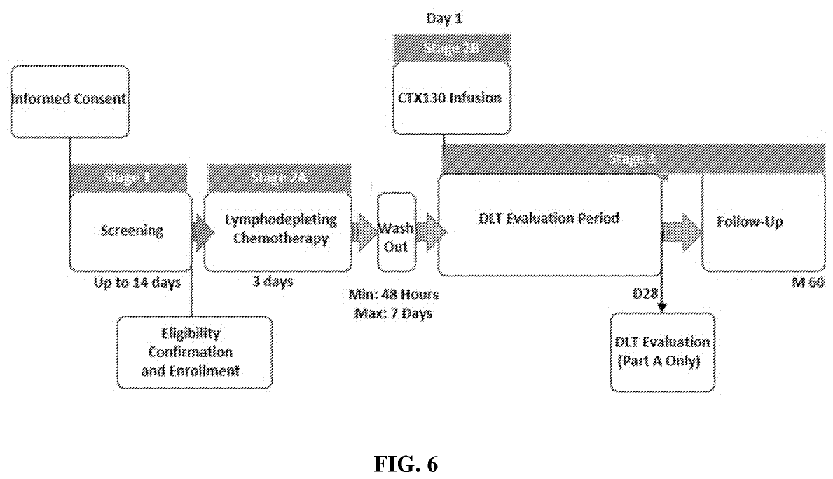

Tumor volume was measured 2 times weekly from day of treatment initiation. By Day 12 post-injection, HuT78 tumors treated with anti-CD70 CAR T cells began to show a decrease in tumor volume in 4 of the 5 treated mice (FIG. 5A). Further tumors were eliminated by day 30 and for the remainder of the study (FIG. 5A). HuT78 tumor growth was significantly inhibited over a 90 day study period (FIG. 5A). Treatment with anti-CD70 CAR+ T cells effectively slowed tumor growth of the Hh T-cell lymphoma tumors in all mice tested over a 45 day period (FIG. 5B).

These data demonstrate that anti-CD70 CAR+ cells (CTX130) inhibited growth of human CD70+ T-cell lymphoma tumors in vivo, with potent activity against established HuT78 and Hh T-cell lymphoma xenografts.

### Example 6: A Phase 1, Open-Label, Multicenter, Dose Escalation and Cohort Expansion Study of the Safety and Efficacy of Allogeneic CRISPR-Cas9 Engineered T Cells (CTX130) in Adult Subjects with T Cell or B Cell Malignancies

CTX130 is a CD70-directed T cell immunotherapy comprised of allogeneic T cells that are genetically modified ex vivo using CRISPR-Cas9 (clustered regularly interspaced short palindromic repeats/CRISPR-associated protein 9) gene editing components (single guide RNAs [sgRNAs] and Cas9 nuclease). The modifications include targeted disruption of the T-cell receptor alpha constant (TRAC), beta 2-microglobulin (B2M), and CD70 loci and the insertion of an anti-CD70 chimeric antigen receptor (CAR) transgene into the TRAC locus via an adeno-associated virus (AAV) expression cassette. The anti-CD70 CAR (SEQ ID NO: 46) is composed of an anti-CD70 single-chain variable fragment (SEQ ID NO: 48) derived from a previously characterized anti-CD70 hybridoma IF6, a CD8 transmembrane domain (SEQ ID NO: 54), a 4-1BB co-stimulatory domain (SEQ ID NO: 57), and a CD3ζ signaling domain (SEQ ID NO: 61).

In this study, eligible human patients receive an intravenous (IV) infusion of CTX130 following lymphodepleting (LD) chemotherapy.

### 1. Study Population

Dose escalation (Part A) includes adult subjects with the following relapsed/refractory T cell or B cell malignancies: (a) Peripheral T cell lymphoma, not otherwise specified (PTCL-NOS), (b) Anaplastic large cell lymphoma (ALCL), (c) Sézary syndrome (SS) including mycosis fungoides (MF), (d) Adult T cell leukemia/lymphoma (ATLL), leukemic and lymphomatous subtypes, (e) Angioimmunoblastic T cell lymphoma (AITL), and (f) Diffuse large B cell lymphoma (DLBCL). Cohort expansion (Part B) includes subjects with DLBCL and the same inclusion and exclusion criteria for enrollment in Part A, as well as subjects with T cell lymphomas described herein.

Subjects to be treated in this study may also include those having T or B cell lymphomas, for example, CTCL (include Mycosis fungoides Stage IIb and higher, including in transformation to large cell lymphoma, Sezary Syndrome); PTCL: AITL, ALCL (Alk positive and negative), ATLL, except the smoldering subtype, and PTCL-NOS); and DLBCL after failed autologous CD19-directed CAR T cell therapy.

### 2. Study Purpose and Rationale

The purpose of the Phase 1 dose escalation study is to evaluate the safety and efficacy of anti-CD70 allogeneic CRISPR-Cas9 engineered T cells (CTX130 cells) in subjects with relapsed or refractory B cell malignancies.

There is an unmet medical need in subjects with the selected and described T or B cell lymphomas (e.g., those disclosed herein). The selected T or B cell malignancies are reported to have a high expression of CD70, and therefore, are a potential target for CAR T cell-directed therapies (Baba et al., (2008) J Virol 82 3843-52; Lens et al., (1999) Br J Hematol 106, 491-503; McEarchern et al., (2007) Blood 109, 1185-92; Shaffer et al., (2011) Blood 117, 4304-14).

Although CAR T cell therapy has led to tremendous clinical success, the approved products are autologous and require patient-specific cell collection and manufacturing. These challenges have led to a significant proportion (approximately 30% in 1 study) of subjects enrolled that never received the autologous CAR T cell product (Schuster et al., (2019) N Engl J Med 380, 45-56). In addition, the heterogeneous nature of each autologous product has made it challenging to demonstrate correlation between CAR T cell dose, toxicity, and/or response in most of the disease indications studied (Mueller et al., (2017) Blood 130, 2317-2325). Recent data suggest that the starting material, specifically the immunophenotype of isolated T cells, may have an impact on disease response (Fraietta et al., (2018) Nat Med 24, 563-71). These findings underpin the benefit of an allogeneic CAR T treatment approach for those patients when in need of an urgent, cytoreductive therapy.

CTX130 is manufactured from the T cells of healthy donors, which is intended to result in consistent CAR expression and immunophenotypes across manufacturing runs. Additionally, the manufacturing process initiated from healthy donor cells greatly diminishes the risk of unintentionally transducing malignant T cells during treatment. The recently reported case of a subject with ALL who relapsed with malignant B cells transduced with CAR T cells further underscores this potential risk of a lentiviral approach in which CAR insertion is not coupled to TCR disruption (Ruella et al., (2018) Nat Med 24, 1499-503). Individual subject manufacturing failures, scheduling complexities, toxicity associated with bridging chemotherapy, and the risks of leukapheresis to the subject do not apply to allogeneic CAR T cell products. The ability to administer CTX130 immediately allows for subjects to receive the product in a timely fashion and helps subjects avoid the need for bridging chemotherapy.

Autologous CAR T cells generated from patients with advanced, relapsed malignancies might be prone to early exhaustion (Fraietta et al., (2018) Nat Med 24, 563-71; Mackall, (2019) Cancer Research, AACR annual meeting, Abstract PL01-05; Riches et al., (2013) Blood 121, 1612-21). The use of healthy donor T lymphocytes as the basis for multi-edited allogeneic CAR T cells becomes possible due to the highly precise editing tool CRISPR-Cas9.

The 4 editing steps applied to CTX130 address the safety and efficacy in the following manner:
Safety: Deletion of the TRAC locus to disrupt the endogenous TCR and its interactions with the host MHC system to suppress graft versus host disease (GvHD).
T cell activity: Insertion of the CD70-targeting CAR construct, deletion of the B2M locus, and deletion of the CD70 locus.

CRISPR-Cas9 allows the coupling of the introduction of the CAR construct as the locus of the deleted through homologous recombination. The delivery and precise insertion of the CAR at the TRAC genomic locus using an AAV-delivered DNA donor template and HDR contrasts with the random insertion of genetic material using other common transduction methods such as lentiviral and retroviral transduction. CAR gene insertion at the TRAC locus results in elimination of TCR in nearly all cells expressing the CAR. While CRISPR-Cas9-mediated disruption of the endogenous TCR can significantly reduce or eliminate the risk of GvHD, the disruption of MHC class I proteins is hypothesized to increase CAR T cell persistence. Deletion of the CD70 locus is intended to increase the persistence of CTX130 and to reduce potential fraternization through elevated expression on activated CAR T cells.

### 3. Study Objectives

Primary Objective, Part A (Dose escalation): To assess the safety of escalating doses of CTX130 in subjects with relapsed/refractory T or B cell malignancies and to determine the recommended Part B dose (RPBD).

Primary Objective, Part B (Cohort expansion): To assess the efficacy of CTX130 in subjects with DLBCL (e.g., those who failed an earlier autologous CD19 directed CAR-T therapy), as well as other types of T cell lymphoma disclosed above, as measured by objective response rate (ORR) according to Lugano response criteria (Cheson et al., (2014) J Clin Oncol 32, 3059-68).

Secondary Objectives (Parts A and B): To assess activity of CTX130 including time to response (TTR), duration of response (DoR), progression free survival (PFS), overall survival (OS), disease control rate (DCR), time to progression (TTP) over time; to describe and assess adverse events (AEs) of interest, including cytokine release syndrome (CRS) and graft versus host disease (GvHD); and to characterize pharmacokinetics (expansion and persistence) of CTX130 in blood.

Exploratory Objectives (Parts A and B): To identify genomic, metabolic, and/or proteomic biomarkers that are associated with disease, clinical response, resistance, or safety; to characterize pharmacodynamic activity potentially related to clinical response; to further describe the kinetics of efficacy of CTX130, and to describe the effect of CTX130 on patient-reported outcomes (PRO).

### 4. Study Eligibility

### 4.1 Inclusion Criteria

To be considered eligible to participate in this study, a subject must meet all the inclusion criteria listed below:

1. ≥18 years of age and body weight≥60 kg.

2. Able to understand and comply with protocol-required study procedures and voluntarily sign a written informed consent document.

3. For subjects with T cell lymphoma only the following are enrolled:
Confirmed diagnosis of a T cell malignancy, including the following subsets:
   a) PTCL-NOS,
   b) ALCL,
   c) SS including MF≥Stage IIB (e.g., who may be in need of transplant),
   d) Leukemic and lymphomatous subtypes of ATLL,
   e) Angioimmunoblastic T-cell lymphoma (AITL). In some instances, subjects who have had any effusion prior to or during the screening period may be excluded.
Subjects with PTCL-NOS, ATLL, or AITL should have failed ≥1 lines of systemic therapy.
Subjects with ALCL should have failed, be ineligible for, or have refused combination chemotherapy and/or therapy with brentuximab vedotin in combination or as a single agent.
   Subjects with anaplastic lymphoma kinase negative (ALK⁻) ALCL should have failed one prior line of therapy.
   Subjects with anaplastic lymphoma kinase positive (ALK⁺) ALCL should have failed 2 prior lines of therapy.
Subjects with mycosis fungoides (MF) or Sézary Syndrome (SS) must have failed at least have failed at least 2 of the following systemic therapies: brentuximab vedotin, romidepsin (or other indicated histone deacetylase [HDAC] inhibitors), pralatrexate, mogamulizumab, or chemotherapy. If mogamulizumab was the last therapy prior to enrollment, there must be at least 3 months between the last dose of mogamulizumab and the infusion of CTX130.

4. For subjects with B Cell lymphoma: DLBCL in subjects who are eligible for autologous CD19 CAR T cell therapy but have failed a treatment attempt with it.

5. Subjects must have CD70-expressing tumors as determined by laboratories meeting applicable local requirements (e.g., Clinical Laboratory Improvement Amendments [CLIA] or equivalent for non-US locations) by either:
CD70 positivity (≥10% of cells) by immunohistochemistry (IHC) in tissue collected by excisional or core biopsy of a representative tumor lesion.
CD70 positivity (10% of cells) by flow cytometry in tumor cells defined by immunophenotyping collected in the peripheral blood or bone marrow at screening.

6. Be willing to provide tissue from a newly obtained core or excisional biopsy of a tumor lesion at screening unless a biopsy performed within 3 months prior to enrollment and after the last systemic or targeted therapy post progression is available.

7. Eastern Cooperative Oncology Group (ECOG) performance status of 0-1 (see Table 13).

8. Meets criteria to undergo LD chemotherapy and CAR T cell infusion described herein.

9. Adequate organ function:
Renal: creatinine clearance (CrCl)≥50 mL/min
Liver:
   Aspartate aminotransferase (AST) or alanine aminotransferase (ALT)<3× upper limit of normal (ULN).
   Total Bilirubin<2×ULN (for Gilbert's syndrome: Total Bilirubin<3 mg/dL and normal conjugated bilirubin).
Cardiac: Hemodynamically stable and left ventricular ejection fraction (LVEF)≥45% by echocardiogram.
Pulmonary: Oxygen saturation level on room air>92% per pulse oximetry.
Hematologic: Platelet count>25,000/mm³ and absolute neutrophil count>500/mm³.

10. Female patients of childbearing potential (postmenarcheal, has an intact uterus and at least 1 ovary, and is less than 1 year postmenopausal) must agree to use acceptable method of highly effective contraception from enrollment through at least 12 months after CTX130 infusion.

11. Male patients must agree to use acceptable highly effective methods of contraception from enrollment through at least 12 months after CTX130 infusion.

TABLE 13
ECOG Performance Status Scale.

| Grade | Description |
|---|---|
| 0 | Fully active, able to carry on all pre-disease performance without restriction |
| 1 | Restricted in physically strenuous activity but ambulatory and able to carry |
| | out work of a light or sedentary nature, e.g., light house work, office work |
| 2 | Ambulatory and capable of all self-care but unable to carry out any work |
| | activities; up and about more than 50% of waking hours |
| 3 | Capable of only limited self-care; confined to bed or chair more than 50% of |
| | waking hours |
| 4 | Completely disabled; cannot carry on any self-care; totally confined to bed or |
| | chair |
| 5 | Dead |

### Developed by the Eastern Cooperative Oncology Group, Robert L. Comis, MD, Group Chair (Oken et al., (1982) Am J Clin Oncol, 5, 649-655).

### 4.2 Exclusion Criteria

To be eligible to participate in this study, a subject must not meet any of the exclusion criteria listed below:

1. Prior allogeneic stem cell transplant (SCT).

2. Less than 60 days from autologous SCT at time of screening and with unresolved serious complications.

3. Prior treatment with anti-CD70 targeting agents.

4. For subjects with DLBCL, prior treatment with CAR T cells or other modified T or natural killer (NK) cells except autologous CD19 CAR T cells.

5. Known contraindication to any LD chemotherapy agent(s) or any of the excipients of CTX130 product.

6. T cell or B cell lymphomas with a present or past malignant effusion that is or was symptomatic.

7. Clinical signs of hemophagocytic lymphohistiocytosis (HLH): A combination of fever, bicytopenia, hypertriglyceridemia or hypofibrinogenemia and ferritin>500 μg/L.

8. Active central nervous system (CNS) manifestation of underlying disease in screening imaging.

9. History or presence of clinically relevant central nervous system (CNS) pathology such as seizure, stroke, severe brain injury, cerebellar disease, myelopathy (e.g., tropical spastic paraparesis), history of posterior reversible encephalopathy syndrome (PRES) with prior therapy, or another condition that may increase CAR T-related toxicities.

10. Unstable angina, arrhythmia, or myocardial infarction within 6 months prior to screening.

11. Uncontrolled, acute life-threatening bacterial, viral, or fungal infection.

12. Positive for presence of human immunodeficiency virus type 1 or 2 (HIV-1 or HIV-2), or active hepatitis B virus or hepatitis C virus infection. Subjects with prior history of hepatitis B or C infection who have documented undetectable viral load (by quantitative polymerase chain reaction or nucleic acid testing) are permitted.

13. Previous or concurrent malignancy, except those treated with curative approach who have been in remission for >12 months without requiring systemic therapy (antihormonal therapy accepted).

14. Primary immunodeficiency disorder or active autoimmune disease requiring steroids and/or other immunosuppressive therapy.

15. Prior solid organ transplantation.

16. Prior use of antitumor agents, including radiotherapy, 14 days prior to enrollment. For investigational agents, washout time needs to be discussed with the medical monitor. Use of physiological doses of steroids is permitted for subjects previously on steroids. Intrathecal prophylaxis for subjects with ATLL is permitted if indicated. Subjects with ATLL receiving the RANKL inhibitor denosumab should be on therapy for at least 4 weeks and must have stabilized corrected serum calcium levels; and are excluded if serum calcium level is >11.5 mg/dL or >2.9 mmol/L, or ionized calcium level is >1.5 mmol/L. Use of CCR-4 directed antibodies like mogamulizumab are prohibited 3 months prior to CTX130 infusion.

17. Diagnosis of significant psychiatric disorder that could seriously impede the patient's ability to participate in the study.

18. Received live vaccines or herbal medicines as part of traditional Chinese medicine or non-over-the-counter herbal remedies within 28 days prior to enrollment.

19. Pregnant or breastfeeding females.

### 5. Study Design

### 5.1 Investigational Plan

This is an open-label, multi-cohort, multi-center, dose escalation Phase 1 study in subjects≥18 years of age with relapsed or refractory T or B cell malignancies. The study is divided into 2 parts: dose escalation (Part A) followed by cohort expansion (Part B).

In Part A, dose escalation begins in adult subjects with 1 of the following:

1. T cell malignancies:
Subjects with PTCL-NOS, leukemic and lymphomatous ATLL, or AITL should have failed ≥1 lines of systemic therapy.
Subjects with ALCL should have failed, be ineligible for, or have refused combination chemotherapy and/or therapy with brentuximab vedotin.
   Subjects with ALK− ALCL should have failed 1 prior line of therapy.
   Subjects with ALK+ ALCL should have failed 2 prior lines of therapy.
Subjects with MF or SS should have failed at least 1 prior therapy. Subjects with SS should have failed prior systemic therapy including mogamulizumab treatment, if indicated. If mogamulizumab was the last therapy prior to enrollment, there must be a period of at least 3 months between the last dose of mogamulizumab and the infusion of CTX130.

2. B cell malignancy:
DLBCL in subjects who failed a treatment attempt with autologous CD19 CAR T cell therapy.

Dose escalation is performed according to the criteria described herein.

In Part B, an expansion cohort is initiated to further assess the safety and efficacy of CTX130 at the RPBD in subjects with DLBCL who have failed a prior treatment attempt with autologous CD19 CAR T cells. Subjects with DLBCL are enrolled in Part B according to the same inclusion and exclusion criteria needed for enrollment in Part A. This expansion is designed to reject an ORR of less than 18% in patients post autologous CD19 CAR T therapy.

### 5.1.1 Study Design

The study is divided into 2 parts: dose escalation (Part A) followed by cohort expansion (Part B). Both parts of the study consist of 3 main stages: screening, treatment, and follow-up. A schematic depiction of the study schema is shown in FIG. 6.

The 3 main stages of the study are as follows:
Stage 1—Screening to determine eligibility for treatment (up to 14 days).
Stage 2—Treatment.
   Stage 2A—LD chemotherapy: Co-administration of fludarabine 30 mg/m² and cyclophosphamide 500 mg/m² IV daily for 3 days. Both agents are started on the same day and administered for 3 consecutive days. LD chemotherapy must be completed at least 48 hours (but no more than 7 days) prior to CTX130 infusion.
   Stage 2B—CTX130 infusion: Administered at least 48 hours (but no more than 7 days) after completion of the 3-day course of LD chemotherapy.
   Clinical eligibility—Subjects' clinical eligibility should be reconfirmed according to the criteria provided herein prior to both the initiation of LD chemotherapy and infusion of CTX130.
Stage 3—Follow up (5 years after the last CTX130 infusion)

During the post-CTX130 infusion period, subjects are monitored for acute toxicities (Days 1-28), including CRS, neurotoxicity, GvHD, and other AEs. Toxicity management guidelines are described herein. During Part A (dose escalation), all subjects are hospitalized for the first 7 days following CTX130 infusion, or longer if required by local regulation or site practice. In both Part A and Part B, subjects must remain within proximity of the investigative site (i.e., 1-hour transit time) for 28 days after CTX130 infusion.

After the acute toxicity observation period, subjects are subsequently followed for up to 5 years after CTX130 infusion with physical exams, regular laboratory and imaging assessments, and AE assessments. After completion of this study, subjects are required to participate in a separate long-term follow-up study for an additional 10 years to assess long-term safety and survival.

### 5.2 CTX130 Dose Escalation

CTX130 cells are administered IV using a flat dosing schema based on the number of CAR+ T cells. Dose levels evaluated in this study are presented in Table 14. A dose limit of 1×10⁵ TCR+ cells/kg may be imposed for all dose levels.

TABLE 14
Dose Escalation of CTX130.

| Dose Level | Total CAR+ T-Cell Dose |
|---|---|
| −1 (de-escalation) | 1 × 10⁷ |
| 1 | 3 × 10⁷ |
| 2 | 1 × 10⁸ |
| 3 | 3 × 10⁸ |
| 4 | 9 × 10⁸ |

Dose escalation is performed using a standard 3+3 design in which 3 to 6 subjects are enrolled at each dose level depending on the occurrence of dose limiting toxicities (DLTs), as defined herein.

Dose escalation is performed according to the following rules:
If 0 of 3 subjects experience a DLT, escalate to the next dose level.
If 1 of 3 subjects experiences a DLT, expand the current dose level to 6 subjects.
   If 1 of 6 subjects experiences a DLT, escalate to the next dose level.
   If ≥2 of 6 subjects experience a DLT:
      If in Dose Level −1, evaluate alternative dosing schema or declare inability to determine recommended dose for Part B cohort expansion.
      If in Dose Level 1, de-escalate to Dose Level −1.
      If in Dose Level 2-4, declare previous dose level the maximum tolerated dose (MTD).
If ≥2 of 3 subjects experience a DLT:
   If in Dose Level −1, evaluate alternative dosing schema or declare inability to determine the recommended dose for Part B cohort expansion.
   If in Dose Level 1, decrease to Dose Level −1.
   If in Dose Level 2-4, declare previous dose level the MTD.
Intermediate dosing between DL2 and DL3 will be allowed, for example, 1.5×10⁸ CAR⁺ CTX130 cells.
Intermediate dosing between DL3 and DL4 will be allowed, for example, 4.5×10⁸, 6×10⁸, or 7.5×10⁸ CAR+ CTX130 cells
No dose escalation beyond highest dose listed in Table 14.

### 5.2.1 Dose-limiting Toxicity (DLT) Definitions

The DLT evaluation period begins with CTX130 infusion and last for 28 days. In all Dose Levels (−1 to 4), subjects 1 through 3 are treated in a staggered manner, such that a subject only receives CTX130 once the previous subject has completed the DLT evaluation period (i.e., staggered by at least 28 days). Dosing between each dose level can also be staggered by at least 28 days.

Subjects must receive CTX130 to be evaluated for DLT. If a subject discontinues the study any time prior to CTX130 infusion for reasons other than toxicity, the subject is not to be evaluated for DLT and a replacement subject is to be enrolled at the same dose level as the discontinued subject. If a DLT-evaluable subject (i.e., a subject that has been administered CTX130) has signs or symptoms of a potential DLT, the DLT evaluation period may be extended to allow for improvement or resolution before a DLT is declared.

Toxicities are graded and documented according to NCI Common Terminology Criteria for Adverse Events (CTCAE) version 5.0, except for CRS (ASTCT criteria; American Society for Transplantation and Cellular Therapy criteria; Lee criteria), neurotoxicity (ICANS criteria; immune effector cell-associated neurotoxicity syndrome criteria, CTCAE version 5.0; Lee criteria), and GvHD (MAGIC criteria; Mount Sinai Acute GvHD International Consortium criteria; Harris et al., (2016) Biol Blood Marrow Transplant 22, 4-10). AEs that have no plausible causal relationship with CTX130 is not to be considered DLTs.

DLTs are defined as:
A. Grade 4 CRS
B. Grade≥2 GvHD that is steroid-refractory (e.g., progressive disease after 3 days of steroid treatment [e.g., 1 mg/kg/day], or having no response after 7 days of treatment). GvHD that is not steroid-refractory and resolves to Grade 1 within 14 days are not to be defined as a DLT (GvHD grading is provided in Table 34).
C. Grade 3 or 4 neurotoxicity (based on ICANS criteria).
D. Death during the DLT period (except due to disease progression).
E. Any Grade 4 hematologic toxicity that does not recover to ≤Grade 2 within 28 days.
F. Any Grade≥3 CTX130-treatment emergent vital organ toxicity (e.g., pulmonary, cardiac) of any duration that is not related to the underlying malignancy or its progression is considered a DLT with the following exceptions:

| Exceptions | Criteria |
|---|---|
| #1 | Any Grade 3 CRS according to the CRS Grading System (Table 29) that |
| | improves to Grade ≤2 with appropriate medical intervention within 72 hours. |
| #2 | Grade 3 or 4 fever resolving within 72 hours |
| | with appropriate medical intervention. |
| #3 | Grade 3 or 4 fatigue lasting <7 days. |
| #4 | Any Grade 3 or 4 abnormal liver function tests |
| | that improve to Grade ≤2 within 7 days. |
| #5 | Grade 3 or 4 renal insufficiency that improves to Grade ≤2 within 7 days. |
| #6 | Death due to disease progression. |
| #7 | Grade 3 or 4 thrombocytopenia or neutropenia is assessed retrospectively. After |
| | at least 6 subjects are infused, if ≥50% of subjects have prolonged cytopenias |
| | (i.e., lasting more than 28 days post infusion), |
| | dose escalation is suspended pending |
| | Grade ≥3 cytopenias that were present at the start of LD |
| | chemotherapy may not be considered a DLT |
| | and identification of another etiology. |

### 6. Study Procedures

Both the dose escalation and expansion parts of the study consists of 3 distinct stages: (1) screening and eligibility confirmation, (2) LD chemotherapy and CTX130 infusion, and (3) follow-up. During the screening period, subjects are assessed according to the eligibility criteria described herein. After enrollment, subjects receive LD chemotherapy, followed by infusion of CTX130. After completing the treatment period, subjects are assessed for tumor response, disease progression, and survival. Throughout all study periods, subjects are regularly monitored for safety.

A complete schedule of assessments is provided in Table 15 and Table 16. Missed evaluations should be rescheduled and performed as close to the originally scheduled date as possible. An exception is made when rescheduling becomes, in the healthcare practitioner's opinion, medically unnecessary or unsafe because it is too close in time to the next scheduled evaluation. In that case, the missed evaluation should be abandoned.

For the purposes of this protocol, there is no Day 0. All visit dates and windows are to be calculated using Day 1 as the date of CTX130 infusion.

### 6.2 Immune Effector Cell-Associated Encephalopathy (ICE) Assessment

Neurocognitive assessment is to be performed using ICE assessment. The ICE assessment tool is a slightly modified version of the CARTOX-10 screening tool, which now includes a test for receptive aphasia (Neelapu et al., (2018) Nat Rev Clin Oncol 15, 47-62). ICE assessment examines various areas of cognitive function: orientation, naming, following commands, writing, and attention (see Table 17).

TABLE 17
ICE Assessment.

| | | Maximum |
|---|---|---|
| Domain | Assessment | Score |
| Orientation | Orientation to year, month, city, hospital | 4 points |
| Naming | Name 3 objects (e.g., point to clock, pen, button) | 3 points |
| Following | Ability to follow commands | 1 point |
| command | (e.g., “Show me 2 fingers” or | |
| | “Close your eyes and stick out your tongue”) | |
| Writing | Ability to write a standard sentence (includes a noun and verb) | 1 point |
| Attention | Ability to count backward from 100 by 10 | 1 point |

ICE score are reported as the total number of points (0-10) across all assessments.

ICE assessment is performed at screening, before administration of CTX130 on Day 1, and on Days 2, 3, 5, 7, and 28. If a subject experiences CNS symptoms, ICE assessment should continue to be performed approximately every 2 days until resolution of symptoms. To minimize variability, whenever possible the assessment should be performed by the same research staff member who is familiar with or trained in administration of the ICE assessment tool.

### 6.3 Patient-Reported Outcomes

Five PRO surveys, the European Organisation for Research and Treatment of Cancer (EORTC) QLQ-C30, the EuroQol EQ-5D-5L questionnaires, Functional Assessment of Cancer Therapy-General (FACT-G), Skindex-29 questionnaire for SS and MF, and Dermatology Life Quality Index (DLQI) questionnaire for SS and MF are administered according to the schedule in Table 15 and Table 16. Questionnaires should be completed (self-administered in the language the subject is most familiar) before clinical assessments are performed.

The EORTC QLQ-C30 is a questionnaire designed to measure quality of life in cancer. It is composed of 5 multi-item functioning scales (physical, role, social, emotional, and cognitive function), 3 symptom scales (fatigue, nausea, pain) and additional single symptom items (financial impact, appetite loss, diarrhea, constipation, sleep disturbance, and quality of life). The EORTC QLQ-C30 is validated and has been widely used among cancer patients (Wisloff et al., (1996) Br J Haematol 92, 604-13; Wisloff and Hjorth, (1997) Br J Haematol 97, 29-37). It is scored on a 4-point scale (1=not at all, 2=a little, 3=quite a bit, 4=very much). The EORTC QLQ-C30 instrument also contains 2 global scales that use 7-point scale scoring with anchors (1=very poor and 7=excellent).

The EQ-5D-5L is a generic measure of health status and contains a questionnaire that assesses 5 domains, including mobility, self-care, usual activities, pain/discomfort, and anxiety/depression, plus a visual analog scale. EQ-5D-5L has been used in conjunction with QLQ-C30 (Moreau et al., (2019) Leukemia 33, 12:2934-2946).

The FACT-G is a validated 27-item instrument that measures the impacts of cancer therapy in 4 domains: physical, social/family, emotional, and functional well-being. The FACT-G total score is based on all 27 items and ranges from 0 to 108, with higher scores indicating better quality of life (Cella et al., (1993) J Clin Oncol 11, 570-9).

The Skindex-29 is designed to measure the effects of skin disease on quality of life in 3 domains: symptoms (7 items), emotions (10 items), and functioning (12 items). All responses are transformed to a linear scale of 100, varying from 0 (no effect) to 100 (effect experienced all the time). Scores are reported as 3 scale scores, corresponding to the 3 domains; a scale score is the average of a patient's responses to items in a given domain (Chren, (2012) Dermatol Clin 30, 231-6).

TABLE 15
Schedule of Assessments: Screening to Month 24.

| | Assessment | | | | | | | | | | | | |
|---|---|---|---|---|---|---|---|---|---|---|---|---|---|
| | | | CTX130 | Follow-Up | | | | | | | | | |
| | | | Infusion ³ | | | | | | | | | | |
| | | | Day | | | | | | | | | | |
| | | | | | | | Day | Day | Day | Day | Day | M2 | M3 |
| | | LD | Day | Day | Day | Day | 7 ± | 10 ± | 14 ± | 21 ± | 28 ± | (D56) ± | (D84) ± |
| | Screening ¹ | Chemo ² | 1⁴ | 2 | 3 | 5 | 2 d | 2 d | 2 d | 2 d | 2 d | 7 d | 7 d |
| Eligibility | X | X | X | | | | | | | | | | |
| confirmation ⁵ | | | | | | | | | | | | | |
| informed consent | X | | | | | | | | | | | | |
| Medical history ⁶ | X | | | | | | | | | | | | |
| Physical exam ⁷ | X | X | X | X | X | X | X | X | X | X | X | X | X |
| Vital signs ⁸ | X | X | X | X | X | X | X | X | X | X | X | X | X |
| Height, weight ⁹ | X | X | X | | | | X | | X | | X | | |
| Pregnancy test ¹⁰ | X | X | | | | | | | | | X | X | X |
| ECOG status | X | X | X | | | | | | | | X | | X |
| Echocardiogram | X | | | | | | | | | | | | |
| 12-lead ECG ¹¹ | X | X | X | | | | | | | | X | | |
| ICE assessment ¹² | X | | X | X | X | X | X | | | | X | | |
| PRO ¹³ | X | | X | | | | X | | X | X | X | | X |

| Con meds ¹⁴ | Continuous | |
|---|---|---|
| AEs ¹⁵ | Continuous | |
| Hospital utilization | | Continuous |

B and T Cell Lymphoma Disease/Response Assessments

| | | | | | |
|---|---|---|---|---|---|
| Whole body PET/CT | X | | | X | X |
| scan ¹⁶ | | | | | |
| Brain MRI ¹⁶ | X | | | | |
| Cutaneous | | X¹⁷ | X | X | X |
| assessment | | | | | |
| for all T-cell | | | | | |
| lymphomas ¹⁷ | | | | | |
| Tumor biopsy ¹⁸ | X | | X | X | |
| Peripheral blood | X | | X | X | X |
| assessment ¹⁹ | | | | | |
| BM aspirate/ | X | | | X²⁰ | |
| biopsy ²⁰ | | | | | |
| MRD for DLBCL ²¹ | X | | X | X | X |
| Disease response | X | | | X | X |
| assessment for | | | | | |
| ATLL ²² | | | | | |

Laboratory Assessments (Local) ⁹

| | | | | | | | | | | | | | |
|---|---|---|---|---|---|---|---|---|---|---|---|---|---|
| CD70 expression ²³ | X | | | | | | | | | | | | |
| CBC w/ | X | X | X | X | X | X | X | X | X | X | X | X | X |
| differential ²⁴ | | | | | | | | | | | | | |
| Serum chemistry ²⁵ | X | X | X²⁵ | X²⁵ | X²⁵ | X²⁵ | X²⁵ | X²⁵ | X²⁵ | X²⁵ | X²⁵ | X | X |
| Coagulation | X | X | X | X | X | X | X | X | X | | X | | |
| parameters ²⁶ | | | | | | | | | | | | | |
| Viral serology ²⁷ | X | | | | | | | | | | | | |
| Immunoglobulins ²⁸ | X | | X | | | | | | | | X | X | X |
| Lymphocyte | X | X | X | | X | | | | X | X | X | X | X |
| subsets ²⁹ | | | | | | | | | | | | | |
| Ferritin, CRP, | X | | X | X | X | X | X | X | X | X | X | | |
| Triglycerides | | | | | | | | | | | | | |

Biomarkers (Blood, Central)

| | | | | | | | | | | | | | |
|---|---|---|---|---|---|---|---|---|---|---|---|---|---|
| CTX130 levels ³⁰ | X | | X³⁰ | X | X | X | X | X | X | X | X | X | X |
| | | | pre/ | | | | | | | | | | |
| | | | post | | | | | | | | | | |
| Cytokines ³¹ | X | | X | X | X | X | X | X | X | X | X | X | X |
| BSAP, PINP ³² | X | | X | | | | X | | X | X | X | | X |
| Anti-Cas9 Ab | X | | | | | | | | | | X | | X |
| Anti-CTX130 Ab | X | | | | | | | | | | X | | X |
| Immunophenotype | X | | X³⁰ | | X | X | X | | X | X | X | X | X |
| | | | pre/ | | | | | | | | | | |
| | | | post | | | | | | | | | | |
| Cell-free DNA | X | | | | | | | | | | X | X | X |
| Exploratory | X | X³⁴ | X | X | X | X | X | X | X | X | X | X | X |
| biomarkers | | | | | | | | | | | | | |

| | Assessment | | | | | | | | |
|---|---|---|---|---|---|---|---|---|---|
| | Follow-Up | | | | | | | | |
| | Day | | | | | | | | |
| | M4 | M5 | M6 | M9 | M12 | M15 | M18 | M21 | M24 |
| | (D112) ± | (D140) ± | (D168) ± | (D252) ± | (D336) ± | (D420) ± | (D504) ± | (D588) ± | (D672) ± |
| | 7 d | 7 d | 7 d | 14 d | 14 d | 14 d | 14 d | 14 d | 14 d |
| Eligibility | | | | | | | | | |
| confirmation ⁵ | | | | | | | | | |
| informed consent | | | | | | | | | |
| Medical history ⁶ | | | | | | | | | |
| Physical exam ⁷ | X | X | X | X | X | X | X | X | X |
| Vital signs ⁸ | X | X | X | X | X | X | X | X | X |
| Height, weight ⁹ | | | | | | | | | |
| Pregnancy test ¹⁰ | | | | | | | | | |
| ECOG status | | | | | | | | | |
| Echocardiogram | | | | | | | | | |
| 12-lead ECG ¹¹ | | | | | | | | | |
| ICE assessment ¹² | | | | | | | | | |
| PRO ¹³ | | | X | | X | | | | X |
| Con meds ¹⁴ | Continuous | | | | | | | | |
| AEs ¹⁵ | Continuous | | | | | | | | |
| Hospital utilization | | | | | | | | | |

B and T Cell Lymphoma Disease/Response Assessments

| | | | | | | | |
|---|---|---|---|---|---|---|---|
| Whole body PET/CT | X | X | X | | X | | X |
| scan ¹⁶ | | | | | | | |
| Brain MRI ¹⁶ | | | | | | | |
| Cutaneous | X | X | X | | X | | X |
| assessment | | | | | | | |
| for all T-cell | | | | | | | |
| lymphomas ¹⁷ | | | | | | | |
| Tumor biopsy ¹⁸ | | | | | | | |
| Peripheral blood | X | X | X | | X | | X |
| assessment ¹⁹ | | | | | | | |
| BM aspirate/ | | | | | | | |
| biopsy ²⁰ | | | | | | | |
| MRD for DLBCL ²¹ | X | X | X | X | X | X | X |
| Disease response | X | X | X | | X | | X |
| assessment for | | | | | | | |
| ATLL ²² | | | | | | | |

Laboratory Assessments (Local) ⁹

| | | | | | | | | | |
|---|---|---|---|---|---|---|---|---|---|
| CD70 expression ²³ | | | | | | | | | |
| CBC w/ | X | X | X | X | X | X | X | X | X |
| differential ²⁴ | | | | | | | | | |
| Serum chemistry ²⁵ | X | X | X | X | X | X | X | X | X |
| Coagulation | | | | | | | | | |
| parameters ²⁶ | | | | | | | | | |
| Viral serology ²⁷ | | | | | | | | | |
| Immunoglobulins ²⁸ | X | X | X | X | X | X | X | X | X |
| Lymphocyte | X | X | X | X | X | X | X | X | X |
| subsets ²⁹ | | | | | | | | | |
| Ferritin, CRP, | X | X | | | | | | | |
| Triglycerides | | | | | | | | | |

Biomarkers (Blood, Central)

| | | | | | | | | | |
|---|---|---|---|---|---|---|---|---|---|
| CTX130 levels ³⁰ | X | X | X | X | X | X | X | X | X |
| Cytokines ³¹ | | | X | | | | | | |
| BSAP, PINP ³² | | | X | | X | | | | |
| Anti-Cas9 Ab | | | X | | X | | | | X |
| Anti-CTX130 Ab | | | X | | X | | | | X |
| Immunophenotype | X | X | X | X | X | X | X | X | X |
| Cell-free DNA | X | X | X | X | X | | X | X | X |
| Exploratory | X | X | X | X | X | X | X | X | X |
| biomarkers | | | | | | | | | |

Ab: antibody; AE: adverse event; AESI: adverse event of special interest; ALT: alanine aminotransferase; AST: aspartate aminotransferase; ATLL: adult T cell leukemia/lymphoma; BM: bone marrow; BSAP: bone-specific alkaline phosphatase; BUN: blood urea nitrogen; Cas9: CRISPR-associated protein 9; CBC: complete blood count; chemo: chemotherapy; CMV: cytomegalovirus; CNS: central nervous system; con meds: concomitant medications; CR: complete response; CRISPR: clustered regularly interspaced short palindromic repeats; CRP: C-reactive protein; CRS: cytokine release syndrome; CT: computed tomography; D or d: day; DLQI: Dermatology Life Quality Index; DNA: deoxyribonucleic acid; ECG: electrocardiogram; ECOG: Eastern Cooperative Oncology Group; eGFR: estimated glomerular filtration rate; EORTC QLQ-C30: European Organisation for Research and Treatment of Cancer QLQ-C30 and questionnaire; FACT-G: Functional Assessment of Cancer Therapy-General; FDG: fluorodeoxyglucose; GvHD: graft versus host disease; HBcAb: hepatitis B core antibody; HBsAb: hepatitis B surface antibody; HBsAg: hepatitis B surface antigen; HBV: hepatitis B virus; HCV: hepatitis C virus; HIV-1/-2: human immunodeficiency virus type 1 or 2; ICE: immune effector cell-associated encephalopathy; Ig: immunoglobulin; INR: international normalized ratio; LD: lymphodepleting; LDH: lactate dehydrogenase; M: month; MF: mycosis fungoides; MRD: minimal residual disease; MRI: magnetic resonance imaging; NK: natural killer; PET: positron emission tomography; PINP: procollagen type I N propeptide; PRO: patient-reported outcomes; PT: prothrombin time; PTT: partial prothrombin time; RNA: ribonucleic acid; SGOT: serum glutamic oxaloacetic transaminase; SGPT: serum glutamic pyruvic transaminase; SS: Sézary syndrome; TBNK: T, B, and NK cells.
¹ Screening assessments to be completed within 14 days of informed consent. Subjects are allowed a one-time rescreening, which may take place within 3 months of initial consent.
² Subjects should start LD chemotherapy within 7 days of study enrollment. Physical exam, weight, and coagulation laboratories are performed prior to first dose of LD chemotherapy. Vital signs, CBC with differential, serum chemistry, and AEs/concomitant medications should be assessed and recorded daily (i.e., 3 times) during LD chemotherapy.
³ CTX130 is administered 48 hours to 7 days after completion of LD chemotherapy.
⁴All baseline assessments on Day 1 are to be performed prior to CTX130 infusion unless otherwise specified; refer to the laboratory manual for details.
⁵ Eligibility should be confirmed each time screening is completed. Eligibility should also be confirmed on the first day of LD chemotherapy, and on day of CTX130 infusion. Eligibility should be confirmed after all assessment for that day are completed and before dosing.
⁶ Includes complete surgical and cardiac history.
⁷ Includes assessment for signs and symptoms of GvHD: skin, oral mucosa, sclera, hands, and feet.
⁸ Includes blood pressure, heart rate, respiratory rate, pulse oximetry, and temperature.
⁹ Height at screening only.
¹⁰ For female subjects of childbearing potential. Serum pregnancy test required at screening. Serum pregnancy test within 72 hours of start of LD chemotherapy, Day 28, M2, and M3. are assessed at a local laboratory.
¹¹ Prior to LD chemotherapy and prior to CTX130 infusion.
¹² On Day 1, prior to CTX130 administration. If CNS symptoms persist, ICE assessment should continue to be performed approximately every 2 days until symptom resolution to Grade 1 or baseline.
¹³ EORTC QLQ-30, EQ-5D-5L questionnaires, FACT-G, Skindex-29 questionnaire for SS and MF, and DLQI questionnaire for SS and MF. PRO should be completed at screening, pre dose on Day 1, Day 7, Day 14, Day 21, Day 28, at Month 3 visit after dosing, and then as specified in the schedule of assessment.
¹⁴ All concomitant medications are collected up to 3 months post-CTX130 infusion. Afterwards, only select concomitant medications are collected as described herein.
¹⁵ AE collection periods are described herein. If a subject begins new anticancer therapy, only AESIs that are possibly related or related to CTX130 should be reported.
¹⁶ Whole body (including neck) PET/CT and MRI brain scan to be performed at screening (i.e., within 28 days prior to CTX130 infusion). Non FDG-avid lymphomas may be followed post baseline by CT as clinically indicated. Whole body (including neck if involved at baseline) PET/CT or CT, as clinically indicated, to be performed upon suspected CR. Postinfusion scans are conducted per the schedule of assessments, per the protocol-defined response criteria and as clinically indicated for all baseline FDG-avid lymphomas. MRI with contrast may be used for the CT portion when CT is clinically contraindicated or as required by local regulation. If PET cannot be performed with diagnostic quality CT, a separate diagnostic CT must be performed. PET/CT is evaluated locally and centrally.
¹⁷Cutaneous assessment to be evaluated locally, but may also be evaluated centrally if indicated (i.e., skin punch biopsy). Skin photographs and mSWAT to be performed post LD chemotherapy Day 3 and prior to CTX130 infusion.
¹⁸ Biopsy (including skin punch biopsy) to be performed at screening if postprogression biopsy tissue is not available/acceptable (Section 7.2.12.1), Day 7 + 2 days, and Day 28 ± 2 days after the dose of CTX130. Tumor biopsy to be evaluated locally and centrally.
¹⁹ Perform peripheral blood assessment per institutional guidelines.
²⁰Bone marrow biopsy and aspirate is collected for all subjects at screening. If a subject is negative for BM infiltration at screening, there is only be a BM biopsy and aspirate collection at Day 28. Otherwise, there are additional BM biopsies and aspirate collections to confirm CR for a subject positive for BM infiltration at screening. BM aspirate/biopsy to be evaluated locally and centrally. Samples from BM aspirate after CTX130 infusion should be sent for CTX130 PK and exploratory biomarkers.
²¹ Minimal residual disease assessment based on peripheral blood. MRD to be evaluated locally and centrally.
²² ATLL-specific biomarkers to be evaluated locally. Changes in peripheral blood levels of ATLL cells as monitored by immunophenotyping based on markers such as CD3, CD4, CD7, CD8, CD25, CD52, and human T cell leukemia virus type 1 (HTLV-1) proviral load.
²³ Subjects must have CD70-expressing tumors (see Section 3.1, Inclusion Criterion #5 for additional details). Tissue may be submitted and tested at any time prior to or during the 14-day window for screening, provided the subject has signed an appropriate consent.
²⁴ Hematocrit, hemoglobin, red blood cell count, white blood cell count, neutrophils, lymphocytes, monocytes, basophils, eosinophils, platelet count, absolute neutrophil count.
²⁵Serum chemistries to include ALT (SGPT), AST (SGOT), bilirubin (total and direct), albumin, alkaline phosphatase, bicarbonate, BUN, calcium, chloride, creatinine, eGFR, glucose, LDH, magnesium, phosphorus, potassium, sodium, total protein, CRP, uric acid (up to Day 28). Creatinine is to be assessed more frequently between Days 1 and 28 to monitor for acute renal tubular damage: daily on Days 1-7, every other day between Days 8-14, and twice weekly until Day 28. If acute renal tubular damage is suspected, additional tests should be conducted including urine sediment analysis and fractional excretion of sodium in urine, and consultation by a nephrologist should be initiated.
²⁶ Include PT, PTT, fibrinogen, INR, and d-dimer.
²⁷ Viral serologies for HIV-1, HIV-2, HBV (HBsAg, HBsAb, HBcAb), HCV (HCV antibody and RNA), and CMV at screening; however, historical results obtained within 60 days of enrollment may be used to determine eligibility.
²⁸ Include IgA, IgG, IgM.
²⁹ Lymphocyte subset assessment at screening, before start of first day of LD chemo, before CTX130 infusion, then all listed time points are assessed at local laboratory. To include 6-color TBNK panel, or equivalent for T, B, and NK cells.
³⁰For CTX130 levels and immunophenotype assessments 2 samples should be collected on Day 1: one pre-CTX130 infusion and one 20 minutes (±5 minutes) after the end of CTX130 infusion. For CTX130 level assessment: if CRS occurs, samples for assessment of CTX130 levels are collected every 48 hours between scheduled visits until CRS resolves. In addition to time points listed, samples for analysis of CTX130 levels should be sent to the central laboratory from any unscheduled collection of blood, BM aspirate, or tissue biopsy performed following CTX130 infusion.
³¹ Additional cytokine samples should be collected daily for the duration of CRS.
³² Samples are to be collected at the same time of day (±2 hours) on the specified collection days.
³³ Samples for exploratory biomarkers should be sent from any LP or BM biopsy performed following CTX130 infusion. If CRS occurs, samples for assessment of exploratory biomarkers are collected every 48 hours between scheduled visits until CRS resolves.
³⁴Prior to first day of LD chemotherapy only.

TABLE 16
Schedule of Assessments: Months 30-60.

| | Assessments | | | | | | | |
|---|---|---|---|---|---|---|---|---|
| | Month 30 | Month 36 | Month 42 | Month 48 | Month 54 | Month 60 | Progressive | Secondary |
| | (±21 days) | (±21 days) | (±21 days) | (±21 days) | (±21 days) | (±21 days) | Disease ¹ | Follow-Up ² |
| Physical exam | X | X | X | X | X | X | X | X |
| Vital signs ³ | X | X | X | X | X | X | X | X |
| PRO ⁴ | | X | | X | | X | X | |
| Concomitant medications ⁵ | X | X | X | X | X | X | X | X |
| AEs ⁶ | X | X | X | X | X | X | X | X |
| Disease/response assessment ⁷ | X | X | X | X | X | X | X | |

Laboratory Assessments (Blood, Local)

| | | | | | | | | |
|---|---|---|---|---|---|---|---|---|
| CBC with differential ⁸ | X | X | X | X | X | X | X | X |
| Serum chemistry ⁸ | X | X | X | X | X | X | X | X |
| Lymphocyte subsets ⁸ | X | X | X | X | X | X | X | |

Biomarkers (Blood, Central)

| | | | | | | | | |
|---|---|---|---|---|---|---|---|---|
| CTX130 levels ⁹ | X | X | X | X | X | X | X | X |
| Anti-Cas9 Ab | | X | | X | | X | X | |
| Anti-CTX130 Ab | | X | | X | | X | X | |
| Cell-free DNA | | | | | | | X | |
| Exploratory biomarkers | X | X | X | X | X | X | X | X |

Ab: antibody; AE: adverse event; AESI: adverse event of special interest; AITL: angioimmunoblastic T cell lymphoma; ALCL: anaplastic large cell lymphoma; ATLL: adult T cell leukemia/lymphoma; BM: bone marrow; Cas9: CRISPR-associated protein 9; CBC: complete blood count; CRISPR: clustered regularly interspaced short palindromic repeats; CT: computed tomography; DLBCL: diffuse large B cell lymphoma; DLQI: Dermatology Life Quality Index; DNA: deoxyribonucleic acid; EORTC QLQ-C30: European Organisation for Research and Treatment of Cancer QLQ-C30 questionnaire; FACT-G: Functional Assessment of Cancer Therapy-General; ISCL: International Society for Cutaneous Lymphomas; M: month; M-protein: monoclonal protein; MF: mycosis fungoides; PD: progressive disease; PET: positron emission tomography; PRO: patient-reported outcomes; PTCL-NOS: peripheral T cell lymphoma, not otherwise specified; SAE: serious adverse event; SCT: stem cell transplantation; SS: Sézary syndrome.
¹ Subjects with PD are discontinue the normal schedule of assessments, undergo study assessments listed, then secondary follow-up (see footnote 2).
² Subjects who partially withdraw consent discontinue the normal schedule of assessments and undergo these procedures, at minimum: abbreviated physical exam, CBC with differential, serum chemistry, disease assessment/survival status, CTX130 persistence, select concomitant medications/procedures (anticancer therapy, disease-related surgery, SCT), and select AEs (treatment-related AEs and SAEs, new malignancies, new/worsening autoimmune, immune deficiency, or neurological disorders).
³ Includes temperature, blood pressure, heart rate, pulse oximetry, and respiratory rate.
⁴ EORTC QLQ-30, EQ-5D-5L questionnaires, FACT-G, Skindex-29 questionnaire for SS and MF, and DLQI questionnaire for SS and MF.
⁵ Only select concomitant medications are collected as described herein.
⁶ If a subject begins new anticancer therapy, only events defined as AESIs as described herein that are possibly related or related to CTX130 should be reported.
⁷ Disease evaluations are based on assessments in accordance with Lugano response criteria (Cheson et al., (2014) J Clin Oncol 32, 3059-68) for subjects with PTCL-NOS, ALCL, leukemic and lymphomatous ATLL, AITL, and DLBCL, and ISCL response criteria (Olsen et al., (2011) J Clin Oncol 29, 2598-607) for subjects with SS or MF, and include whole body PET/CT, BM aspirate and biopsy, and cutaneous assessment.
⁸ Assessed at local laboratory.
⁹ In addition to time points listed, samples for analysis of CTX130 levels should be sent to the central laboratory from any unscheduled collection of blood, BM aspirate, or tissue biopsy performed following CTX130 infusion.

The DLQI is a 10-question questionnaire used to measure the impact of skin disease on the quality of life. The 10 questions cover the following topics: symptoms, embarrassment, shopping and home care, clothes, social and leisure, sport, work or study, close relationships, sex, and treatment. Each question is scored from 0 to 3, giving a possible score range from 0 (meaning no impact of skin disease on quality of life) to 30 (meaning maximum impact on quality of life) (Finlay and Khan, (1994) Clin Exp Dermatol 19, 210-6).

### 6.4 B Cell and T Cell Lymphoma Disease and Response Assessments

Disease evaluations are based on assessments in accordance with the Lugano response criteria (Cheson et al., (2014) J Clin Oncol 32, 3059-68; see Section 6.10) for subjects with PTCL-NOS, ALCL, leukemic and lymphomatous ATLL, AITL, and DLBCL, and according to ISCL response criteria (Olsen et al., (2011) J Clin Oncol 29, 2598-607; see Section 6.11) for subjects with SS or MF.

Disease assessment in the brain should be performed by MRI to rule out brain involvement in subjects during screening.

Per (Olsen et al., (2011) J Clin Oncol 29, 2598-607), the subjects with SS must have:
Measurable disease per Lugano criteria; meeting the definition for SS with ≥80% of body surface area and blood affection.
Erythroderma defined as erythema covering at least 80% body surface area.
A clonal T cell receptor (TCR) rearrangement in the blood identified by polymerase chain reaction (PCR) or southern blot analysis.
An absolute count of Sézary cells in blood of ≥1,000/μL or 1 of the following 2 criteria:
   Increased CD4+ or CD3+ cells with a CD4 to CD8 ratio of 10 or more.
   Increased CD4+ cells with an abnormal phenotype (such as a CD4+CD7− ratio≥40% or a CD4+CD26− ratio≥30%).

For efficacy analyses disease outcome is graded using the Lugano response criteria for the following tumor subtype as assessed for PET/CT imaging or CT imaging for non FDG (fluorodeoxyglucose)-avid disease:
PTCL-NOS
ALCL
Leukemic and lymphomatous ATLL
AITL
DLBCL

For subjects with ATLL hypercalcemia, flares are not considered PD as long as active disease persists and should be treated symptomatically per institutional guidelines. Changes in peripheral blood levels of ATLL cells are monitored by immunophenotyping based on markers such as CD3, CD4, CD7, CD8, CD25, CD52, and human T cell leukemia virus type 1 (HTLV-1) proviral load is to be an exploratory endpoint.

Increased lymphocytosis in the setting of a decrease in lymph node measurement is not considered PD, and response designation should depend on lymph nodes and extranodal disease measurement.

Disease measurement for cutaneous lesions in non CTCLs should follow the guidelines for response assessment of cutaneous lesions as described herein ISCL response criteria are used for subjects with SS or MF as assessed for CT (or if indicated PET/CT) imaging. Erythrodermic flare is not considered disease progression during the first 2 months.

T cell lymphoma disease and response evaluation should be conducted per the schedule in Table 15 and Table 16, and include the assessments described below. All response categories (including progression) require 2 consecutive assessments made at least 1 week apart at any time before the institution of any new therapy.

### 6.5 Pre-CTX130 Biopsy

Histopathological diagnosis of T cell lymphoma subtype is based on local and central laboratory assessment.

Subjects are required to undergo tumor biopsy at screening or, if a biopsy was performed within 3 months prior to enrollment and after the last systemic or targeted therapy, archival tissue may be provided. If archival tissue is of insufficient volume or quality to fulfill central laboratory requirements, a biopsy must be performed during screening. Bone biopsies and other decalcified tissues are not acceptable due to interference with downstream assays. Portions of the tissue biopsy are submitted to a central laboratory for analysis.

Archival tumor tissue samples may be analyzed for tumor intrinsic and TME-specific biomarkers including analysis of DNA, RNA, protein, and metabolites.

### 6.6 Whole Body PET/CT Radiographic Disease Assessment

Whole body (including neck) positron emission tomography (PET)/CT and MRI brain scan to be performed at screening (i.e., within 28 days prior to CTX130 infusion) and upon suspected CR. Postinfusion scans are conducted per the schedule of assessments in Table 15 and Table 16, per the protocol-defined response criteria (see Section 6.10 and Section 6.11), and as clinically indicated for all baseline FDG-avid lymphomas. PET/CT non FDG-avid disease can be followed by CT.

MRI with contrast may be used for the CT portion when CT is clinically contraindicated or as required by local regulation. If PET cannot be performed with diagnostic quality CT, a separate diagnostic CT must be performed.

Whenever possible, the imaging modalities, machines, and scanning parameters used for radiographic disease assessment should be kept consistent during the study. For efficacy analyses, radiographic disease assessments are performed in accordance with protocol-defined response criteria.

### 6.7 Cutaneous Assessment

Cutaneous assessment is performed as specified in Table 15 and Table 16. Initial cutaneous disease assessment should be performed following the third administration of LD chemotherapy and prior to CTX130 infusion. The prognosis of MF and SS depends on the type and extent of skin lesions and extracutaneous disease (Olsen et al., (2011) J Clin Oncol 29, 2598-607). The recommendations based on the consensus guidelines (ISCL, the United States Cutaneous Lymphoma Consortium USCLC]); the Cutaneous Lymphoma Task Force of the EORTC including a scoring system for assessing tumor burden in skin, lymph nodes, blood, and viscera; the definition of response in skin, nodes, blood, and viscera; and a composite global response score are presented in Section 6.11. Response assessment should be support by photographic documentation of representative areas.

### 6.8 Bone Marrow Biopsy and Aspirate

Bone marrow biopsy and aspirate collection at screening are performed for all subjects. If a subject is negative for BM infiltration at screening, there is only a BM biopsy and aspirate collection at Day 28. Otherwise, there are additional BM biopsies and aspirate collections to confirm CR for a subject positive for BM infiltration at screening. Subjects with history of BM involvement who achieve a CR as determined on PET/CT scan have a BM biopsy to confirm response assessment. If a subject shows signs of relapse, the biopsy should be repeated.

Sample for presence of CTX130 (detected via PCR) should be sent for central laboratory evaluation at any point when BM analysis is performed. Samples from BM aspirate after CTX130 infusion should be sent for CTX130 PK and exploratory biomarkers. Standard institutional guidelines for the BM biopsy should be followed. Excess sample (if available) can be stored for exploratory research.

### 6.9 Tumor Biopsy

Subjects are required to undergo tumor biopsy at screening or, if a post-progression biopsy was performed within 3 months prior to enrollment and after the last systemic or targeted therapy, archival tissue may be provided. If archival tissue is of insufficient volume or quality to fulfill central laboratory requirements, a biopsy must be performed during screening as described herein.

Tumor biopsy is performed on Day 7 (+2 days; or as soon as clinically feasible) and Day 28 (±2 days). If a relapse occurs while a subject is on study, every attempt should be made to obtain biopsy of relapse tumor and sent to central laboratory.

Biopsies should come from measurable but non-target lesions. When multiple biopsies are taken, efforts should be made to obtain them from similar tissues. Liver metastases are generally less desirable. Bone biopsies and other decalcified tissues are not acceptable due to interference with downstream assays. This sample is analyzed for presence of CTX130 as well as tumor-intrinsic and TME-specific biomarkers including analysis of DNA, RNA, protein and metabolites.

### 6.10 Lugano Response Criteria, 2014

The following is adapted from Cheson et al., (2014) J Clin Oncol 32, 3059-68.

Diagnosis: A fine-needle aspirate is inadequate for initial diagnosis. An incisional or excisional biopsy is preferred to provide adequate tissue for these examinations. A core-needle biopsy can be considered when excisional biopsy is not possible and to document relapse; however, a non-diagnostic sample must be followed by an incisional or excisional biopsy.

Baseline Site Involvement: Criteria for site involvement are summarized in Table 18.

TABLE 18
Criteria for Involvement of Site.

| Tissue Site | Clinical | FDG Avidity | Test | Positive Finding |
|---|---|---|---|---|
| Lymph | Palpable | FDG-avid | PET-CT | Increased FDG uptake |
| nodes | | histologies | CT | Unexplained node |
| | | Nonavid disease | | enlargement |
| Spleen | Palpable | FDG-avid | PET-CT | Diffuse uptake, solitary |
| | | histologies | CT | mass, miliary lesions, |
| | | Nonavid | | nodules |
| | | disease | | >13 cm in vertical length, |
| | | | | mass, nodules |
| Liver | Palpable | FDG-avid | PET-CT | Diffuse uptake, mass |
| | | histologies | CT | Nodules |
| | | Nonavid disease | | |
| CNS | Signs, | | CT | Mass lesion(s) |
| | symptoms | | MRI | Leptomeningeal |
| | | | CSF | infiltration, mass lesions |
| | | | assessment | Cytology, flow cytometry |
| Other (e.g., | Site | | PET-CT ¹, | Lymphoma involvement |
| skin, lung, GI | dependent | | biopsy | |
| tract, bone, | | | | |
| bone marrow) | | | | |

CSF: cerebrospinal fluid;
CT: computed tomography
FDG: fluorodeoxyglucose;
GI: gastrointestinal;
MRI: magnetic resonance imaging;
PET: positron emission tomography.
¹ PET-CT is adequate for determination of bone marrow involvement and can be considered highly suggestive for involvement of other extralymphatic sites. Biopsy confirmation of those sites can be considered if necessary.

Imaging: Positron emission tomography (PET)-computed tomography (CT) should be used for staging of routinely fluorodeoxyglucose (FDG)-avid histologies. Scan should be reported with visual assessment noting location of foci in nodal and extranodal sites. Images should be scaled to a fixed standardized uptake value and color table; and distinguished from physiological uptake and other patterns of disease according to the distribution and/or CT characteristics.

PET-CT scans should be performed as follows:
As long as possible after the last chemotherapy administration for interim scans
6-8 weeks post chemotherapy at end of treatment ideally (but a minimum of 3 weeks)
≥3 months after radiotherapy

A contrast-enhanced CT scan may be included for a more accurate measurement of nodal size, and to more accurately distinguish bowel from lymphadenopathy; and in the setting of compression/thrombosis of central/mediastinal vessels. Contrast-enhanced CT is also preferred for radiation planning. Variably FDG-avid histologies should be staged with a CT scan.

For subjects staged with CT, disease should be evaluated according to Table 19.

TABLE 19
Disease Evaluation for CT-based Staging.

| Measurable | Measureable Extranodal | Non-Measurable |
|---|---|---|
| Nodal Site | Disease Site | Disease Sites |
| LDi >1.5 cm | LDi >1.0 cm | All other disease sites: |
| | | Nodal Extranodal |
| | | Assessable disease |

| | |
|---|---|
| Up to 6 measurable nodal/extranodal sites: | Examples: |
| Largest target nodes, nodal masses | Skin, GI, bone, spleen, |
| or other lymphomatous lesions | liver, kidneys, effusions |
| Measurable extranodal disease | |
| Measurable in 2 diameters (LDi and SDi) | |
| Represent different body regions/overall disease burden | |
| Include mediastinal and retroperitoneal disease, if involved | |

GI: gastrointestinal;
LDi: longest transverse diameter of a lesion;
SDi: shortest axis perpendicular to LDi.

Tumor Bulk: A single nodal mass, in contrast to multiple smaller nodes, of 10 cm or greater than a third of the transthoracic diameter at any level of thoracic vertebrae as determined by CT is the definition of bulky disease for Hodgkin lymphoma (HL). A chest x-ray is not required to determine bulk. For HL and non-Hodgkin lymphoma (NHL) the longest measurement by CT scan should be recorded.

Measurements of total tumor volume should be explored as potential prognosticators with PET and CT.

Spleen Liver and Bone Marrow Involvement: Splenic and liver involvement are best determined by PET-CT as described in Table 20.

TABLE 20
Spleen and Liver Involvement.

| Spleen | Liver |
|---|---|
| Use single measurement | Liver size by physical examination or CT |
| which correlates well | scan not a reliable measure of hepatic |
| with volume | involvement by lymphoma |
| Most studies use 10-12 cm | Diffusely increased or |
| for vertical length | focal uptake, with or |
| (cranial to caudal) | without focal or |
| Lugano recommendation: | disseminated nodules |
| Splenomegaly >13 cm | support liver involvement |

Bone marrow involvement may be determined as follows:
HL, if PET-CT is performed, bone marrow biopsy (BMB) is not required.
DLBCL, BMB if the PET is negative and identifying a discordant histology is important for subject management.
Other subtypes, ˜2.5 cm unilateral BMB is recommended, along with immunohistochemistry and flow cytometry at screening/baseline.
If uninvolved at baseline, must be normal for CR and evidence of FDG-avid disease in marrow for complete metabolic response (CMR).

Staging System: A modified Ann Arbor staging system should be used for anatomic description of disease extent (Table 21).

TABLE 21
Revised Staging System for Primary Nodal Lymphomas

| Stage | Involvement | Extranodal Status |
|---|---|---|
| Limited | | |
| Stage 1 | One node or group of adjacent nodes | Single extranodal lesion without |
| | | nodal involvement |
| Stage II | Two or more nodal groups on the | Stage I or II by nodal extent with |
| | same side of the diaphragm | limited, contiguous extranodal |
| | | involvement |
| Stage II bulky ¹ | II as above with bulky disease | N/A |
| Advanced | | |
| Stage III | Nodes on both sides of the diaphragm | N/A |
| | Nodes above the diaphragm with | |
| | spleen involvement | |
| Stage IV | Additional noncontiguous extranodal | N/A |
| | involvement | |

NOTE.
Extent of disease is determined by positron emission tomography-computed tomography for avid lymphomas and computed tomography for nonavid histologies. Tonsils, Waldeyer’s ring, and spleen are considered nodal tissue.
¹ Whether Stage II bulky disease is treated as limited or advanced disease may be determined by histology and a number of prognostic factors.

Response Assessment: PET-CT should be used for response assessment in FDG-avid histologies, using the 5-point scale; CT is preferred for low or variable FDG avidity.

Surveillance scans after remission are discouraged, especially for DLBCL and HL, although a repeat study may be considered after an equivocal finding after treatment.

Judicious use of follow-up scans may be considered in indolent lymphomas with residual intra-abdominal or retroperitoneal disease.

Criteria for response are summarized in Table 22.

TABLE 22
Revised Criteria for Response Assessment.

| Response and Site | PET/CT-based Response | CT-based Response |
|---|---|---|
| Complete | Complete metabolic response | Complete radiologic response (all of the |
| | | following) |
| Lymph nodes & extralymphatic sites | Score 1, 2, or 3¹ with or without a residual | Target nodes/nodal masses must regress |
| | mass on 5PS² | to ≤ 1.5 cm in LDi |
| | It is recognized that in Waldeyer’s ring or | No extralymphatic sites of disease |
| | extranodal sites with high physiologic uptake | |
| | or with activation within spleen or marrow | |
| | (e.g., with chemotherapy or myeloid colony- | |
| | stimulating factors), uptake may be greater | |
| | than normal mediastinum and/or liver. In this | |
| | circumstance, complete metabolic response | |
| | may be inferred if uptake at sites of initial | |
| | involvement is no greater than surrounding | |
| | normal tissue even if the tissue has high | |
| | physiologic uptake. | |
| Nonmeasured lesion | Not applicable | Absent |
| Organ enlargement | Not applicable | Regress to normal |
| New lesions | None | None |
| Bone marrow | No evidence of FDG-avid disease in marrow | Normal by morphology; if indeterminate, IHC |
| | | negative |
| Partial | Partial metabolic response | Partial remission (all of the following) |
| Lymph nodes & extralymphatic sites | Score 4 or 5² with reduced uptake compared | ≥50% decrease in SPD of up to 6 target |
| | with baseline and residual mass(es) of any | measurable nodes and extranodal sites |
| | size | When a lesion is too small to measure on CT, |
| | At interim, these findings suggest responding | assign 5 mm × 5 mm as the default value |
| | disease | When no longer visible, 0 × 0 mm |
| | At end of treatment, these findings indicate | For a node > 5 mm × 5 mm, but small |
| | residual disease | |
| Nonmeasured lesion | Not applicable | Absent/normal, regressed, but no increase |
| Organ enlargement | Not applicable | Spleen must have regressed by > 50% in |
| | | length beyond normal |
| New lesions | None | None |
| Bone marrow | Residual uptake higher than uptake in normal | Not applicable |
| | marrow but reduced compared with baseline | |
| | (diffuse uptake compatible with reactive | |
| | changes from chemotherapy allowed). If there | |
| | are persistent focal changes in the marrow in | |
| | the context of a nodal response, consideration | |
| | should be given to further evaluation with | |
| | MRI or biopsy or an interval scan. | |
| No response or stable disease | No metabolic response | Stable disease |
| Target nodes/nodal masses, extranodal | Score 4 or 5 with no significant change in | <50% decrease from baseline in SPD of up to |
| Lesions | FDG uptake from baseline at interim or | 6 dominant, measurable nodes and extranodal |
| | end of treatment | sites; no criteria for progressive disease are |
| | | met |
| Nonmeasured lesion | Not applicable | No increase consistent with progression |
| Organ enlargement | Not applicable | No increase consistent with progression |
| New lesions | None | None |
| Bone marrow | No change from baseline | Not applicable |
| | | Progressive disease requires at least 1 of |
| Progressive disease | Progressive metabolic disease | the following |
| Individual target nodes/nodal masses | Score 4 or 5 with an increase in intensity | PPD progression: |
| Extranodal lesions | of uptake from baseline and/or | An individual node/lesion must be abnormal |
| | New FDG-avid foci consistent with | with: |
| | lymphoma at interim or | LDi > 1.5 cm and |
| | end-of-treatment assessment | Increase by ≥ 50% from PPD nadir and |
| | | An increase in LDi or SDi from nadir |
| | | 0.5 cm for lesions ≤ 2 cm |
| | | 1.0 cm for lesions > 2 cm |
| Response and Site | PET/CT-based Response | CT-based Response |
| | | In the setting of splenomegaly, the splenic |
| | | length must increase by > 50% of the |
| | | extent of its prior increase beyond |
| | | baseline (e.g., a 15-cm spleen must |
| | | increase to > 16 cm). If no prior |
| | | splenomegaly, must increase by at least 2 |
| | | cm from baseline |
| | | New or recurrent splenomegaly |
| Nonmeasured lesion | None | New or clear progression of pre-existing non- |
| | | measured lesions |
| New lesions | New FDG-avid foci consistent with | Regrowth of previously resolved lesions |
| | lymphoma rather than another etiology (e.g., | A new node > 1.5 cm in any axis |
| | infection, inflammation). If uncertain | A new extranodal site > 1.0 cm in any |
| | regarding etiology of new lesions, biopsy or | axis; if < 1.0 cm in any axis, its presence |
| | interval scan may be considered | must be unequivocal and must be |
| | | attributable to lymphoma |
| | | Assessable disease of any size unequivocally |
| | | attributable to lymphoma |
| Bone marrow | New or recurrent FDG-avid foci | New or recurrent involvement |

5PS: 5-point scale;
CT: computed tomography;
FDG: fluorodeoxyglucose;
IHC: immunohistochemistry;
LDi: longest transverse diameter of a lesion;
MRI: magnetic resonance imaging;
PET: positron emission tomography;
PPD: cross product of the LDi and perpendicular diameter;
SDi: shortest axis perpendicular to the LDi;
SPD: sum of the product of the perpendicular diameters for multiple lesions.
¹A score of 3 in many subjects indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to 6 of the largest dominant nodes, nodal masses, and extranodal lesions selected to be clearly measurable in 2 diameters. Nodes should preferably be from disparate regions of the body and should include, where applicable, mediastinal and retroperitoneal areas. Non-nodal lesions include those in solid organs (e.g., liver, spleen, kidneys, lungs), GI involvement, cutaneous lesions, or those noted on palpation. Nonmeasured lesions: Any disease not selected as measured, dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer’s ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
²PET 5-Point Scale: 1, no uptake above background; 2, uptake ≤ mediastinum; 3, uptake ≥ mediastinum but ≤ liver; 4, uptake moderately > liver; 5, uptake markedly higher than liver and/or new lesions; X, new areas of uptake unlikely to be related to lymphoma.

### 6.11 International Society for Cutaneous Lymphoma Response Criteria, 2011

The following is adapted from Olsen et al., (2011) J Clin Oncol. 29, 18:2598-607.

Definitions: Definitions of patch, plaque, and tumor to be used are outlined in Table 23.

TABLE 23
Modified ISCL/EORTC Revisions to the TNMB Classification of MF/SS.

| TNMB | |
|---|---|
| Stages | Description of TNMB |
| Skin ¹ | |
| T₁ | Limited patches, papules, and/or plaques covering < 10% of the skin surface; |
| | may further stratify into T₁ₐ (patch only) v T_{1b} (plaque ± patch) |
| T₂ | Patches, papules, or plaques covering ≥ 10% of the skin surface; may further |
| | stratify into T₂ₐ (patch only) v T_{2b} (plaque ± patch) |
| T₃ | One or more tumors (≥1 cm diameter) |
| T₄ | Confluence of erythema covering ≥ 80% body surface area |
| Node ² | |
| N₀ | No clinically abnormal lymph nodes; biopsy not required |
| N₁ | Clinically abnormal lymph nodes; histopathology Dutch Grade 1 or NCI LN₀₋₂ |
| N₁ₐ | Clone negative |
| N_{1b} | Clone positive |
| N₂ | Clinically abnormal lymph nodes; histopathology Dutch Grade 2 or NCI LN₃ |
| N₂ₐ | Clone negative |
| N_{2b} | Clone positive |
| N₃ | Clinically abnormal lymph nodes; histopathology Dutch Grade 3-4 or NCI LN₄; |
| | clone positive or negative |
| Nₓ | Clinically abnormal lymph nodes without histologic confirmation or inability to |
| | fully characterize the histologic subcategories |
| Visceral | |
| M₀ | No visceral organ involvement |
| M₁ | Visceral involvement (must have pathology confirmation and organ involved |
| | should be specified) |
| Blood | |
| B₀ | Absence of significant blood involvement: ≤5% of peripheral blood |
| | lymphocytes are atypical (Sézary) cells |
| B₀ₐ | Clone negative |
| B_{0b} | Clone positive |
| B₁ | Low blood tumor burden: >5% of peripheral blood lymphocytes are atypical |
| | (Sézary) cells but does not meet the criteria of B₂ |
| B₁ₐ | Clone negative |
| B_{1b} | Clone positive |
| B₂ | High blood tumor burden: ≥1,000/μL Sézary cells with positive clone ³; 1 of the |
| | following can be substituted for Sézary cells: CD4/CD8 ≥ 10, CD4 + CD7 − |
| | cells ≥ 40% or CD4 + CD26 − cells ≥ 30% |

EORTC: European Organisation for Research and Treatment of Cancer;
ISCL: International Society for Cutaneous Lymphomas;
MF: mycosis fungoides;
NCI: National Cancer Institute
SS: Sézary syndrome;
TNMB: tumor-node-metastasis-blood.
¹ Patch = any size lesion without induration or significant elevation above the surrounding uninvolved skin: pokiloderma may be present. Plaque = any size lesion that is elevated or indurated: crusting or poikiloderma may be present. Tumor = any solid or nodular lesion ≥ 1 cm in diameter with evidence of deep infiltration in the skin and/or vertical growth.
² Lymph node classification has been modified from 2007 ISCL/EORTC consensus revisions to include central nodes. Lymph nodes are qualified as abnormal if > 1.5 cm in diameter.
³ The clone in the blood should match that of the skin. The relevance of an isolated clone in the blood or a clone in the blood that does not match the clone in the skin remains to be determined.

Diagnosis: Histopathologic diagnosis should be confirmed in a skin biopsy representative of current disease by a pathologist with expertise in cutaneous lymphoma. For Sézary syndrome (SS; defined as meeting T₄ plus B₂ criteria), where the biopsy of erythrodermic skin may only reveal suggestive but not diagnostic histopathologic features, the diagnosis may be based on either a node biopsy or fulfillment of B₂ criteria including a clone in the blood that matches that of the skin. For early patch stage mycosis fungoides (MF) where the histological diagnosis by light microscopic examination is not confirmed, diagnostic criteria that have been recommended by the ISCL should be used.

Evaluation:
Pretreatment evaluation and scoring of response parameters should be done at baseline (day 1 of treatment), and not at screening.
All responses should be at least 4 weeks in duration.

Skin Assessment, Scoring, and Definition of Response:

The Severity Weighted Assessment Tool (SWAT) or the modified SWAT (mSWAT) should be used for skin scoring.

The definition of response is presented in Table 24.

TABLE 24
Response in Skin.

| Response | Definition |
|---|---|
| Complete response | 100% clearance of skin lesions ¹ |
| Partial response | 50%-99% clearance of skin disease from baseline without new |
| | tumors (T₃) in subjects with T₁, T₂ or T₄ only skin disease |
| Stable disease | <25% increase to < 50% clearance in skin disease from baseline |
| | without new tumors (T₃) in subjects with T₁, T₂, or T₄ only skin |
| | disease |
| Progressive disease ² | ≥25% increase in skin disease from baseline or New tumors (T₃) in |
| | subjects with T₁, T₂ or T₄ only skin disease or Loss of response: in |
| | those with complete or partial response, increase of skin score of |
| | greater than the sum of nadir plus 50% baseline score |
| Relapse | Any disease recurrence in those with complete response |

NOTE.
Based on modified Severity Weighted Assessment Tool score.
¹ A biopsy of normal appearing skin is unnecessary to assign a complete response. However, a skin biopsy should be performed of a representative area of the skin if there is any question of residual disease (persistent erythema or pigmentary change) where otherwise a complete response would exist. If histologic features are suspicious or suggestive of mycosis fungoides/Sézary syndrome (see histologic criteria for early mycosis fungoides), the response should be considered a partial response only.
² Whichever criterion occurs first.

Lymph Node Assessment, Scoring, and Definition of Response:

Peripheral lymph nodes: The full tumor-node-metastasis-blood (TNMB) status of participants should be characterized, and computed tomography (CT) imaging is recommended, with the caveat that considerable inter-observer variability exists. Magnetic resonance imaging (MRI) is an alternative to CT.

Central lymph nodes: If there is evidence of enlarged central nodes (defined as >1.5 cm diameter in the long axis or >1.0 cm diameter in the short axis), and confirmation of involvement with MF/SS by biopsy (i.e., excisional, fine-needle aspirate, or core biopsy), then all central nodes should be tracked thereafter in the same way as peripheral nodes (product of the longest bidimensional measurements of all enlarged nodes)

The definition of response is presented in Table 25.

TABLE 25
Response in Lymph Nodes.

| Response | Definition ¹ |
|---|---|
| Complete response | All lymph nodes are now ≤ 1.5 cm in greatest transverse (long |
| | axis) diameter by method used to assess lymph nodes at baseline |
| | or biopsy negative for lymphoma; in addition, lymph nodes that |
| | were N₃ classification and ≤ 1.5 cm in their long axis and > 1 cm |
| | in their short axis at baseline, must now be ≤ 1 cm in their short |
| | axis or biopsy negative for lymphoma |
| Partial response | Cumulative reduction ≥ 50% of the SPD of each abnormal lymph |
| | node at baseline and no new lymph node > 1.5 cm in the diameter |
| | of the long axis or > 1.0 cm in the diameter of the short axis if the |
| | long axis is 1-1.5 cm diameter |
| Stable disease | Fails to attain the criteria for CR, PR, and PD |
| Progressive disease ² | ≥50% increase in SPD from baseline of lymph nodes or |
| | Any new node > 1.5 cm in the long axis or > 1 cm in the short |
| | axis if 1-1.5 cm in the long axis that is proven to be N3 |
| | histologically or |
| | Loss of response: >50% increase from nadir in SPD of lymph |
| | nodes in those with PR |
| Relapse | Any new lymph node > 1.5 cm in the long axis in those with CR |
| | proven to be N₃ histologically |

CR: complete response;
PD: progressive disease;
PR: partial response;
SPD: sum of the maximum linear dimension (major axis) × longest perpendicular dimension (minor axis).
¹ Peripheral and central lymph nodes.
² Whichever criterion occurs first.

Visceral Disease Assessment, Scoring, and Definition of Response: Biopsy confirmation at baseline is recommended for all forms of visceral disease except for liver and spleen involvement, which may be diagnosed by imaging studies. Of note, bone marrow aspirate/trephine biopsies are not considered obligatory for either evaluation or response assessment. There may be limitations in corroborating a CR in viscera by CT alone, and in those cases, a confirmatory biopsy may be necessary or lacking this, no CR assessment can be made. The definition of response is presented in Table 26.

TABLE 26
Response in Viscera

| Response | Definition |
|---|---|
| Complete response | Liver or spleen or any organ considered involved at baseline |
| | should not be enlarged on physical exam and should be considered |
| | normal by imaging; no nodules should be present on imaging of |
| | liver or spleen; any post-treatment mass must be determined by |
| | biopsy to be negative for lymphoma |
| Partial response | ≥50% regression in any splenic or liver nodules, or in measureable |
| | disease (SPD) in any organs abnormal at baseline; no increase in |
| | size of liver or spleen and no new sites of involvement |
| Stable disease | Fails to attain the criteria for CR, PR, or PD |
| Progressive disease ² | >50% increase in size (SPD) of any organs involved at baseline or |
| | New organ involvement or |
| | Loss of response: >50% increase from nadir in the size (SPD) of |
| | any previous organ involvement in those with PR |
| Relapse | New organ involvement in those with CR |

CR: complete response;
PR: partial response;
SPD: sum of the maximum linear dimension (major axis) × longest perpendicular dimension (minor axis);
SD: stable disease;
PD: progressive disease.
¹ Whichever criterion occurs first.

Blood Assessment, Scoring, and Definition of Response: The absolute number of CD4⁺CD26⁻ cells determined by flow cytometry is the most reasonable, quantifiable measure of potential blood involvement in MF/SS. In CD26⁺ subjects, CD4⁺CD7⁻ T cells would be an alternate population to monitor.

Based on an upper limit of normal value of 1,600/μL for CD4 cells in the blood, an absolute count of lower than 250/μL CD4⁺/CD26⁻ or CD4⁺CD7⁻ cells would appear to be a normal value for these CD4 subsets and could also be used to define the absence of or normalization of blood involvement (Bo). Alternately, an absolute Sézary cell count is an optional method when good quality smears are interpreted by a single qualified reader with lower than 250/μL and higher than 1,000/μL of Sézary cells being reasonable determinants of B₀ and B₂.

The definition of response is presented in Table 27.

TABLE 27
Response in Blood ¹

| Response | Definition |
|---|---|
| Complete response ² | B₀ |
| Partial response ³ | >50% decrease in quantitative measurements of blood tumor burden |
| | from baseline in those with high tumor burden at baseline (B₂) |
| Stable disease | Fails to attain criteria for CR, PR, or PD |
| Progressive disease ⁴ | B₀ to B₂ or > 50% increase from baseline and at least 5,000 |
| | neoplastic cells/μL or |
| | Loss of response: in those with PR who were originally B2 at |
| | baseline, >50% increase from nadir and at least 5,000 neoplastic |
| | cells/μL |
| Relapse | Increase of neoplastic blood lymphocytes to ≥ B₁ in those with CR |
| | CR: complete |

CR: complete response;
PR: partial response;
SD: stable disease;
PD: progressive disease.
¹ As determined by absolute numbers of neoplastic cells/μL.
² If a bone marrow biopsy was performed at baseline and determined to unequivocally be indicative of lymphomatous involvement, then to confirm a global CR where blood assessment now meets criteria for B₀, a repeat bone marrow biopsy must show no residual disease or the response should be considered a PR only.
³ There is no PR in those with B1 disease at baseline as the difference within the range of neoplastic cells that define B₁ is not considered significant and should not affect determination of global objective response.
⁴ Whichever occurs first.

Global Response Score Definition: Consensus global response score for MF/SS is presented in Table 28.

TABLE 28
Global Response Score.

| | | | | | |
|---|---|---|---|---|---|
| Global Score¹ | Definition | Skin | Nodes | Blood | Viscera |

| | | | |
|---|---|---|---|
| CR | Complete disappearance of | CR | All categories have CR/NI |
| | all clinical evidence of | | |
| | disease | | |
| PR | Regression of measurable | CR | All categories do not have a CR/NI |
| | disease | | and no category has a PD |
| | | PR | No category has a PD and if any |
| | | | category involved at baseline, at |
| | | | least 1 has a CR or PR |
| SD | Failure to attain CR, PR, or | PR | No category has a PD and if any |
| | PD representative of all | | category involved at baseline, no |
| | disease | | CR or PR in any |
| PD | Progressive disease | SD | CR/NI, PR, SD in any category and |
| | | | no category has a PD in any |
| | | | category |
| Relapse | Recurrence disease in prior | | Relapse in any category |
| | CR | | |

CR: complete response;
NI: noninvolved;
PR: partial response;
PD: progressive disease; SD: stable disease.
¹It is recommended that not only the proportion of subjects who achieve a response or an unfavorable outcome be calculated but a life table account for the length of the interval during which each subject is under observation also be generated.

### 7. Treatment

### 7.1. Lymphodepleting Chemotherapy

All subjects receive LD chemotherapy prior to infusion of CTX130. LD chemotherapy consists of:
Fludarabine 30 mg/m² IV daily for 3 doses, AND
Cyclophosphamide 500 mg/m² IV daily for 3 doses.

Adult subjects with moderate impairment of renal function (CrCl 50-70 mL/min/1.73 m²) should receive a reduced dose of fludarabine by at least 20% or in accordance with local prescribing information.

Both agents are started on the same day and administered for 3 consecutive days. Subjects should start LD chemotherapy within 7 days of study enrollment.

Reference the current full prescribing information for fludarabine and cyclophosphamide for guidance regarding the storage, preparation, administration, supportive care instructions, and toxicity management associated with LD chemotherapy.

LD chemotherapy can be delayed if any of the following signs or symptoms are present:
Change in performance status to ECOG>1.
Significant worsening of clinical status that increases the potential risk of AEs associated with LD chemotherapy, e.g.:
   Clinically significant worsening of any cytopenia,
   Clinically significant increase of transaminase levels (e.g., >3×ULN),
   Clinically significant increase of total bilirubin (e.g., >2×ULN), or
   Clinically significant increase in serum creatinine.
Requirement for supplemental oxygen to maintain a saturation level of >92%.
New uncontrolled cardiac arrhythmia.
Hypotension requiring vasopressor support.
Active infection: Positive blood cultures for bacteria, fungus, or virus not responding to treatment.
Any acute neurological toxicity (e.g., ≥2 acute neurological toxicity).

### 7.2. Administration of CTX130

CTX130 infusion is to be delayed if any of the following signs or symptoms are present:
Change in performance status to ECOG>1.
New active uncontrolled infection.
Significant worsening of clinical status, increases the potential risk of AEs associated with allogenic CAR T cell infusion, e.g.:
   Clinically significant increase of transaminase levels (e.g., >3×ULN),
   Clinically significant increase of total bilirubin (e.g., >2×ULN), or
   Clinically significant increase in serum creatinine.
Any acute neurological toxicity (e.g., ≥2 acute neurological toxicity).

CTX130 is administered at least 48 hours (but no more than 7 days) after the completion of LD chemotherapy.

Given the potential clinical benefit that can be derived from repeat dosing, the current study allows repeat dosing of CTX130 for up to two times at Month 2 after CTX130 infusion to have a maximum of 3 doses in the study.

Repeat dosing, at Month 3 after CTX130 infusion, may occur in the following scenarios:
Progressive Disease—At Month 2 post infusion of CTX130, if new lesions or growth>20% are observed (Lugano and ISCL response criteria), then consider redosing if the progression event does not constitute a clinically threatening scenario.
Stable Disease or Partial Response—re-dose; redosing occurs if complete remission has not been achieved by Month 3.
Complete Remission—no redosing.

In some instances, no more than 2 times redosing of subjects with CTX130 cells may be allowed. To be considered for redosing, subjects must have either 1) achieved a partial response (PR) or complete response (CR) after initial or second CTX130 infusion and subsequently progressed within 2 years of last dose, or 2) stable disease (SD) at the Month 1 study visit after the most recent CTX130 infusion (redosing decisions will be based upon local CT scan/assessment). The earliest time at which a subject could be redosed is 6 weeks after the initial or second CTX130 infusion.

In order to be considered for the redosing, subjects need to meet the following criteria:
No DLT during dose-escalation (if applicable).
No Grade≥3 (e.g., 4) CRS that didn't resolve to Grade≤2 (e.g., 2) within 72 hours following the CTX130 infusion.
No Grade>1 GVHD following CTX130 infusion.
No Grade≥2 (e.g., ≥3) ICANS following CTX130 infusion.
Meeting criteria for LD chemotherapy and CTX130 infusion (e.g., hemodynamically stable, no active infections).
Meeting all end organ criteria (e.g., liver, renal, cardiac, pulmonary, neurological) as in inclusion/exclusion criteria.

Prior to each dosing event, subjects receive another dose of LD chemotherapy. In Parts A and B a subject may be redosed up to two times at Month 3 after CTX130 infusion, to have a maximum of 3 doses in the study. In Part A, intrasubject dose escalation is allowed, if the subject did not experience a DLT at the previous dose level and no DLT was observed at the next higher dose level during the DLT evaluation period. Intrasubject dose escalation is allowed only once to the next higher dose level, if the dose is cleared, and if the subject continues to have benefit and does not violate any of the redosing criteria.

### 7.3. CTX130 Post-Infusion Monitoring

Following CTX130 infusion, subjects' vitals should be monitored every 30 minutes for 2 hours after infusion or until resolution of any potential clinical symptoms.

Subjects in Part A are hospitalized for a minimum of 7 days after CTX130 infusion. In both Parts A and B, subjects must remain in proximity of the investigative site (i.e., 1-hour transit time) for at least 28 days after CTX130 infusion. Management of acute CTX130-related toxicities should occur ONLY at the study site.

Subjects are monitored for signs of cytokine release syndrome (CRS), tumor lysis syndrome (TLS), neurotoxicity, graft versus host disease (GvHD), and other adverse events (AEs) according to the schedule of assessments (Table 15 and Table 16). Guidelines for the management of CAR T cell-related toxicities are described in Section 8. Subjects should remain hospitalized until CTX130-related nonhematologic toxicities (e.g., fever, hypotension, hypoxia, ongoing neurological toxicity) return to Grade 1. Subjects may remain hospitalized for longer periods if considered necessary by medical administrators.

### 7.4. Prior and Concomitant Medications

7.4.1 Allowed Medications Necessary supportive measures for optimal medical care are given throughout the study, including IV antibiotics to treat infections, growth factors, blood components, etc., except for prohibited medications described herein.

Medications to inhibit bone absorption such as biposphonates or RANKL inhibitor are allowed per medical administrator discretion for symptomatic therapy including hypercalcemia.

All concurrent therapies, including prescription and nonprescription medication, and medical procedures must be recorded from the date of signed informed consent through 3 months after CTX130 infusion. Beginning 3 months post-CTX130 infusion, only the following selected concomitant medications are collected: vaccinations, anticancer treatments (e.g., chemotherapy, radiation, immunotherapy), immunosuppressants (including steroids), and any investigational agents.

### 7.4.2 Prohibited Medications

The following medications are prohibited during certain periods of the study as specified below:

Prohibited within 28 Days Before and 3 Months after CTX130 Infusion
Live vaccines.
Herbal medicine as part of traditional Chinese medicine or non-over-the-counter herbal remedies.

Prohibited Throughout the Study Until the Start of New Anticancer Therapy
Any immunosuppressive therapy unless recommended to treat CRS or ICANS or if previously discussed with and approved by the medical administrator.
Corticosteroid therapy at a pharmacologic dose (>10 mg/day of prednisone or equivalent doses of other corticosteroids) and other immunosuppressive drugs should be avoided after CTX130 administration unless medically indicated to treat new toxicity or as part of management of CRS or neurotoxicity associated with CTX130.
Any anticancer therapy (e.g., chemotherapy, immunotherapy, targeted therapy, radiation, or other investigational agents) other than LD chemotherapy prior to disease progression. Palliative radiation therapy for symptom management is permitted depending on extent, dose, and site(s). site(s), dose, and extent should be defined and discussed with the medical administrator for determination.

Prohibited within the First Month after CTX130 Infusion
Granulocyte-macrophage colony-stimulating factor (GM-CSF) following CTX130 infusion due to the potential to worsen symptoms of CRS; Care should be taken with administration of granulocyte colony-stimulating factor (G-CSF) following CTX130.
During the DLT evaluation period (28 days), self-medication by the subject with antipyretics (e.g., acetaminophen, aspirin).

Prohibited 3 Months Prior and During the Treatment with CTX130, and Up to 6 Months after CTX130 Infusion
CCR-4-directed antibodies like mogamulizumab due to the increased risk of GvHD.

### 8. Toxicity Management

### 8.1 General Guidance

Subjects must be closely monitored for at least 28 days after CTX130 infusion. Significant toxicities have been reported with autologous CAR T cell therapies and proactively monitor and treat all adverse events (AEs) are required in accordance with protocol guidance.

The following general recommendations are provided based on prior experience with CD70-directed autologous CAR T cell therapies:
Fever is the most common early manifestation of cytokine release syndrome (CRS); however, subjects may also experience weakness, hypotension, or confusion as first presentation.
Diagnosis of CRS should be based on clinical symptoms and NOT laboratory values.
In subjects who do not respond to CRS-specific management, always consider sepsis and resistant infections. Subjects should be continually evaluated for resistant or emergent bacterial infections, as well as fungal or viral infections.
CRS, HLH, and TLS may occur at the same time following CAR T cell infusion. Subjects should be consistently monitored for signs and symptoms of all the conditions and managed appropriately.
ICANS may occur at the time of CRS, during CRS resolution, or following resolution of CRS. Grading and management of ICANS are performed separately from CRS.
Tocilizumab must be administered within 2 hours from the time of order.

The safety profile of CTX130 is continually assessed throughout the study.

### 8.2 Toxicity-Specific Guidance

### 8.2.1 Infusion Reactions

Infusion-related reactions have been reported in autologous CAR T cell trials, including transient fever, chills, and/or nausea most commonly occurring within 12 hours after administration. Acetaminophen (paracetamol) and diphenhydramine hydrochloride (or another H1-antihistamine) may be repeated every 6 hours after CTX130 infusion, as needed, if an infusion reaction occurs. Nonsteroidal anti-inflammatory medications may be prescribed as needed if the subject continues to have fever not relieved by acetaminophen. Systemic steroids should NOT be administered except in cases of life-threatening emergency, as this intervention may have a deleterious effect on CAR T cells.

### 8.2.2 Infection Prophylaxis and Febrile Reaction

Infection prophylaxis should be managed according to the institutional standard of care for patients with T cell or B cell malignancies. In the event of febrile reaction, an evaluation for infection should be initiated and the subject managed appropriately with antibiotics, fluids, and other supportive care as medically indicated and determined by the treating physician. Viral and fungal infections should be considered throughout a subject's medical management if fever persists. If a subject develops sepsis or systemic bacteremia following CTX130 infusion, appropriate cultures and medical management should be initiated. Additionally, consideration of CRS should be given in any instances of fever following CTX130 infusion within 28 days post infusion.

Viral encephalitis (e.g., human herpes virus [HHV]-6 encephalitis) must be considered in the differential diagnosis for subjects who experience neurocognitive symptoms after receiving CTX130. A lumbar puncture (LP) is required for any Grade 3 or higher neurocognitive toxicity and is strongly recommended for Grade 1 and Grade 2 events. Whenever a lumbar puncture is performed, an infectious disease panel will review data from the following assessments (at a minimum): quantitative testing for HSV 1&2, Enterovirus, Human Parechovirus, VZV, CMV, and HHV-6. Lumbar puncture must be performed within 48 hours of symptom onset and results from the infectious disease panel must be available within 4 days of the LP in order to appropriately manage the subject.

### 8.2.3 Tumor Lysis Syndrome (TLS)

Subjects receiving CAR T cell therapy may be at increased risk of TLS. Subjects should be closely monitored for TLS via laboratory assessments and symptoms from the start of LD chemotherapy until 28 days following CTX130 infusion. Subjects at increased risk of TLS should receive prophylactic allopurinol (or a nonallopurinol alternative such as febuxostat) and/or rasburicase and increased oral/IV hydration during screening and before initiation of LD chemotherapy. Prophylaxis can be stopped after 28 days following CTX130 infusion or once the risk of TLS passes.

Sites should monitor and treat TLS as per their institutional standard of care, or according to published guidelines (Cairo and Bishop, (2004) Br J Haematol, 127, 3-11). TLS management, including administration of rasburicase, should be instituted promptly when clinically indicated.

### 8.2.4 Cytokine Release Syndrome (CRS)

CRS is a toxicity associated with immune therapies, including CAR T cells, resulting from a release of cytokines, in particular IL-6 and IL-1 (Norelli et al., 2018). CRS is due to hyperactivation of the immune system in response to CAR engagement of the target antigen, resulting in multicytokine elevation from rapid T cell stimulation and proliferation (Frey et al., 2014; Maude et al., 2014a).

The clinical presentation of CRS may be mild and be limited to elevated temperatures or can involve one or multiple organ systems (e.g., cardiac, gastrointestinal [GI], respiratory, skin, central nervous) and multiple symptoms (e.g., high fevers, fatigue, anorexia, nausea, vomiting, rash, hypotension, hypoxia, headache, delirium, confusion). CRS may be life-threatening. Clinically, CRS can be mistaken for a systemic infection or, in severe cases, septic shock. Frequently the earliest sign is elevated temperature, which should prompt an immediate differential diagnostic work-up and timely initiation of appropriate treatment.

The goal of CRS management is to prevent life-threatening states and sequelae while preserving the potential for the anticancer effects of CTX130. Symptoms usually occur 1 to 14 days after autologous CAR T cell therapy in hematologic malignancies.

CRS should be identified and treated based on clinical presentation and not laboratory measurements. If CRS is suspected, grading should be applied according to the ASTCT (formerly known as American Society for Blood and Marrow Transplantation, ASBMT) consensus recommendations (Table 29; Lee et al., (2019) Biol Blood Marrow Transplant 25, 625-638), and management should be performed according to the recommendations in Table 30, which are adapted from published guidelines (Lee et al., (2014) Blood 124, 188-95; Lee et al., (2019) Biol Blood Marrow Transplant 25, 625-638). Accordingly, grading of neurotoxicity is aligned with the ASTCT criteria for ICANS.

TABLE 29
Grading of CRS According to the ASTCT Consensus Criteria

| CRS | | | | |
|---|---|---|---|---|
| Parameter | Grade 1 | Grade 2 | Grade 3 | Grade 4 |
| Fever ^{a} | Temper- | Temper- | Temper- | Temper- |
| | ature ≥ | ature ≥ | ature ≥ | ature ≥ |
| | 38° C. | 38° C. | 38° C. | 38° C. |
| With | None | Not requiring | Requiring a vasopressor | Requiring multiple |
| Hypotension | | vasopressors | with or without | vasopressors |
| | | | vasopressin ^{b} | (excluding |
| | | | | vasopressin) ^{b} |
| And/or ^{c} | None | Requiring low-flow | Requiring high-flow | Requiring positive |
| Hypoxia | | nasal cannula ^{d} or | nasal cannula ^{d}, | pressure (e.g., CPAP, |
| | | blow-by | facemask, | BiPAP, intubation, and |
| | | | nonrebreather mask, or | mechanical ventilation |
| | | | Venturi mask | |

ASTCT: American Society for Transplantation and Cellular Therapy;
BiPAP: bilevel positive airway pressure;
C = Celsius;
CPAP: continuous positive airway pressure;
CRS: cytokine release syndrome.
^{a} Fever is defined as temperature ≥ 38° C. not attributable to any other cause. In patients who have CRS then receive antipyretics or anticytokine therapy such astocilizumab or steroids, fever is no longer required to grade subsequent CRS severity. In this case, CRS grading is driven by hypotension and/or hypoxia.
^{b} See Table 31 for information on high-dose vasopressors.
^{c} CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as Grade 3 CRS.
^{d} Low-flow nasal cannula is defined as oxygen delivered at ≤ 6 L/minute. Low flow also includes blow-by oxygen delivery, sometimes used in pediatrics. High-flow nasal cannula is defined as oxygen delivered at > 6 L/minute.
Note:
Organ toxicities associated with CRS may be graded according to CTCAE v5.0 but they do not influence CRS grading.

TABLE 30
Cytokine Release Syndrome Grading and Management Guidance.

| CRS | | | Hypotension |
|---|---|---|---|
| Severity ¹ | Tocilizumab | Corticosteroids | Management |
| Grade 1 | Tocilizumab ² may be | N/A | N/A |
| | considered | | |
| Grade 2 | Administer tocilizumab 8 | Manage per institutional guidelines | Manage per |
| | mg/kg IV over 1 hour (not to | if no improvement after initial | institutional |
| | exceed 800 mg). ² | tocilizumab therapy. Continue | guidelines. |
| | Repeat tocilizumab every 8 | corticosteroids use until the event is | |
| | hours as needed if not | Grade ≤ 1, then taper appropriately. | |
| | responsive to IV fluids or | | |
| | increasing supplemental | | |
| | oxygen. | | |
| | Limit to ≤ 3 doses in a 24-hour | | |
| | period; maximum total of 4 | | |
| | doses. | | |
| Grade 3 | Per grade 2. | Per grade 2. | Manage per |
| | | | institutional |
| | | | guidelines. |
| Grade 4 | Per grade 2. | Per grade 2. | Manage per |
| | If no response to multiple | | institutional |
| | doses of tocilizumab and | | guidelines. |
| | steroids, consider using other | | |
| | anti-cytokine therapies (e.g., | | |
| | anakinra). | | |

CRS: cytokine release syndrome;
IV: intravenously;
N/A: not applicable.
¹ See (Lee et al., (2019) Biol Blood Marrow Transplant 25, 625-638)
² Refer to tocilizumab prescribing information.

TABLE 31
High-dose Vasopressors

| Pressor | Dose* |
|---|---|
| Norepinephrine monotherapy | ≥20 μg/min |
| Dopamine monotherapy | ≥10 μg/kg/min |
| Phenylephrine monotherapy | ≥200 μg/min |
| Epinephrine monotherapy | ≥10 μg/min |
| If on vasopressin | Vasopressin + norepinephrine |
| | equivalent of ≥ 10 μg/min** |
| If on combination vasopressors | Norepinephrine equivalent |
| (not vasopressin) | of ≥ 20 μg/min** |

*All doses are required for ≥ 3 hours.
**VASST Trial vasopressor equivalent equation: norepinephrine equivalent dose = [norepinephrine (μg/min)] + [dopamine (μg/min)/2] + [epinephrine (μg/min)] + [phenylephrine (μg/min)/10].

Throughout the duration of CRS, subjects should be provided with supportive care consisting of antipyretics, IV fluids, and oxygen. Subjects who experience Grade≥2 CRS should be monitored with continuous cardiac telemetry and pulse oximetry. For subjects experiencing Grade 3 CRS, consider performing an echocardiogram to assess cardiac function. For Grade 3 or 4 CRS, consider intensive care supportive therapy. The potential of an underlying infection in cases of severe CRS should be considered, as the presentation (fever, hypotension, hypoxia) is similar. Resolution of CRS is defined as resolution of fever (temperature≥38° C.), hypoxia, and hypotension (Lee et al., (2019) Biol Blood Marrow Transplant 25, 625-638).

### 8.2.4.1 Hypotension and Renal Insufficiency

Hypotension and renal insufficiency have been reported with CAR T cell therapy and should be treated with IV administration of normal saline boluses according to institutional practice guidelines. Dialysis should be considered when appropriate.

### 8.2.5 Immune Effector Cell-Associated Neurotoxicity Syndrome (ICANS)

Neurotoxicity has been documented in subjects with B cell malignancies treated with autologous CAR T cell therapies. Neurotoxicity may occur at the time of CRS, during the resolution of CRS, or following resolution of CRS, and its pathophysiology is unclear. The recent ASTCT (formerly known as ASBMT) consensus further defined ICANS as a disorder characterized by a pathologic process involving the CNS following any immune therapy that results in activation or engagement of endogenous or infused T cells and/or other immune effector cells (Lee et al., (2019) Biol Blood Marrow Transplant 25, 625-638). Signs and symptoms can be progressive and may include but are not limited to aphasia, altered level of consciousness, impairment of cognitive skills, motor weakness, seizures, and cerebral edema. ICANS grading (Table 32) was developed based on CAR T cell therapy-associated TOXicity (CARTOX) working group criteria used previously in autologous CAR T cell trials (Neelapu et al., (2018) Nat Rev Clin Oncol 15, 47-62). ICANS incorporates assessment of level of consciousness, presence/absence of seizures, motor findings, presence/absence of cerebral edema, and overall assessment of neurologic domains by using a modified tool called the immune effector cell-associated encephalopathy (ICE) assessment tool (Table 17).

Evaluation of any new onset neurotoxicity should include a neurological examination (including ICE assessment tool, Table 17), brain magnetic resonance imaging (MRI), and examination of the CSF, as clinically indicated. If clinically feasible, for lumbar punctures performed during neurotoxicity, CSF samples should be sent to the central laboratory for exploratory biomarkers and for presence of CTX130 (by PCR). If a brain MRI is not possible, all subjects should receive a noncontrast computed tomography (CT) scan to rule out intracerebral hemorrhage. Electroencephalogram should also be considered as clinically indicated. Endotracheal intubation may be needed for airway protection in severe cases.

Nonsedating, antiseizure prophylaxis (e.g., levetiracetam) should be considered, especially in subjects with a history of seizures, for at least 28 days following CTX130 infusion or upon resolution of neurological symptoms (unless the antiseizure medication is considered to contribute to the detrimental symptoms). Subjects who experience Grade≥2 ICANS should be monitored with continuous cardiac telemetry and pulse oximetry. For severe or life-threatening neurologic toxicities, intensive care supportive therapy should be provided. Neurology consultation should always be considered. Monitor platelets and for signs of coagulopathy, and transfuse blood products appropriately to diminish risk of intracerebral hemorrhage. Table 32 provides neurotoxicity grading and Table 33 provides management guidance.

For subjects who receive active steroid management for more than 3 days, antifungal and antiviral prophylaxis is recommended to mitigate a risk of severe infection with prolonged steroid use. Consideration for antimicrobial prophylaxis should also be given.

TABLE 32
ICANS Grading.

| Neurotoxicity | | | | |
|---|---|---|---|---|
| Domain | Grade 1 | Grade 2 | Grade 3 | Grade 4 |
| ICE score ¹ | 7-9 | 3-6 | 0-2 | 0 (subject is unarousable |
| | | | | and unable to undergo |
| | | | | ICE assessment) |
| Depressed | Awakens | Awakens | Awakens only to | Subject is unarousable or |
| level of | spontaneously | to voice | tactile stimulus | requires vigorous or |
| consciousness ² | | | | repetitive tactile stimuli |
| | | | | to arise; stupor or coma |
| Seizure | N/A | N/A | Any clinical seizure, | Life-threatening |
| | | | focal or generalized, | prolonged seizure (>5 |
| | | | that resolves rapidly, | min) or repetitive |
| | | | or nonconvulsive | clinical or electrical |
| | | | seizures on EEG that | seizures without return |
| | | | resolve with | to baseline in between |
| | | | intervention | |
| Motor findings ³ | N/A | N/A | N/A | Deep focal motor |
| | | | | weakness such as |
| | | | | hemiparesis or |
| | | | | paraparesis |
| Elevated ICP/ | N/A | N/A | Focal/local edema on | Diffuse cerebral edema |
| cerebral edema | | | neuroimaging ⁴ | on neuroimaging, |
| | | | | decerebrate or |
| | | | | decorticate posturing, |
| | | | | cranial nerve VI palsy, |
| | | | | papilladema, or |
| | | | | Cushing’s triad |

CTCAE: Common Terminology Criteria for Adverse Events;
EEG: electroencephalogram;
ICANS: immune effector cell-associated neurotoxicity syndrome;
ICE: immune effector cell-associated encephalopathy (assessment tool);
ICP: intracranial pressure;
N/A: not applicable.
ICANS grade is determined by the most severe event (ICE score, level of consciousness, seizure, motor findings, raised ICP/cerebral edema) not attributable to any other cause.
¹ A subject with an ICE score of 0 may be classified as grade 3 ICANS if awake with global aphasia, but a subject with an ICE score of 0 may be classified as grade 4 ICANS if unarousable.
² Depressed level of consciousness should be attributable to no other cause (e.g., sedating medication).
³ Tremors and myoclonus associated with immune effector therapies should be graded according to CTCAE v5.0 but do not influence ICANS grading.

TABLE 33
ICANS Management Guidance.

| Severity | Management |
|---|---|
| Grade 2 | Consider administering dexamethasone 10 mg IV every 6 hours (or equivalent |
| | methylprednisolone) unless subject already on equivalent dose of steroids for |
| | CRS. |
| | Continue dexamethasone use until event is grade ≤ 1, then taper over 3 days. |
| Grade 3 | Administer dexamethasone 10 mg IV every 6 hours, unless subject already on |
| | equivalent dose of steroids for CRS. |
| | Continue dexamethasone use until event is grade ≤ 1, then taper over 3 days. |
| Grade 4 | Administer methylprednisolone 1000 mg IV per day for 3 days; if improves, |
| | manage as above. |

CRS: cytokine release syndrome;
ICANS: immune effector cell-associated neurotoxicity syndrome;
IV: intravenously.

Headache, which may occur in a setting of fever or after chemotherapy, is a nonspecific symptom. Headache alone may not necessarily be a manifestation of ICANS and further evaluation should be performed. Weakness or balance problem resulting from deconditioning and muscle loss are excluded from definition of ICANS. Similarly, intracranial hemorrhage with or without associated edema may occur due to coagulopathies in these subjects and are also excluded from definition of ICANS. These and other neurotoxicities should be captured in accordance with CTCAE v5.0.

### 8.2.6 Hemophagocytic Lymphohistiocytosis (HLH)

HLH has been reported after treatment with autologous CD19-directed CAR T cells (Barrett et al., (2014) Curr Opin Pediatr, 26, 43-49; Maude et al., (2014) N Engl J Med, 371, 1507-1517; Maude et al., (2015) Blood, 125, 4017-4023; Porter et al., (2015) Sci Transl Med, 7, 303ra139; Teachey et al., (2013) Blood, 121, 5154-5157. HLH is a clinical syndrome that is a result of an inflammatory response following infusion of CAR T cells in which cytokine production from activated T cells leads to excessive macrophage activation. Signs and symptoms of HLH may include fevers, cytopenias, hepatosplenomegaly, hepatic dysfunction with hyperbilirubinemia, coagulopathy with significantly decreased fibrinogen, and marked elevations in ferritin and C-reactive protein (CRP). Neurologic findings have also been observed (Jordan et al., (2011) Blood, 118, 4041-4052; La Rosée, (2015) Hematology Am Soc Hematol Educ Program, 190-196.

CRS and HLH may possess similar clinical syndromes with overlapping clinical features and pathophysiology. HLH likely occurs at the time of CRS or as CRS is resolving. HLH should be considered if there are unexplained elevated liver function tests or cytopenias with or without other evidence of CRS. Monitoring of CRP and ferritin may assist with diagnosis and define the clinical course.

If HLH is suspected:
Frequently monitor coagulation parameters, including fibrinogen. These tests may be done more frequently than indicated in the schedule of assessments, and frequency should be driven based on laboratory findings.
Fibrinogen should be maintained ≥100 mg/dL to decrease risk of bleeding.
Coagulopathy should be corrected with blood products.
Given the overlap with CRS, subjects should also be managed per CRS treatment guidance in Table 29.

### 8.2.7 Prolonged Cytopenias

Grade 3 neutropenia and thrombocytopenia, at times lasting more than 28 days after CAR T cell infusion, have been reported in subjects treated with autologous CAR T cell products (Kymriah US prescribing information [USPI], 2018; Raje et al., (2019) N Engl J Med 380, 1726-37; Yescarta USPI, 2019). Therefore, subjects receiving CTX130 should be monitored for such toxicities and appropriately supported. Monitor platelets and for signs of coagulopathy and transfuse blood products appropriately to diminish risk of hemorrhage. Consideration should be given to antimicrobial and antifungal prophylaxis for any subject with prolonged neutropenia.

Due to the transient expression of CD70 on activated T and B lymphocytes, opportunistic infection such as viral reactivation may occur. Opportunistic infections shall be considered when clinical symptoms arise.

During dose escalation, G-CSF may be considered in cases of Grade 4 neutropenia post-CTX130 infusion. During cohort expansion G-CSF may be administered cautiously per healthcare practitioner's discretion.

### 8.2.8 Graft Versus Host Disease (GvHD)

GvHD is seen in the setting of allogeneic HSCT and is the result of immunocompetent donor T cells (the graft) recognizing the recipient (the host) as foreign. The subsequent immune response activates donor T cells to attack the recipient to eliminate foreign antigen-bearing cells. GvHD is divided into acute, chronic, and overlap syndromes based on both the time from allogeneic HSCT and clinical manifestations. Signs of acute GvHD may include a maculopapular rash; hyperbilirubinemia with jaundice due to damage to the small bile ducts, leading to cholestasis; nausea, vomiting, and anorexia; and watery or bloody diarrhea and cramping abdominal pain (Zeiser and Blazar, (2017) N Engl J Med, 377, 2167-2179).

To support the proposed clinical study, a nonclinical GLP-compliant GvHD and tolerability study was performed in immunocompromised mice treated at 2 IV doses: a high dose of 4×10⁷ CTX130 cells per mouse (approximately 1.6×10⁹ cells/kg) and a low dose of 2×10⁷ cells per mouse (approximately 0.8×10⁹ cells/kg). Both dose levels exceed the proposed highest clinical dose by more than 10-fold when normalized for body weight. No mice treated with CTX130 developed fatal GvHD during the course of the 12-week study. At necropsy, mononuclear cell infiltration was observed in some animals in the mesenteric lymph node and the thymus. Minimal to mild perivascular inflammation was also observed in the lungs of some animals. These findings are consistent with mild GvHD, but did not manifest in clinical symptoms in these mice.

Further, due to the specificity of CAR insertion at the TRAC locus, it is highly unlikely for a T cell to be both CAR+ and TCR+. Remaining TCR+ cells are removed during the manufacturing process by immunoaffinity chromatography on an anti-TCR antibody column to achieve ≤0.4% TCR+ cells in the final product. A dose limit of 7×10⁴ TCR+ cells/kg is imposed for all dose levels. This limit is lower than the limit of 1×10⁵ TCR+ cells/kg based on published reports on the number of allogeneic cells capable of causing severe GvHD during SCT with haploidentical donors (Bertaina et al., (2014) Blood, 124, 822-826). Through this specific editing, purification, and strict product release criteria, the risk of GvHD following CTX130 should be low, although the true incidence is unknown. However, given that CAR T cell expansion is antigen-driven and is likely occur only in TCR− cells, it is unlikely that the number of TCR+ cells can be appreciably increase above the number infused.

Diagnosis and grading of GvHD should be based on published criteria (Harris et al., (2016) Biol Blood Marrow Transplant, 22, 4-10), as outlined in Table 34.

TABLE 34
Criteria for Grading Acute GvHD

| | | Liver | | |
|---|---|---|---|---|
| | Skin | (bilirubin | | Lower GI (stool |
| Stage | (active erythema only) | mg/dL) | Upper GI | output/day) |
| 0 | No active (erythematous) | <2 | No or intermittent | <500 ml/day or < 3 |
| | GvHD rash | | nausea, vomiting, | episodes/day |
| | | | or anorexia | |
| 1 | Maculopapular rash < | 2-3 | Persistent nausea, | 500-999 ml/day or |
| | 25% BSA | | vomiting, or | 3-4 episodes/day |
| | | | anorexia | |
| 2 | Maculopapular rash | 3.1-6 | — | 1000-1500 ml/day or |
| | 25-50% BSA | | | 5-7 episodes/day |
| 3 | Maculopapular | 6.1-15 | — | >1500 ml/day or > |
| | rash > 50% BSA | | | 7 episodes/day |
| 4 | Generalized erythroderma | >15 | — | Severe abdominal pain |
| | (>50% BSA) plus bullous | | | with or without ileus, or |
| | formation and | | | grossly bloody stool |
| | desquamation > 5% BSA | | | (regardless of stool |
| | | | | volume) |

BSA: body surface area;
GI: gastrointestinal;
GvHD: graft versus host disease.

Overall GvHD grade can be determined based on most severe target organ involvement.
Grade 0: No stage 1-4 of any organ.
Grade 1: Stage 1-2 skin without liver, upper GI, or lower GI involvement.
Grade 2: Stage 3 rash and/or stage 1 liver and/or stage 1 upper GI and/or stage 1 lower GI.
Grade 3: Stage 2-3 liver and/or stage 2-3 lower GI, with stage 0-3 skin and/or stage 0-1 upper GI.
Grade 4: Stage 4 skin, liver, or lower GI involvement, with stage 0-1 upper GI.

Potential confounding factors that may mimic GvHD such as infections and reactions to medications should be ruled out. Skin and/or GI biopsy should be obtained for confirmation before or soon after treatment has been initiated. In instance of liver involvement, liver biopsy should be attempted if clinically feasible.

Recommendations for management of acute GvHD are outlined in Table 35. To allow for intersubject comparability at the end of the trial, these recommendations should be followed except in specific clinical scenarios in which following them could put the subject at risk.

TABLE 35
Acute GvHD Management

| Grade | Management |
|---|---|
| 1 | Skin: Topical steroids or immunosuppressants; if stage 2: prednisone 1 mg/kg |
| | (or equivalent dose). |
| 2-4 | Initiate prednisone 2 mg/kg daily (or equivalent dose). |
| | IV form of steroid such as methylprednisolone should be considered if there |
| | are concerns with malabsorption. |
| | Steroid taper may begin after improvement is seen after ≥ 3 days of steroids. |
| | Taper should be 50% decrease of total daily steroid dose every 5 days. |
| | GI: In addition to steroids, start anti-diarrheal agents per standard practice. |

GI: gastrointestinal;
IV: intravenous.

Decisions to initiate second-line therapy should be made sooner for subjects with more severe GvHD. For example, secondary therapy may be indicated after 3 days with progressive manifestations of GvHD, after 1 week with persistent grade 3 GvHD, or after 2 weeks with persistent grade 2 GvHD. Second-line systemic therapy may be indicated earlier in subjects who cannot tolerate high-dose glucocorticoid treatment (Martin et al., (2012) Biol Blood Marrow Transplant, 18, 1150-1163). Choice of secondary therapy and when to initiate can be based on clinical judgement and local practice.

Management of refractory acute GvHD or chronic GvHD can be per institutional guidelines. Anti-infective prophylaxis measures should be instituted per local guidelines when treating subjects with immunosuppressive agents (including steroids).

### 8.2.9 On-Target Off-Tumor Toxicities

### 8.2.9.1 Activity of CTX130 Against Activated T and B Lymphocytes, Dendritic Cells

Activated T and B lymphocytes express CD70 transiently and dendritic cells, as well as thymic epithelial cells, express CD70 to a certain degree. Thus, these cells might become a target for activated CTX130.

### 8.2.9.2 Activity of CTX130 Against Osteoblasts

Activity of CTX130 was detected in nonclinical studies in cell culture of human primary osteoblasts. Hence, bone turnover is monitored via calcium levels as well as 2 osteoblast-specific markers, amino-terminal propeptide of type I procollagen (PINP) and bone-specific alkaline phosphatase (BSAP), which are considered the most useful markers in the assessment of bone formation (Fink et al., (2000) Osteoporosis 11, 295-303). Standardized assays for assessment of both markers in serum are available. The concentration of these peptide markers reflects the activity of osteoblasts and the formation of new bone collagen. PINP and BSAP are measured through a central laboratory assessment at screening, baseline, Days 7, 14, 21, and 28, and Months 3, 6, and 12 of the study (Table 15). Samples are to be collected at the same time of day (±2 hours) on the specified collection days because of the strong effect of circadian rhythm on bone turn over.

### 8.2.9.3 Activity of CTX130 Against Renal Tubular-Like Epithelium

Activity of CTX130 against renal tubular-like epithelial cells was detected in nonclinical studies of CTX130 in primary human kidney epithelium. Hence, subjects should be monitored for acute tubular damage by monitoring for an increase in serum creatinine of at least 0.3 mg/dL (26.5 μmol/L) over a 48-hour period and/or ≥1.5 times the baseline value within the previous 7 days. Serum creatinine is assessed daily for the first 7 days post-CTX130 infusion, every other day between Days 8 through 14 of treatment, and then twice weekly until Day 28 (Table 14). If acute renal tubular damage is suspected, additional tests should be conducted including urine sediment analysis and fractional excretion of sodium in urine, and consultation by a nephrologist should be initiated.

### 9. Statistical Methods

### 9.1 Sample Size

In Part A (dose escalation, the sample size is approximately 6 to 24 DLT-evaluable subjects, depending on the number of dose levels evaluated and the occurrence of DLTs.

In Part B (cohort expansion), a Simon's 2-stage Minimax design can used and up to 21 subjects with DLBCL can be enrolled.

### 9.2 Analysis Sets

### Part A (Dose Escalation)

The DLT-evaluable set includes all subjects who receive CTX130 and are followed for at least 28 days post infusion or after experiencing a DLT.

### Part A+Part B

Safety analysis set (SAS): All subjects who were enrolled and received at least 1 dose of study treatment. Subjects are classified according to the treatment received, where treatment received is defined as the assigned dose level/schedule if it was received at least once, or the first dose level/schedule received if assigned treatment was never received. The SAS is the primary set for the analysis of safety data.
Full analysis set (FAS): All subjects who were enrolled and received CTX130 infusion and have at least 1 baseline and 1 post-baseline scan assessment. The FAS is the primary analysis set for clinical activity assessment.

### 9.3 Endpoints

### 9.3.1 Primary Endpoints

Part A (Dose Escalation): The incidence of adverse events (AEs), defined as dose-limiting toxicities (DLTs), and definition of RPBD.
Part B (Cohort Expansion): The objective response rate (ORR) as per (complete response [CR]+partial response [PR]) according to the Lugano response criteria (Cheson et al., (2014) J Clin Oncol 32, 3059-68) for subjects with DLBCL as assessed by an independent central radiology review.

### 9.3.2 Secondary Endpoints

### 8.3.2.1 Efficacy

Part A: Efficacy assessments per Lugano response criteria (Cheson et al., (2014) J Clin Oncol 32, 3059-68) for subjects with PTCL-NOS, ALCL, leukemic and lymphomatous ATLL, AITL, and DLBCL, and per ISCL response criteria (Olsen et al., 2011) for subjects with SS or MF;

Part B: Efficacy assessments per Lugan response criteria (Cheson et al., (2014) J Clin Oncol 32, 3059-68) for subjects with DLBCL:
Subject best response (complete response (CR), partial response (PR), stable disease (SD), progressive disease (PD), or not evaluable (NE)).
Objective response rate (ORR), defined as the percentage of subjects who achieved CR or PR.
Time to response (TTR), defined as the time between the date of CTX130 infusion until first documented response (PR/CR).
Duration of response (DoR), defined as the time between first objective response of PR/CR and date of disease progression or death due to any cause. Reported only for subjects who have had PR/CR events.
Progression-free survival (PFS), defined as the difference between date of CTX130 infusion and date of disease progression or death due to any cause.
Overall survival (OS), defined as the time between date of CTX130 infusion and death due to any cause.
Disease control rate (DCR), defined as the percentage of subjects who achieved CR, PR, or SD.
Time to progression (TTP), defined as the difference between date of CTX130 infusion and date of PD.

### 9.3.2.2 Safety

Incidence and severity of AEs and clinically significant laboratory abnormalities.

### 9.3.2.3 Pharmacokinetics

Levels of CTX130 in blood over time.

### 9.3.2.4 Exploratory Endpoints (Parts A and B)

Levels of CTX130 in tissues.
Levels of cytokines in blood and other tissues.
Incidence of anti-CTX130 antibodies.
Impact of anti-cytokine therapy on CTX130 proliferation, CRS, and response.
Incidence of autologous or allogeneic hematopoietic stem cell transplantation (HSCT) following CTX130 therapy.
Incidence and type of subsequent anti-cancer therapy.
Time to complete response (CR), defined as the time between the date of CTX130 infusion until first documented CR.
Time to disease progression (PD), defined as time between the date of CTX130 infusion until first evidence of disease progression.
Changes in peripheral blood levels of ATLL cells as monitored by immunophenotyping based on markers such as CD3, CD4, CD7, CD8, CD25, CD52, and HTLV-1 proviral load.
Response assessment and concordance rate with central review.
First subsequent therapy free survival, defined as the time between date of CTX130 infusion and date of first subsequent therapy or death due to any cause.
Change from baseline in in PRO, as measured by European Organization for Research and Treatment of Cancer (EORTC) QLQ-30, EQ-5D-5L questionnaires, FACT-G, Skindex-29 questionnaire for SS and MF, and Dermatology Life Quality Index (DLQI) questionnaire for SS and MF.
Change from baseline in cognitive outcome, as assessed by ICE.
Other genomic, protein, metabolic, or pharmacodynamic endpoints.

### Results

To date, all subjects that participated in this study have completed Stage 1 (eligibility screening) within 14 days. After having met the eligibility criteria, two subjects started lymphodepleting therapy within 24 hours of completing Stage 1. All eligible subjects have completed the screening period (stage 1) and started LD chemotherapy in less than 8 days, with one subject completing screening and starting an LD chemo dose within 72 hrs. One subject receiving LD chemotherapy has already progressed to receiving the DL1 dose of CTX130 within 5 days following completion of the LD chemotherapy.

None of the treated subjects in this study exhibited any DLTs so far. Similarly, no DTLs were observed in a parallel study using CTX130 to treat subjects with RCC. See, e.g., U.S. Patent Application No. 62/934,961 filed Nov. 13, 2019 and U.S. Patent Application No. 63/034,552 filed Jun. 4, 2020. Further, the allogeneic CAR T cell therapy exhibited desired pharmacokinetic features in the treated human subjects, including CAR T cell expansion and persistence after infusion. Significant CAR T cell distribution, expansion and persistence has been observed as early as DL1. Up to 20-fold expansion of CTX130 in peripheral blood over To has been observed in one T-cell lymphoma subject evaluated to date and persistence of CTX130 cells were detected in DL1 subjects up to 14 days post-infusion. Similar patterns of CAR T cell distribution, expansion and persistence are observed in the corresponding CTX130 RCC study, where 87-fold expansion of CTX130 has been observed and CTX130 cells have been detected for at least 28 days following infusion.

The eligible subjects in this study has MF with large cell transformation. Results obtained from the first T-cell lymphoma subject are summarized below.
The subject receiving the DL1 dose experienced significant reduction of the skin lesions as documented per photography according to the Olson/ISCL criteria for cutaneous T-cell lymphoma response assessment. Furthermore, a PET/CT scan 4 weeks following CTX130 infusion in the same subject revealed a drastic decrease in nodal and cutaneous lesions with most lesions entirely disappeared qualifying for a formal partial metabolic response.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

1. A method for treating a hematopoietic cell malignancy, the method comprising:
(i) subjecting a human patient having a hematopoietic cell malignancy to a first lymphodepletion treatment;
(ii) administering to the human patient a first dose of a population of genetically engineered T cells after step (i), wherein the population of genetically engineered T cells comprises T cells expressing a chimeric antigen receptor (CAR) that binds CD70, a disrupted TRAC gene, a disrupted β2M gene, and a disrupted CD70 gene, and wherein a nucleotide sequence encoding the CAR is inserted into the disrupted TRAC gene.

2. The method of claim 1, wherein the first lymphodepletion treatment in step (i) comprises co-administering to the human patient fludarabine at 30 mg/m² and cyclophosphamide at 500 mg/m² intravenously per day for three days.

3. The method of claim 1, wherein prior to step (i), the human patient does not show one or more of the following features:
(a) change in performance status to ECOG>1,
(b) significant worsening of clinical status,
(c) requirement for supplemental oxygen to maintain a saturation level of greater than 92%,
(d) uncontrolled cardiac arrhythmia,
(e) hypotension requiring vasopressor support,
(f) active infection, and
(g) any acute neurological toxicity.

4. The method of claim 1, wherein step (i) is performed about 2-7 days prior to step (ii).

5. The method of claim 1, wherein step (ii) is performed by administering the population of genetically engineered T cells to the human patient intravenously at the first dose, which is about 1×10⁷ CAR⁺ cells to about 1×10⁹ CAR⁺ cells, optionally about 3×10⁷ to about 9×10⁸ CAR+ cells.

6. The method of claim 1, wherein prior to step (ii) and after step (i), the human patient does not show one or more of the following features:
(a) change in performance status to Eastern Cooperative Oncology Group (ECOG)>1,
(b) active uncontrolled infection,
(c) significant worsening of clinical status, and
(d) any acute neurological toxicity.

7. The method of claim 1, further comprising (iii) monitoring the human patient for development of acute toxicity after step (ii).

8. The method of claim 7, wherein acute toxicity comprises cytokine release syndrome (CRS), neurotoxicity, tumor lysis syndrome, GvHD, on target off-tumor toxicity, and/or uncontrolled T cell proliferation.

9. The method of claim 1, further comprising (iv) subjecting the human patient to a second lymphodepletion treatment, and (v) administering to the human patient a second dose of the population of genetically engineered T cells after step (ii), optionally wherein the second dose is administered about 8 weeks to about 2 years after the first dose, and optionally wherein the human patient does not show one or more of the following after step (ii):
(a) dose-limiting toxicity (DLT),
(b) grade 4 CRS that does not resolve to grade 2 within 72 hours,
(c) grade>1 GvHD,
(d) grade≥3 neurotoxicity
(e) active infection,
(f) hemodynamically unstable, and
(g) organ dysfunction.

10. The method of claim 9, wherein the second lymphodepletion treatment in step (iv) comprises co-administering to the human patient fludarabine at 30 mg/m² and cyclophosphamide at 500 mg/m² intravenously per day for 1-3 days.

11. The method of claim 9, wherein step (v) is performed 2-7 days after step (iv).

12. The method of claim 9, wherein step (v) is performed by administering the population of genetically engineered T cells to the human patient intravenously at the second dose, which is about 1×10⁷ CAR⁺ cells to about CAR⁺ 1×10⁹ cells, optionally about 3×10⁷ to about 9×10⁸ CAR+ cells.

13. The method of claim 9, wherein the method further comprises (vi) subjecting the human patient to a third lymphodepletion treatment, and (vii) administering to the human patient a third dose of the population of genetically engineered T cells, optionally wherein the human patient receives the first, second, and third doses of the population of genetically engineered T cells in three months, and optionally wherein the human patient does not show one or more of the following after step (v):
(a) dose-limiting toxicity (DLT),
(b) grade 4 CRS that does not resolve to grade 2 within 72 hours,
(c) grade≥1 GvHD,
(d) grade≥3 neurotoxicity
(e) active infection,
(f) hemodynamically unstable, and
(g) organ dysfunction.

14. The method of claim 13, wherein the third lymphodepletion treatment in step (vi) comprises co-administering to the human patient fludarabine at 30 mg/m² and cyclophosphamide at 500 mg/m² intravenously per day for 1-3 days.

15. The method of claim 13, wherein step (vii) is performed 2-7 days after step (vi).

16. The method of claim 13, wherein step (vii) is performed by administering the population of genetically engineered T cells to the human patient intravenously at the third dose, which is about 1×10⁷ CAR⁺ cells to about 1×10⁹ CAR⁺ cells, optionally about 3×10⁷ to about 9×10⁸ CAR+ cells.

17. The method of claim 9, wherein the human patient shows stable disease or disease progress.

18. The method of claim 1, wherein the first dose, the second dose, and/or the third dose of the population of genetically engineered T cells is 1×10⁷ CAR⁺ cells, 3×10⁷ CAR⁺ cells, 1×10⁸ CAR⁺ cells, 3×10⁸ CAR⁺ cells, or 1×10⁹ CAR⁺ cells, optionally wherein the first dose, the second dose, and/or the third dose of the population of genetically engineered T cells is 1.5×10⁸ CAR⁺ cells, 4.5×10⁸ CAR⁺ cells, 6×10⁸ CAR⁺ cells, 7.5×10⁸ CAR⁺ cells, or 9×10⁸ CAR⁺ cells.

19. The method of claim 9, wherein the first dose of the population of genetically engineered T cells is the same as the second and/or third dose of the population of genetically engineered T cells.

20. The method of claim 9, wherein the first dose of the population of genetically engineered T cells is lower than the second and/or third dose of the population of genetically engineered T cells.

21. The method of claim 1, wherein the human patient has undergone a prior anti-cancer therapy.

22. The method of claim 1, wherein the human patient has relapsed or refractory hematopoietic cell malignancies.

23. The method of claim 1, wherein the human patient has a T cell malignancy, which optionally is selected from the group consisting of cutaneous T-cell lymphoma (CTCL), peripheral T-cell lymphoma (PTCL), and T cell leukemia.

24. The method of claim 23, wherein the CTCL is Sezary Syndrome (SS) or mycosis fungoides (MF), wherein optionally the human patient has Stage IIb or higher MF, optionally transformed large cell lymphoma.

25. The method of claim 23, wherein the PTCL is angioimmunoblastic T cell lymphoma (AITL), anaplastic large cell lymphoma (ALCL), adult T cell leukemia or lymphoma (ATLL), or PTCL not otherwise (PTCL-NOS).

26. The method of claim 25, wherein the human patient has PTCL, ATLL, or AITL and has failed a first line systemic therapy.

27. The method of claim 25, wherein the human patient has ALCL and has failed a combined therapy comprising breutuximab vedotin.

28. The method of claim 27, wherein the human patient has ALK⁺ ALCL and has failed two prior lines of therapy, one of which comprises brentuximab vedotin.

29. The method of claim 27, wherein the human patient has ALK⁻ ALCL and has failed one prior line of therapy.

30. The method of claim 24, wherein the human patient has MF or SS and has failed a prior systemic therapy or a prior mogamulizumab therapy.

31. The method of claim 1, wherein the human patient has a B cell malignancy, which optionally is diffuse large B cell lymphoma (DLBCL) or mantle cell lymphoma (MCL).

32. The method of claim 31, wherein the human patient has DLBCL and has failed a prior anti-CD19 CAR-T cell therapy.

33. The method of claim 1, wherein the human patient has a myeloid cell malignancy, which optionally is acute myeloid leukemia (AML).

34. The method of claim 1, wherein the human patient is free of mogamulizumab treatment at least three months prior to the first dose of the population of genetically modified T cells.

35. The method of claim 1, wherein the human patient has CD70+ tumor cells.

36. The method of claim 35, wherein the human patient has at least 10% CD70⁺ tumor cells in a biological sample obtained from the human patient.

37. The method of claim 36, wherein the biological sample is a tumor tissue sample and the level of CD70+ tumor cells is measured by immunohistochemistry (IHC).

38. The method of claim 37, wherein the biological sample is a blood sample or a bone marrow sample and the level of CD70+ tumor cells is determined by flow cytometry.

39. The method of claim 35, wherein the method further comprising, prior to step (i), identifying a human patient having CD70+ tumor cells involved in a hematopoietic cell malignancy, which optionally is a T cell malignancy, a B cell malignancy, or a myeloid cell malignancy.

40. The method of claim 1, wherein the human patient is subject to an anti-cytokine therapy.

41. The method of claim 1, wherein the human patient has one or more of the following features:
(a) adequate organ function,
(b) free of a prior stem cell transplantation (SCT),
(c) free of a prior anti-CD70 agent or adoptive T cell or NK cell therapy,
(d) free of known contraindication to a lymphodepletion therapy,
(e) free of T cell or B cell lymphomas with a present or a past malignant effusion that is or was symptomatic,
(f) free of hemophagocytic lymphohistiocytosis (HLH),
(g) free of central nervous system malignancy or disorders,
(h) free of unstable angina, arrhythmia, and/or myocardial infarction,
(i) free of diabetes mellitus,
(j) free of uncontrolled infections,
(k) free of immunodeficiency disorders or autoimmune disorders that require immunosuppressive therapy, and
(l) free of solid organ transplantation.

42. The method of claim 1, wherein the human patient is monitored for at least 28 days for development of toxicity after each administration of the population of genetically engineered T cells.

43. The method of claim 42, wherein the human patient is subject to toxicity management when development of toxicity is observed.

44. The method of claim 1, wherein the human patient is an adult.

45. The method of claim 1, wherein the CAR that binds CD70 comprises an extracellular domain, a CD8 transmembrane domain, a 4-1BB co-stimulatory domain, and a CD3ζ cytoplasmic signaling domain, and wherein the extracellular domain is a single-chain antibody fragment (scFv) that binds CD70.

46. The method of claim 45, wherein the scFv comprises a heavy chain variable domain (V_{H}) comprising SEQ ID NO: 49, and a light chain variable domain (V_{L}) comprising SEQ ID NO: 50.

47. The method of claim 46, wherein the scFv comprises SEQ ID NO: 48.

48. The method of claim 45, wherein the CAR comprises SEQ ID NO: 46.

49. The method of claim 1, wherein the disrupted TRAC gene is produced by a CRISPR/Cas9 gene editing system, which comprises a guide RNA comprising a spacer sequence of SEQ ID NO: 8 or 9.

50. The method of claim 49, wherein the disrupted TRAC gene has a deletion of the region targeted the spacer sequence of SEQ ID NO: 8 or 9, or a portion thereof.

51. The method of claim 1, wherein the disrupted β2M gene is produced by a CRISPR/Cas9 gene editing system, which comprises a guide RNA comprising a spacer sequence of SEQ ID NO: 12 or 13.

52. The method of claim 1, wherein the disrupted CD70 gene is produced by a CRISPR/Cas9 gene editing system, which comprises a guide RNA comprising a spacer sequence of SEQ ID NO: 4 or 5.


SEQUENCE LISTING (69 sequences)

SEQ ID NO: 1 - protein, 1368 aa, Streptococcus pyogenes [sequence omitted]

SEQ ID NO: 2 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuuuggucccauuggucgcguuuuagagcuagaaauagcaaguuaaaauaaggcuaguccguuaucaacuugaaaaaguggcaccgagucggugcuuuu

SEQ ID NO: 3 - RNA, 100 nt, Artificial Sequence; identical to SEQ ID NO: 2
  Synthetic

SEQ ID NO: 4 - RNA, 20 nt, Artificial Sequence
  Synthetic
  misc_feature (1)..(4): modified with 2′-O-methyl phosphorothioate
gcuuuggucccauuggucgc

SEQ ID NO: 5 - RNA, 20 nt, Artificial Sequence
  Synthetic
gcuuuggucccauuggucgc

SEQ ID NO: 6 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
agagcaacagugcuguggccguuuuagagcuagaaauagcaaguuaaaauaaggcuaguccguuaucaacuugaaaaaguggcaccgagucggugcuuuu

SEQ ID NO: 7 - RNA, 100 nt, Artificial Sequence; identical to SEQ ID NO: 6
  Synthetic

SEQ ID NO: 8 - RNA, 20 nt, Artificial Sequence
  Synthetic
  misc_feature (1)..(4): modified with 2′-O-methyl phosphorothioate
agagcaacagugcuguggcc

SEQ ID NO: 9 - RNA, 20 nt, Artificial Sequence
  Synthetic
agagcaacagugcuguggcc

SEQ ID NO: 10 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuacucucucuuucuggccguuuuagagcuagaaauagcaaguuaaaauaaggcuaguccguuaucaacuugaaaaaguggcaccgagucggugcuuuu

SEQ ID NO: 11 - RNA, 100 nt, Artificial Sequence; identical to SEQ ID NO: 10
  Synthetic

SEQ ID NO: 12 - RNA, 20 nt, Artificial Sequence
  Synthetic
  misc_feature (1)..(4): modified with 2′-O-methyl phosphorothioate
gcuacucucucuuucuggcc

SEQ ID NO: 13 - RNA, 20 nt, Artificial Sequence
  Synthetic
gcuacucucucuuucuggcc

SEQ ID NO: 14 - DNA, 23 bp, Artificial Sequence
  Synthetic
gctttggtcccattggtcgcggg

SEQ ID NO: 15 - DNA, 20 bp, Artificial Sequence
  Synthetic
gctttggtcccattggtcgc

SEQ ID NO: 16 - DNA, 23 bp, Artificial Sequence
  Synthetic
agagcaacagtgctgtggcctgg

SEQ ID NO: 17 - DNA, 20 bp, Artificial Sequence
  Synthetic
agagcaacagtgctgtggcc

SEQ ID NO: 18 - DNA, 23 bp, Artificial Sequence
  Synthetic
gctactctctctttctggcctgg

SEQ ID NO: 19 - DNA, 20 bp, Artificial Sequence
  Synthetic
gctactctctctttctggcc

SEQ ID NO: 20 - RNA, 100 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 21 - RNA, 96 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 22 - RNA, 114 nt, Artificial Sequence
  Synthetic
  misc_feature (1)..(17): n is a, c, g, or u
  misc_feature (18)..(30): may be absent
  misc_feature (109)..(114): may be absent
nnnnnnnnnnnnnnnnnnnnnnnnnnnnnnguuuuagagcuagaaauagcaaguuaaaauaaggcuaguccguuaucaacuugaaaaaguggcaccgagucggugcuuuuuuuu

SEQ ID NO: 23 - DNA, 19 bp, Artificial Sequence
  Synthetic
aagagcaacaaatctgact

SEQ ID NO: 24 - DNA, 39 bp, Artificial Sequence
  Synthetic
aagagcaacagtgctgtgcctggagcaacaaatctgact

SEQ ID NO: 25 - DNA, 33 bp, Artificial Sequence
  Synthetic
aagagcaacagtgctggagcaacaaatctgact

SEQ ID NO: 26 - DNA, 34 bp, Artificial Sequence
  Synthetic
aagagcaacagtgcctggagcaacaaatctgact

SEQ ID NO: 27 - DNA, 19 bp, Artificial Sequence
  Synthetic
aagagcaacagtgctgact

SEQ ID NO: 28 - DNA, 41 bp, Artificial Sequence
  Synthetic
aagagcaacagtgctgtgggcctggagcaacaaatctgact

SEQ ID NO: 29 - DNA, 38 bp, Artificial Sequence
  Synthetic
aagagcaacagtgctggcctggagcaacaaatctgact

SEQ ID NO: 30 - DNA, 41 bp, Artificial Sequence
  Synthetic
aagagcaacagtgctgtgtgcctggagcaacaaatctgact

SEQ ID NO: 31 - DNA, 79 bp, Artificial Sequence
  Synthetic
cgtggccttagctgtgctcgcgctactctctctttctgcctggaggctatccagcgtgagtctctcctaccctcccgct

SEQ ID NO: 32 - DNA, 78 bp, Artificial Sequence
  Synthetic
cgtggccttagctgtgctcgcgctactctctctttcgcctggaggctatccagcgtgagtctctcctaccctcccgct

SEQ ID NO: 33 - DNA, 75 bp, Artificial Sequence
  Synthetic
cgtggccttagctgtgctcgcgctactctctctttctggaggctatccagcgtgagtctctcctaccctcccgct

SEQ ID NO: 34 - DNA, 84 bp, Artificial Sequence
  Synthetic
cgtggccttagctgtgctcgcgctactctctctttctggatagcctggaggctatccagcgtgagtctctcctaccctcccgct

SEQ ID NO: 35 - DNA, 55 bp, Artificial Sequence
  Synthetic
cgtggccttagctgtgctcgcgctatccagcgtgagtctctcctaccctcccgct

SEQ ID NO: 36 - DNA, 82 bp, Artificial Sequence
  Synthetic
cgtggccttagctgtgctcgcgctactctctctttctgtggcctggaggctatccagcgtgagtctctcctaccctcccgct

SEQ ID NO: 37 - DNA, 58 bp, Artificial Sequence
  Synthetic
cacaccacgaggcagatcaccaagcccgcgcaatgggaccaaagcagcccgcaggacg

SEQ ID NO: 38 - DNA, 61 bp, Artificial Sequence
  Synthetic
cacaccacgaggcagatcaccaagcccgcgaaccaatgggaccaaagcagcccgcaggacg

SEQ ID NO: 39 - DNA, 48 bp, Artificial Sequence
  Synthetic
cacaccacgaggcagatcaccaatgggaccaaagcagcccgcaggacg

SEQ ID NO: 40 - DNA, 59 bp, Artificial Sequence
  Synthetic
cacaccacgaggcagatcaccaagcccgcgccaatgggaccaaagcagcccgcaggacg

SEQ ID NO: 41 - DNA, 59 bp, Artificial Sequence
  Synthetic
cacaccacgaggcagatcaccaagcccgcaccaatgggaccaaagcagcccgcaggacg

SEQ ID NO: 42 - DNA, 35 bp, Artificial Sequence
  Synthetic
cacaccacgaggcagatcaccaagcccgcaggacg

SEQ ID NO: 43 - DNA, 4688 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - DNA, 4364 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 45 - DNA, 1527 bp, Artificial Sequence
  Synthetic
atggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataa

SEQ ID NO: 46 - protein, 508 aa, Artificial Sequence
  Synthetic
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 735 bp, Artificial Sequence
  Synthetic
caggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaa

SEQ ID NO: 48 - protein, 245 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 49 - protein, 118 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 50 - protein, 111 aa, Artificial Sequence
  Synthetic
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 51 - DNA, 16 bp, Artificial Sequence
  Synthetic
ggggsggggsggggsg

SEQ ID NO: 52 - protein, 22 aa, Artificial Sequence
  Synthetic
MLLLVTSLLLCELPHPAFLLIP

SEQ ID NO: 53 - protein, 21 aa, Artificial Sequence
  Synthetic
MALPVTALLLPLALLLHAARP

SEQ ID NO: 54 - protein, 84 aa, Artificial Sequence
  Synthetic
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 55 - protein, 23 aa, Artificial Sequence
  Synthetic
IYIWAPLAGTCGVLLLSLVITLY

SEQ ID NO: 56 - DNA, 126 bp, Artificial Sequence
  Synthetic
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 57 - protein, 42 aa, Artificial Sequence
  Synthetic
KRGRKKLLYIFKQPFMRPVQTTQEEDGCSCRFPEEEEGGCEL

SEQ ID NO: 58 - DNA, 120 bp, Artificial Sequence
  Synthetic
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 59 - protein, 40 aa, Artificial Sequence
  Synthetic
SKRSRLLHSDYMNMTPRRPGPTRKHYQPYAPPRDFAAYRS

SEQ ID NO: 60 - DNA, 336 bp, Artificial Sequence
  Synthetic
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 61 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 800 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 63 - DNA, 1178 bp, Artificial Sequence
  Synthetic
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 64 - DNA, 49 bp, Artificial Sequence
  Synthetic
aataaaatcgctatccatcgaagatggatgtgtgttggttttttgtgtg

SEQ ID NO: 65 - DNA, 804 bp, Artificial Sequence
  Synthetic
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 66 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcccgcaggacgcacccauaguuuuagagcuagaaauagcaaguuaaaauaaggcuaguccguuaucaacuugaaaaaguggcaccgagucggugcuuuu

SEQ ID NO: 67 - RNA, 100 nt, Artificial Sequence; identical to SEQ ID NO: 66
  Synthetic

SEQ ID NO: 68 - RNA, 20 nt, Artificial Sequence
  Synthetic
  misc_feature (1)..(4): modified with 2′-O-methyl phosphorothioate
gcccgcaggacgcacccaua

SEQ ID NO: 69 - RNA, 20 nt, Artificial Sequence
  Synthetic
gcccgcaggacgcacccaua